(19) United States
(12) Patent Application Publication
(10) Pub. No.: US 2023/0287510 A1
(43) Pub. Date: Sep. 14, 2023

(54) COMPOSITIONS, PANELS, AND METHODS FOR CHARACTERIZING CHRONIC LYMPHOCYTIC LEUKEMIA

(71) Applicants: Catherine J. WU, Boston, MA (US); Gad GETZ, Boston, MA (US); Binyamin A. KNISBACHER, Cambridge, MA (US); Ziao LIN, Cambridge, MA (US); Cynthia K. HAHN, Boston, MA (US); The Broad Institute, Inc., Cambridge, MA (US)

(72) Inventors: Catherine J. WU, Boston, MA (US); Gad GETZ, Boston, MA (US); Binyamin A. KNISBACHER, Cambridge, MA (US); Ziao LIN, Cambridge, MA (US); Cynthia K. HAHN, Boston, MA (US)

(73) Assignees: The Broad Institute, Inc., Cambridge, MA (US); Dana-Farber Cancer Institute, Inc., Boston, MA (US); The General Hospital Corporation, Boston, MA (US); President and Fellows of Harvard College, Cambridge, MA (US)

(21) Appl. No.: 18/020,587

(22) PCT Filed: Aug. 9, 2021
(86) PCT No.: PCT/US2021/045144

§ 371 Date: Feb. 9, 2023

Related U.S. Application Data
(60) Provisional application No. 63/063,798, filed on Aug. 10, 2020.

(51) Int. Cl. C12Q 1/6886; A61K 45/06; C12Q 1/6874

(52) U.S. Cl. CPC C12Q 1/6886; A61K 45/06; C12Q 1/6874; C12Q 2600/158; C12Q 2600/154

(57) ABSTRACT

As described below, the present invention features compositions, panels of biomarkers, and methods for characterizing chronic lymphocytic leukemia (CLL) for prognosis and selection of a subject for a treatment and/or inclusion in a clinical trial.

### CROSS-REFERENCE TO RELATED APPLICATION

This application claims the benefit of the following U.S. Provisional Application No. 63/063,798, filed Aug. 10, 2020, the entire contents of which are incorporated herein by reference.

### STATEMENT OF RIGHTS TO INVENTIONS MADE UNDER FEDERALLY SPONSORED RESEARCH

This invention was made with government support under grant nos. CA206978 and HL116324 awarded by the National Institutes of Health. The government has certain rights in the invention.

### BACKGROUND OF THE INVENTION

Chronic lymphocytic leukemia (CLL) affected about 904,000 people globally in 2015 and resulted in 60,700 deaths. CLL is a B cell neoplasm with variable natural history that is conventionally categorized into two major subtypes distinguished by the extent of somatic mutations in the heavy chain variable region of immunoglobulin genes. Only fragments of the “CLL map” have been studied. This lack of understanding of the disease has led to deficiencies in understanding of the disease, prognostication, and treatment assignment.

Thus, there remains a need for improved compositions and methods for characterizing chronic lymphocytic leukemia (CLL) for prognosis and selection of a subject for a specified treatment.

### SUMMARY OF THE INVENTION

As described below, the present invention features compositions, panels of biomarkers, and methods for characterizing chronic lymphocytic leukemia (CLL) for prognosis and selection of a subject for a treatment and/or inclusion in a clinical trial.

In one aspect, the invention features a panel for characterizing chronic lymphocytic leukemia in a biological sample of a subject. The panel contains two or more polypeptide markers selected from one or more of: ABCA9, ACAP3, ACSM3, ADAP2, AF127936.7, ARHGAP33, ARMC7, ARRDC5, ARSD, ARSI, ASB2, ATP1A3, ATP2B1, ATPIF1, BASP1, BCL2A1, BCL7A, BCS1L, CAMK2A, CLDN23, CMTM7, COBLL1, CRELD2, CRY1, CTAGE9, CTLA4, DDR1, DKFZP761J1410, DPF3, EML6, ERRFI1, ESPNL, EZH2, FAHD2B, FAM109A, FBXO27, FGL2, FLJ20373, FMOD, GADD45A, GNAO1, GPR160, GPR34, GUCD1, HCK, HDAC4, HIP1R, HMCES, IGSF3, IQSEC1, ITGAX, KCNH3, KCNN3, KCTD3, KDM1B, KLK1, KSR1, LCN10, LINC00865, LPL, LRRK2, LUZP1, MAP4K4, MAPK4, MAST4, MPRIP, MRO, MSI2, MVB12B, MYBL1, MYC, MYL5, MYL9, MYO3A, NEDD9, NFKBIZ, NR2F6, NRIP1, NRSN2, NUGGC, P2RX1, PELI3, PIGB, PIP5K1B, PITPNC1, PLD1, PTPN7, QDPR, REPS2, RHBDF2, RIMKLB, RP11-134N1.2, RP11-265P11.1, RP11-453F18_B.1, RP11-456H18.2, RP1-90J20.12, SAMSN1, SCPEP1, SH3D21, SLC44A1, SLC4A7, SLC4A8, SMIM10, SPN, SSBP3, STAM, STX5, SYNGR3, TAS1R3, TBC1D2B, TBC1D9, TFEC, TIMELESS, TNFRSF13B, TNR, TOX2, TRIM7, TUBG2, VSIG10, WNT5A, ZMYND8, and ZNF804A, fragments thereof, or polynucleotides encoding such polypeptides or fragments thereof.

In another aspect, the invention features a panel for characterizing chronic lymphocytic leukemia in a biological sample of a subject. The panel contains two or more polypeptide markers selected from one or more of: ACAP3, ACSM3, AEBP1, AKT3, ARHGAP33, ARHGAP42, ARMC7, ARRDC5, ATPIF1, BACH2, BASP1, BCL7A, C17orf100, CBLB, CD72, CD86, CEACAM1, CHPT1, CLDN7, CMTM7, CNTNAP1, COBLL1, COL18A1, CRY1, CTLA4, EGR3, EML6, EZH2, FADS3, FCER1G, FCRL2, FGL2, FLJ20373, FMOD, GADD45A, GLIPR1, GNB4, GPR160, GPR34, GRIK3, GUCD1, HCK, HIP1R, HIVEP3, HMCES, IGF2BP3, IGSF3, IL21R, INPP5F, IQGAP2, IQSEC1, ITGAX, ITGB5, JDP2, KANK2, KCNH2, KDM1B, KLF3, LATS2, LCN10, LEF1, LPL, LRRK2, LUZP1, MAP4K4, MID1IP1, MMP14, MPRIP, MSI2, MYBL1, MYL9, MYLIP, MZB1, NBPF3, NRIP1, NRSN2, NUGGC, NXPH4, P2RX1, P2RX5, P2RY14, PDGFD, PIP5K1B, PITPNC1, PON2, PRICKLE1, PTPN7, RCN3, RDX, RHBDF2, RIMKLB, RNF135, RP11-145M9.4, RP11-268J15.5, RP11-463012.3, RP5-1028K7.2, SAMSN1, SCCPDH, SCD, SCPEP1, SDC3, SECTM1, SESN3, SH3BP2, SH3D21, SLC16A5, SLC19A1, SLC4A7, SPN, SSBP3, STX5, SUSD1, TBC1D2B, TBC1D9, TBKBP1, TCF7, TFEC, TGFBR3, TIGIT, TIMELESS, TMEM133, TNFRSF13B, TOX2, TRAK2, TTC39C, TUBG2, VPS37B, VSIG10, WNT9A, ZAP70, ZNF667-AS1, ZNF804A, and ZSWIM6, fragments thereof, or polynucleotides encoding such polypeptides or fragments thereof.

In another aspect, the invention features a panel for characterizing chronic lymphocytic leukemia in a biological sample of a subject. The panel contains a set of polypeptide markers or fragments thereof, or polynucleotides encoding such polypeptides or fragments thereof, where the set of polypeptide markers is selected from one or more of the following sets: (A) an Ec-i set containing polypeptide markers GRIK3, IQGAP2, FCER1G, STK32B, GADD45A, ITGAX, KLF3, RFTN1, PTK2, DFNB31, and ZMAT1; (B) an EC-m1 set containing polypeptide markers TFEC, COL18A1, SLC19A1, NRIP1, KCNH2, P2RX1, ARRDC5, BEX4, and APP; (C) an Ec-m2 set containing polypeptide markers EML6, HCK, CD1C, VPS37B, CYBB, NXPH4, BTNL9, KLRK1, IQSEC1, BANK1, LEF1, SH3D21, FMOD, SEMA4A, CTLA4, ADTRP, IGSF3, IGFBP4, PDGFD, and APOD; (D) an Ec-m3 set containing polypeptide markers MS4A4E, MYL9, NT5E, MS4A6A, PITPNC1, CNTNAP2, IGF2BP3, WNT3, CLDN7, TCF7, BASP1, FLJ20373, MAP4K4, LRRK2, SAMSN1, CEACAM1, TNFRSF13B, PHF16, MID1IP1, and ABCA9; (E) an Ec-m4 set containing polypeptide markers MYBL1, NUGGC, GNG8, AEBP1, HIP1R, LATS2, RIMKLB, EML6, FADS3, MBOAT1, LCN10, DCLK2, and GLUL; (F) an Ec-o set containing of polypeptide markers ACSM3, TOX2, PHF16, SESN3, TBC1D9, PIP5K1B, SIK1, DUSP5, GNG7, HIVEP3, MARCKSL1, GPR183, HRK, and PITPNC1; (G) an Ec-u1 set containing polypeptide markers SEPT10, LDOC1, LPL, KANK2, SOWAHC, DUSP26, OSBPL5, WNT9A, FGFR1, GTSF1L, ADD3, AKT3, COBLL1, MNDA, FCRL3, FAM49A, FCRL2, SLC2A3, and MARCKS; and (H) an Ec-u2 set containing polypeptide markers ITGB5, BCL7A, PPP1R9A, TSPAN13, SLC12A7, SSBP3, VASH1, SPG20, IL13RA1, NR3C2, TUBG2, ZNF804A, and IL2RA.

In another aspect, the invention features a method of characterizing a chronic lymphocytic leukemia (CLL). The method involves actions (A) and (B). Action (A) involves measuring the level of each of a set of markers in a biological sample, where the set of biomarkers contains two or more of markers selected from one or more of ABCA9, ACAP3, ACSM3, ADAP2, AF127936.7, ARHGAP33, ARMC7, ARRDC5, ARSD, ARSI, ASB2, ATP1A3, ATP2B1, ATPIF1, BASP1, BCL2A1, BCL7A, BCS1L, CAMK2A, CLDN23, CMTM7, COBLL1, CRELD2, CRY1, CTAGE9, CTLA4, DDR1, DKFZP761J1410, DPF3, EML6, ERRFI1, ESPNL, EZH2, FAHD2B, FAM109A, FBXO27, FGL2, FLJ20373, FMOD, GADD45A, GNAO1, GPR160, GPR34, GUCD1, HCK, HDAC4, HIP1R, HMCES, IGSF3, IQSEC1, ITGAX, KCNH3, KCNN3, KCTD3, KDM1B, KLK1, KSR1, LCN10, LINC00865, LPL, LRRK2, LUZP1, MAP4K4, MAPK4, MAST4, MPRIP, MRO, MSI2, MVB12B, MYBL1, MYC, MYL5, MYL9, MYO3A, NEDD9, NFKBIZ, NR2F6, NRIP1, NRSN2, NUGGC, P2RX1, PELI3, PIGB, PIP5K1B, PITPNC1, PLD1, PTPN7, QDPR, REPS2, RHBDF2, RIMKLB, RP11-134N1.2, RP11-265P11.1, RP11-453F18_B.1, RP11-456H18.2, RP1-90J20.12, SAMSN1, SCPEP1, SH3D21, SLC44A1, SLC4A7, SLC4A8, SMIM10, SPN, SSBP3, STAM, STX5, SYNGR3, TAS1R3, TBC1D2B, TBC1D9, TFEC, TIMELESS, TNFRSF13B, TNR, TOX2, TRIM7, TUBG2, VSIG10, WNT5A, ZMYND8, and ZNF804A. Action (B) involves using the measured levels to classify the CLL as having an expression subtype selected from Ec-i, EC-m1, EC-m2, EC-m3, EC-m4, EC-o, EC-u1, or EC-u2, thereby characterizing the CLL.

In another aspect, the invention features a method of characterizing a chronic lymphocytic leukemia (CLL). The method involves actions (A) and (B). Action (A) involves measuring the level of each of a set of markers in a biological sample, where the set contains two or more of markers selected from ACAP3, ACSM3, AEBP1, AKT3, ARHGAP33, ARHGAP42, ARMC7, ARRDC5, ATPIF1, BACH2, BASP1, BCL7A, C17orf100, CBLB, CD72, CD86, CEACAM1, CHPT1, CLDN7, CMTM7, CNTNAP1, COBLL1, COL18A1, CRY1, CTLA4, EGR3, EML6, EZH2, FADS3, FCER1G, FCRL2, FGL2, FLJ20373, FMOD, GADD45A, GLIPR1, GNB4, GPR160, GPR34, GRIK3, GUCD1, HCK, HIP1R, HIVEP3, HMCES, IGF2BP3, IGSF3, IL21R, INPP5F, IQGAP2, IQSEC1, ITGAX, ITGB5, JDP2, KANK2, KCNH2, KDM1B, KLF3, LATS2, LCN10, LEF1, LPL, LRRK2, LUZP1, MAP4K4, MID1IP1, MMP14, MPRIP, MSI2, MYBL1, MYL9, MYLIP, MZB1, NBPF3, NRIP1, NRSN2, NUGGC, NXPH4, P2RX1, P2RX5, P2RY14, PDGFD, PIP5K1B, PITPNC1, PON2, PRICKLE1, PTPN7, RCN3, RDX, RHBDF2, RIMKLB, RNF135, RP11-145M9.4, RP11-268J15.5, RP11-463012.3, RP5-1028K7.2, SAMSN1, SCCPDH, SCD, SCPEP1, SDC3, SECTM1, SESN3, SH3BP2, SH3D21, SLC16A5, SLC19A1, SLC4A7, SPN, SSBP3, STX5, SUSD1, TBC1D2B, TBC1D9, TBKBP1, TCF7, TFEC, TGFBR3, TIGIT, TIMELESS, TMEM133, TNFRSF13B, TOX2, TRAK2, TTC39C, TUBG2, VPS37B, VSIG10, WNT9A, ZAP70, ZNF667-AS1, ZNF804A, and ZSWIM6. Action (B) involves using the measured levels to classify the CLL as having an expression subtype selected from Ec-i, EC-m1, EC-m2, EC-m3, EC-m4, EC-o, EC-u1, or EC-u2, thereby characterizing the CLL.

In one aspect, the invention features a method of characterizing a chronic lymphocytic leukemia (CLL), the method involves actions (A) and (B). Action (A) involves measuring the level of each of a set of biomarkers in a biological sample, where the set of biomarkers contains: (i) an Ec-i set containing polypeptide markers GRIK3, IQGAP2, FCER1G, STK32B, GADD45A, ITGAX, KLF3, RFTN1, PTK2, DFNB31, and ZMAT1; (ii) an EC-m1 set containing polypeptide markers TFEC, COL18A1, SLC19A1, NRIP1, KCNH2, P2RX1, ARRDC5, BEX4, and APP; (iii) an Ec-m2 set containing polypeptide markers EML6, HCK, CD1C, VPS37B, CYBB, NXPH4, BTNL9, KLRK1, IQSEC1, BANK1, LEF1, SH3D21, FMOD, SEMA4A, CTLA4, ADTRP, IGSF3, IGFBP4, PDGFD, and APOD; (iv) an Ec-m3 set containing polypeptide markers MS4A4E, MYL9, NT5E, MS4A6A, PITPNC1, CNTNAP2, IGF2BP3, WNT3, CLDN7, TCF7, BASP1, FLJ20373, MAP4K4, LRRK2, SAMSN1, CEACAM1, TNFRSF13B, PHF16, MID1IP1, and ABCA9; (v) an Ec-m4 set containing polypeptide markers MYBL1, NUGGC, GNG8, AEBP1, HIP1R, LATS2, RIMKLB, EML6, FADS3, MBOAT1, LCN10, DCLK2, and GLUL; (vi) an Ec-o set containing polypeptide markers ACSM3, TOX2, PHF16, SESN3, TBC1D9, PIP5K1B, SIK1, DUSP5, GNG7, HIVEP3, MARCKSL1, GPR183, HRK, and PITPNC1; (vii) an Ec-u1 set containing polypeptide markers SEPT10, LDOC1, LPL, KANK2, SOWAHC, DUSP26, OSBPL5, WNT9A, FGFR1, GTSF1L, ADD3, AKT3, COBLL1, MNDA, FCRL3, FAM49A, FCRL2, SLC2A3, and MARCKS; and/or (viii) an Ec-u2 set containing polypeptide markers ITGB5, BCL7A, PPP1R9A, TSPAN13, SLC12A7, SSBP3, VASH1, SPG20, IL13RA1, NR3C2, TUBG2, ZNF804A, and IL2RA. Action (B) involves using the measured levels to classify the CLL as having an expression subtype selected from Ec-i, EC-m1, EC-m2, EC-m3, EC-m4, EC-o, EC-u1, or EC-u2, thereby characterizing the CLL.

In another aspect, the invention features a method for selecting a subject having chronic lymphocytic leukemia (CLL) for inclusion in or exclusion from a clinical trial. The method involves actions (A) and (B). Action (A) involves characterizing the CLL according to the method of any one of claims 9-32 to determine the expression subtype of the CLL. Action (B) involves selecting the subject for inclusion in the clinical trial if the CLL has an expression subtype associated with sensitivity to a drug used in the clinical trial, and excluding the subject from the clinical trial if the CLL has an expression subtype associated with resistance to a drug used in the clinical trial.

In another aspect, the invention features a method for treating a selected subject having chronic lymphocytic leukemia (CLL). The method involves administering an agent to a selected subject, where the subject is selected for treatment by characterizing marker expression in a biological sample of the subject using a panel of any of the above aspects.

In another aspect, the invention features a panel of capture molecules, where each capture molecule binds a marker of any one of the above aspects.

In another aspect, the invention features a kit for characterizing a chronic lymphocytic leukemia (CLL). The kit contains a set of capture molecules each of which specifically binds biomarkers of the panel of any of the above aspects.

In any of the above aspects, the markers are bound to a capture molecule. In embodiments, the capture molecule is bound to a substrate. In embodiments, the capture molecules contain an antibody or antigen binding fragment thereof. In embodiments, the capture molecules contain a polynucleotide.

In any of the above aspects, action (B) further involves using the level of each biomarker as an input to a classifier to determine the expression subtype. In embodiments, the classifier is a machine learning classifier.

In any of the above aspects, the biological sample contains a liquid sample or a tissue sample. In any of the above aspects, the biological sample contains a blood, blood serum, or plasma sample. In any of the above aspects, the biological sample contains a homogenized tissue sample. In embodiments, the tissue sample is derived from a biopsy sample.

In any of the above aspects, the levels are measured relative to a reference sample. In embodiments, the reference sample is a corresponding biological sample derived from a healthy subject.

In any of the above aspects, the levels are measured using polynucleotide sequencing. In embodiments, the polynucleotide sequencing is RNA-seq. In embodiments, the polynucleotide sequencing is targeted sequencing. In any of the above aspects, the levels are measured using an immunoassay or affinity capture. In any of the above aspects, the levels are measured using a biochip. In embodiments, the biochip is a protein biochip or a nucleic acid biochip. In any of the above aspects, the levels are measured using mass spectroscopy. In any of the above aspects, the levels are measured using a capture molecule. In embodiments, the capture molecule contains a molecular identifier. In embodiments, the molecular identifier contains a fluorescent molecule. In any of the above aspects, the method involves detecting the molecular identifier using FACS. In any of the above aspects, the method involves measuring the levels using a NanoString assay. In any of the above aspects, measuring the levels is carried out on a plate, chip, beads, microfluidic platform, membrane, planar microarray, or suspension array.

In any of the above aspects, the agent is a kinase inhibitor. In any of the above aspects, the agent is a B-cell receptor pathway inhibitor. In any of the above aspects, the agent targets a DNA damage response, PI3K/AKT, cell cycle control, apoptosis, BCR/ABL, HSP90, or MAPK.

In any of the above aspects, the drug sensitivity or drug resistance of the chronic lymphocytic leukemia (CLL) is determined according to Tables 7A and/or 7B.

In any of the above aspects, the agent is selected from one or more of 1-Ter-Butyl-3-P-Tolyl-1h-Pyrazolo[3,4-D]Pyrimidin-4-Ylamine, 4-HYDROXY-N′-(4-ISOPROPYLBENZYL)BENZOHYDRAZIDE, actinomycin D, afatinib, Amsacrine, and/or Vernakalant, Astemizole, AT13387, AZD7762, Azimilide, BAY 11-7085, Bepridil, Betrixaban, Bosutinib, BX912, Carvedilol, CCT241533, cephaeline, chaetoglobosin A, Chlorobutanol, Chlorpromazine, Ciprofloxacin, Cisapride, Clarithromycin, Cytarabine, dasatinib, Disopyramide, Dofetilide, Doxepin, Dronedarone, duvelisib, Erythromycin, everolimus, Flecainide, fludarabine, Fluoxetine, Fluvoxamine, Fostamatinib, Halofantrine, Hydroxyzine, ibrutinib, Ibutilide, idelalisib, Imipramine, Isavuconazole, Ketoconazole, KU-60019, KX2-391, Levomefolic acid, Loratadine, Methotrexate, MIS-43, MK-1775, MK-2206, navitoclax, Nefazodone, Nitazoxanide, NU7441, Pentoxifylline, Pentoxyverine, Perhexiline, PF 477736, Phenytoin, Phosphonotyrosine, Pimozide, Pitolisant, Potassium nitrate, Pralatrexate, Prazosin, Procainamide, Propafenone, PRT062607 HCl, Quercetin, Quinidine, rotenone, saracatinib, SD07, See comments, selumetinib, Semaglutide, Sertindole, SGI-1776, SNS-032, Sotalol, spebrutinib, TAE684, tamatinib, Tamoxifen, Tecastemizole, Terazosin, Terfenadine, thapsigargin, Thioridazine, Topiramate, Trimetrexate, venetoclax, Verapamil, vorinostat, and YM155. In any of the above aspects, the agent is selected from one or more of AT13387, AZD7762, dasatinib, duvelisib, fludarabine, ibrutinib, idelalisib, navitoclax, PRT062607 HCl, selumetinib, SNS-032, or venetoclax.

In any of the above aspects, the agent used in the clinical trial is fludarabine, and, if the lymphocytic leukemia (CLL) has the expression subtype EC-m3, the subject is selected for inclusion in the clinical trial. In any of the above aspects, the drug used in the clinical trial targets the B cell receptor pathway or PI3K/AKT, and, if the lymphocytic leukemia (CLL) has the expression subtype EC-m3, the subject is excluded from the clinical trial. In any of the above aspects, the drug used in the clinical trial is ibrutinib or idelalisib, and, if the lymphocytic leukemia (CLL) has the expression subtype EC-m3, the subject is excluded from the clinical trial. In any of the above aspects, the drug used in the clinical trial targets CDK2/7/9, and, if the lymphocytic leukemia (CLL) has the expression subtype EC-m4, the subject selected for inclusion in the clinical trial. In any of the above aspects, the drug used in the clinical trial is SNS-032, and, if the lymphocytic leukemia (CLL) has the expression subtype EC-m4, the subject selected for inclusion in the clinical trial. In any of the above aspects, the drug used in the clinical trial targets the B cell receptor pathway or BTK, and, if the lymphocytic leukemia (CLL) has the expression subtype EC-m4, the subject is excluded from the clinical trial. In any of the above aspects, the drug used in the clinical trial is ibrutinib, and, if the lymphocytic leukemia (CLL) has the expression subtype EC-m4, the subject is excluded from the clinical trial. In any of the above aspects, the drug used in the clinical trial targets apoptosis, BH3, and/or survivin, and, if the lymphocytic leukemia (CLL) has the expression subtype EC-u1, the subject is excluded from the clinical trial. In any of the above aspects, the drug used in the clinical trial is venetoclax or navitoclax, and if the lymphocytic leukemia (CLL) has the expression subtype EC-u1, the subject is excluded from the clinical trial. In any of the above aspects, the drug used in the clinical trial targets DNA damage response, the B-cell receptor pathway, MAPK, PI3K/AKT, HSP90, or BCR/ABL, and if the lymphocytic leukemia (CLL) has the expression subtype EC-u2, the subject is selected for inclusion in the clinical trial. In any of the above aspects, the drug used in the clinical trial is AZD7762, dasatinib, AT13387, ibrutinib, duvelisib, idelalisib, selumetinib, or PRT062607 HCl, and if the lymphocytic leukemia (CLL) has the expression subtype EC-u2, the subject is selected for inclusion in the clinical trial.

In any of the above aspects, the subject is selected for administration of fludarabine if the expression subtype is EC-m3. In any of the above aspects, the subject is selected for administration of a drug targeting CDK2/7/9 if the expression subtype is EC-m4. In any of the above aspects, the subject is selected for administration of SNS-032 if the expression subtype is EC-m4. In any of the above aspects, the subject is selected for administration of a drug targeting DNA damage response, the B-cell receptor pathway, MAPK, PI3K/AKT, HSP90, or BCR/ABL if the expression subtype is EC-u2. In any of the above aspects, the subject is selected for administration of AZD7762, dasatinib, AT13387, ibrutinib, duvelisib, idelalisib, selumetinib, or PRT062607 HCl if the expression subtype is EC-u2.

In any of the above aspects, if the CLL has an expression subtype associated with NRIP1, the subject is selected for administration of 4-HYDROXY-N′-(4-ISOPROPYLBENZYL)BENZOHYDRAZIDE. In any of the above aspects, if the CLL has an expression subtype associated with SLC19A1, the subject is selected for administration of an agent selected from one or more of Pralatrexate, Methotrexate, Levomefolic acid, Nitazoxanide, and Trimetrexate. In any of the above aspects, if the CLL has an expression subtype associated with KCNH2, the subject is selected for administration of an agent selected from one or more of Amsacrine, Astemizole, Azimilide, Bepridil, Betrixaban, Carvedilol, Chlorobutanol, Chlorpromazine, Ciprofloxacin, Cisapride, Clarithromycin, Disopyramide, Dofetilide, Doxepin, Dronedarone, Erythromycin, Flecainide, Fluoxetine, Fluvoxamine, Halofantrine, Hydroxyzine, Ibutilide, Imipramine, Isavuconazole, Ketoconazole, Loratadine, Nefazodone, Pentoxyverine, Perhexiline, Phenytoin, Pimozide, Pitolisant, Potassium nitrate, Prazosin, Procainamide, Propafenone, Quinidine, Sertindole, Sotalol, Tamoxifen, Tecastemizole, Terazosin, Terfenadine, Thioridazine, Verapamil, and Vernakalant. In any of the above aspects, if the CLL has an expression subtype associated with LPL, the subject is selected for administration of Semaglutide. In any of the above aspects, if the CLL has an expression subtype associated with HCK, the subject is selected for administration of an agent selected from one or more of 1-Ter-Butyl-3-P-Tolyl-1h-Pyrazolo[3,4-D]Pyrimidin-4-Ylamine, Phosphonotyrosine, Quercetin, Bosutinib, and Fostamatinib. In any of the above aspects, if the CLL has an expression subtype associated with NT5E, the subject is selected for administration of an agent selected from one or more of Pentoxifylline, and Cytarabine. In any of the above aspects, if the CLL has an expression subtype associated with GRIK3, the subject is selected for administration of Topiramate.

The invention provides compositions, panels of biomarkers, and methods for characterizing chronic lymphocytic leukemia (CLL) for prognosis and selection of a subject for a treatment and/or inclusion in a clinical trial. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The terms “biomarker” and “marker” are used interchangeably herein to refer to a protein, nucleic acid molecule, clinical indicator, or other analyte that is associated with a disease. In one embodiment, a marker of chronic lymphocytic leukemia (CLL) is differentially present in a biological sample obtained from a subject having or at risk of developing chronic lymphocytic leukemia (CLL) relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. Biomarkers can be used to classify a chronic lymphocytic leukemia (CLL). The differential presence of a marker of the invention in a subject sample can be useful in characterizing the subject as having or at risk of developing chronic lymphocytic leukemia (CLL), for determining the prognosis of the subject, for evaluating therapeutic efficacy, or for selecting a treatment regimen (e.g., selecting that the subject be evaluated and/or treated by a surgeon that specializes in chronic lymphocytic leukemia (CLL)). The invention includes markers that share at least about 85%, 90%, 95% or even 99% to a polypeptide sequence corresponding to a biomarker listed in any of Tables 3A-3B and 4. The invention includes markers that share at least about 85%, 90%, 95% or even 99% to a polynucleotide sequence corresponding to a gene listed in any of Tables 3A-3B and 4.

By “AT13387” is meant a chemical corresponding to CAS No. 912999-49-6, having the chemical structure

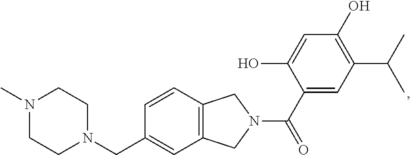 [SMILES: CC(C)c1cc(C(=O)N2Cc3ccc(CN4CCN(C)CC4)cc3C2)c(O)cc1O]

and pharmaceutically acceptable salts thereof.

By “AZD7762” is meant a chemical corresponding to CAS No. 860352-01-8, having the chemical structure

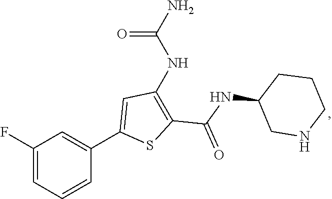 [SMILES: NC(=O)Nc1cc(-c2cccc(F)c2)sc1C(=O)N[C@H]1CCCNC1]

and pharmaceutically acceptable salts thereof.

By “dasatinib” is meant a chemical corresponding to CAS No. 302962-49-8, having the chemical structure

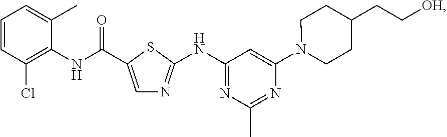 [SMILES: Cc1nc(Nc2ncc(C(=O)Nc3c(C)cccc3Cl)s2)cc(N2CCC(CCO)CC2)n1]

and pharmaceutically acceptable salts thereof.

By “duvelisib” is meant a chemical corresponding to CAS No. 1201438-56-3, having the chemical structure

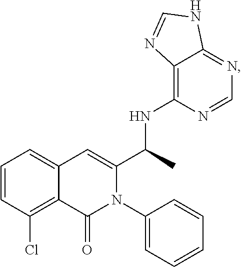 [SMILES: C[C@H](Nc1ncnc2[nH]cnc12)c1cc2cccc(Cl)c2c(=O)n1-c1ccccc1]

and pharmaceutically acceptable salts thereof.

By “fludarabine” is meant a chemical corresponding to CAS No. 21679-14-1, having the chemical structure

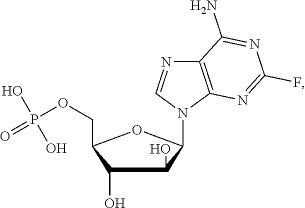

and pharmaceutically acceptable salts thereof.

By “ibrutinib” is meant a chemical corresponding to CAS No. 936563-96-1, having the chemical structure

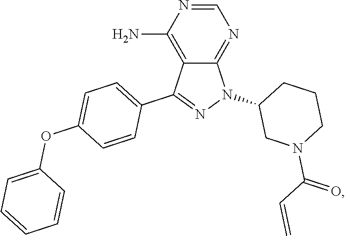 [SMILES: C=CC(=O)N1CCC[C@@H](n2nc(-c3ccc(Oc4ccccc4)cc3)c3c(N)ncnc32)C1]

and pharmaceutically acceptable salts thereof.

By “idelalisib” is meant a chemical corresponding to CAS No. 870281-82-6, having the chemical structure

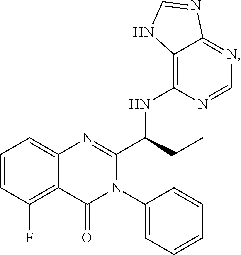 [SMILES: CC[C@H](Nc1ncnc2nc[nH]c12)c1nc2cccc(F)c2c(=O)n1-c1ccccc1]

and pharmaceutically acceptable salts thereof.

By “navitoclax” is meant a chemical corresponding to CAS No. 923564-51-6, having the chemical structure

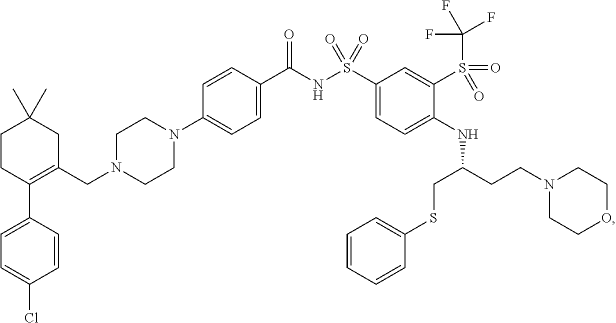 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

and pharmaceutically acceptable salts thereof.

By “PRT062607 HCL” is meant a chemical corresponding to CAS No. 1370261-97-4, having the chemical structure

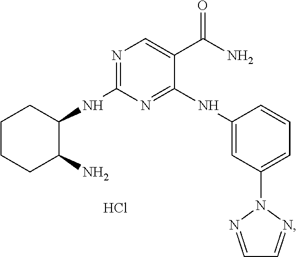 [SMILES: NC(=O)c1cnc(N[C@@H]2CCCC[C@@H]2N)nc1Nc1cccc(-n2nccn2)c1 ; Cl]

and pharmaceutically acceptable salts thereof.

By “selumetinib” is meant a chemical corresponding to CAS No. 606143-52-6, having the chemical structure

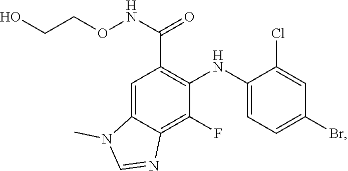 [SMILES: Cn1cnc2c(F)c(Nc3ccc(Br)cc3Cl)c(C(=O)NOCCO)cc21]

and pharmaceutically acceptable salts thereof.

By “SNS-032” is meant a chemical corresponding to CAS No. 345627-80-7, having the chemical structure

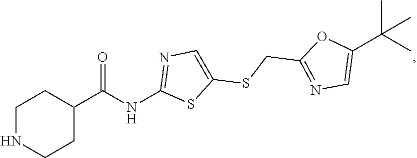 [SMILES: CC(C)(C)c1cnc(CSc2cnc(NC(=O)C3CCNCC3)s2)o1]

and pharmaceutically acceptable salts thereof.

By “venetoclax” is meant a chemical corresponding to CAS No. 1257044-40-8, having the chemical structure

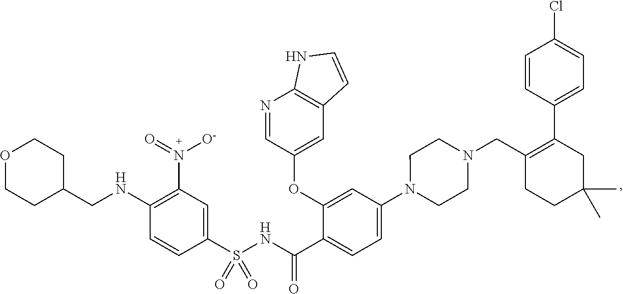

and pharmaceutically acceptable salts thereof.

By “agent” is meant any small molecule chemical compound, antibody, nucleic acid molecule, or polypeptide, or fragments thereof.

By “ameliorate” is meant to decrease, suppress, attenuate, diminish, arrest, or stabilize the development or progression of a disease.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “biological sample” is meant any tissue, cell, fluid, or other material derived from an organism. Non-limiting examples of biological samples include a bodily fluid (such as blood, blood serum, plasma, saliva, urine, ascites, cyst fluid, and the like); a homogenized tissue sample (e.g., a tissue sample obtained by biopsy); and a cell isolated from a patient sample.

By “capture molecule” or “capture reagent” is meant a reagent that specifically binds a nucleic acid molecule or polypeptide to label, select, or isolate the nucleic acid molecule or polypeptide. Non-limiting examples of capture molecules include polynucleotide probes, antibodies, and fragments thereof.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of the analyte to be detected.

By “molecular identifier” is meant an agent that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include chronic lymphocytic leukemia and the like.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increase” is meant to alter positively An increase may be by about or at least about 0.5%, 1%, 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker profile” is meant a characterization of the expression or expression level of two or more polypeptides or polynucleotides in a sample.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

“Primer set” means a set of oligonucleotides. A primer set may comprise at least about 2, 4, 6, 8, 10, 12, 14, 16, 18, 20, 30, 40, 50, 60, 80, 100, 200, 250, 300, 400, 500, 600, or more primers. In embodiments, the primers are used for detection of a biomarker(s) in a sample (e.g., by PCR, targeted sequencing, biochip, or any of various other methods described herein or combinations thereof).

By “reduce” is meant to alter negatively A reduction may be by about or at least about 0.5%, 1%, 5%, 10%, 25%, 30%, 50%, 75%, or even by 100%.

By “reference” is meant a standard or control condition. In embodiments, the reference is the level of an analyze present in a sample obtained from a subject prior to being administered a treatment, obtained from a healthy subject (e.g., a subject not having a chronic lymphocytic leukemia (CLL)), or a sample obtained from a subject at an earlier time point than a particular sample time point.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant an agent that recognizes and binds a polypeptide or polynucleotide of the invention, but which does not substantially recognize and bind other molecules in a sample, for example, a biological sample, which naturally includes a polypeptide or polynucleotide described herein.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, 80%, 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E provide plots, a genomic landscape diagram, and protein structures demonstrating that increased power enables chronic lymphocytic leukemia (CLL) driver gene detection. FIGS. 1A and 1B provide plots demonstrating that by down-sampling analysis, driver gene (FIG. 1A) and somatic copy number alteration (sCNA) (FIG. 1B) discovery increased with additional samples. Points represent a random subset of samples with smoothed fit line; analysis separated by frequency. FIG. 1C provides a genomic landscape diagram showing the landscape of genetic alterations in CLL with frequency of alterations (right, n=1064 patients). Header tracks—annotation of cohort, IGHV (heavy chain variable region of immunoglobulin genes) status, CLL or MBL sample, epigenetic subtype (epitype: naive-like, n-CLL; intermediate, i-CLL; memory-like, m-CLL), sequencing data type; prior treatment, U1 and IGLV3-21^{R110} mutations—black. Asterisks—discovery by CLUMPS. Bottom tracks—Lower frequency sSNV/indels and sCNAs, designated as novel, known events or both. Bottom boxed inset—candidate driver genes, frequency <1%. FIG. 1D provides a plot showing representative genes identified by CLUMPS. 3D protein structure of MAP2K2 and DIS3. Mutated residues clustered in functional regions. FIG. 1E provides a plot showing recurrent copy number gains (top) and losses (bottom) by GISTIC analysis showing arm level (left) and focal events (right). Chromosome number—vertical axis; dashed line—significance, q=0.1. Blacklisted regions—gray. Arm level events are labeled with cytoband and frequency (n=984). Focal events denote cytoband, frequency, number of genes encompassed in peak (bracketed), and genes of interest. Shaded font: novel focal events with frequency >2%. Dark font: previously known events.

FIGS. 2A-2F provide plots, a bar graph, and heat maps demonstrating that M-CLL (CLL with mutated IGHV) and U-CLL (CLL with unmutated IGHV) have unique genomic landscapes. FIGS. 2A and 2B provide plots showing a comparison of candidate driver genes (FIG. 2A) or copy number gains/losses (up/down triangle, respectively, FIG. 2B) in U-CLL (CLL with unmutated IGHV) (y-axis, WES, n=459) vs. M-CLL (CLL with mutated IGHV) (x-axis, WES, n=512) plotted by −log₁₀ (q-value). Significance—dashed line. Representative candidate drivers are annotated. Frequency in entire cohort (n=984)—size of circle (FIG. 2A) or triangle (FIG. 2B). drivers predominantly in U-CLL (CLL with unmutated IGHV) generally cluster in the upper and left portions of the plots; drivers predominantly in M-CLL (CLL with mutated IGHV) generally occur in the lower and right portions of the plots. FIG. 2C provides plots showing league model timing diagrams comparing acquisition of somatic mutation and arm level somatic copy number alterations (sCNAs) in M-CLL (CLL with mutated IGHV) (top, n=251) and U-CLL (CLL with unmutated IGHV) (bottom, n=354). Higher timing score (x-axis) denotes later event; median scores—vertical light grey marks (95% confidence interval, gray). MYD88, tri(19), DIS3, ITPKB, DICER1, CARD11—events significant in M-CLL; lighter shaded text in lower panel of FIG. 2C—events significant in U-CLL; black text—events shared by M-CLL (CLL with mutated IGHV) and U-CLL. Asterisks—significant difference in timing (q<0.1). FIGS. 2D and 2E provide heat maps showing somatic alterations associated with failure free survival (FFS) and overall survival (OS) in M-CLL (CLL with mutated IGHV) (FIG. 2D, WES/WGS, n=519) and U-CLL (CLL with unmutated IGHV) (FIG. 2E, WES/WGS, n=476). Events ranked by elastic net (ENET) coefficients, which identifies variables to be included in the model, shrinking coefficients to 0 when excluded. Heatmap denotes hazard ratios (HR) for ENET and univariate Cox regressions. Events included by ENET model (concentric circle) or significant in univariate analysis only (closed circle) in treatment-naive, non-trial patients (M-CLL, n=394; U-CLL, n=247) annotated on right. Lighter text—novel alterations (see Tables 1 and 2). FIG. 2F provides a heat map showing number of candidate drivers in three genomic analyses: entire cohort (All), M-CLL (CLL with mutated IGHV) and U-CLL. Union—intersection of these analyses and total putative drivers identified.

FIGS. 3A-3D provide plots, heat maps, bar graphs, a dendogram, and a boxplots showing CLL subtypes based on epigenetic and transcriptomic features. FIG. 3A provides a plot showing the main sources of variability in the DNA methylome were epitype and epiCMIT as determined by unsupervised principal component analysis in samples analyzed by 450k methylation array (top, n=490) or single-end reduced representation bisulfite sequencing (RRBS-SE, bottom, n=388). FIG. 3B provides heat maps and bar graphs showing eight gene expression clusters (ECs, columns) identified by Bayesian non-negative matrix factorization (BNMF) method in 610 treatment-naive samples. Heatmap demonstrated associated upregulated (top portion of lower heat map) and downregulated (lower portion of lower heat map) marker genes for each cluster (rows) with select genes (right, see Tables 3 and 4). Right vertical panel demonstrated upregulated or downregulated histone 3 lysine 27 acetylation (H3K27ac) in regulatory regions for each marker gene; EC-o and EC-i H3K27ac was not assessed due to low sample size (NA, gray). Header—number of samples in ECs; association with IGHV subtype (M-CLL; U-CLL); epitype (n-CLL; i-CLL; m-CLL). Frequency of common CLL alterations is shown for each EC. Significant associations—asterisks (q<0.1, curveball algorithm). FIG. 3C provides a dendrogram of expression clusters (ECs) with associated upregulated and downregulated biologic pathways determined by gene set enrichment analysis (see FIG. 14B). FIG. 3D provides boxplots showing cellular proliferative history, represented by epiCMIT, varied in expression clusters (ECs) enriched with M-CLL (CLL with mutated IGHV) epitype. EC-m3 had significantly lower epiCMIT relative to EC-m1, EC-m2, and EC-m4 (p-values by t-test). Boxplots: center line, median; box limits, upper and lower quartiles; whiskers, 1.5× interquartile range.

FIGS. 4A-4E provide plots and heat maps demonstrating that expression clusters and integrated analysis predicts clinical outcome. FIGS. 4A and 4B provide plots showing a Kaplan Meier analysis of the impact of expression clusters on (FIG. 4A) failure free survival (FFS) and (FIG. 4B) overall survival (OS) probabilities in 609 treatment-naive samples (log-rank test). FIG. 4C provides a plot showing a Kaplan Meier analysis assessing the difference in failure free survival (FFS) probability between samples with concordant IGHV (heavy chain variable region of immunoglobulin genes) status and expression clusters (ECs) (e.g., M-CLLs in EC-m clusters) versus those that are discordant (e.g., M-CLLs in EC-u clusters). M-CLLs—left; U-CLLs (CLLs with unmutated IGHV)—right. FIGS. 4D and 4E provide heat maps showing genetic, epigenetic, and transcriptomic features associated with (FIG. 4D) failure free survival (FFS) and (FIG. 4E) overall survival (OS) in treatment-naive samples (n=506). Events ranked by elastic net (ENET) coefficients, which identifies variables to be included in the model, shrinking coefficients to 0 when excluded. Heatmap denotes hazard ratios (HR) for ENET and univariate Cox regressions (see Tables 5 and 6). Continuous variable—Φ) (epiCMIT).

FIG. 5 provides plots showing mutational signatures identified as artifacts in whole genome sequencing (WGS) of 177 CLLs.

FIGS. 6A-6E present bar graphs, mutation maps, a Venn diagram, and protein structures showing a dataset description and representative driver gene maps. FIG. 6A provides a bar graph showing the full dataset (n=1156), with contributions by cohort and data type delineated. FIG. 6B provides a Venn diagram showing numbers of samples with genomic, epigenomic, and transcriptomic data. FIG. 6C shows 3D protein structures of representative genes identified by CLUMPS in pan-CLL analysis (n=984). Mutated residues are labeled. Not being bound by theory, a peptide from RAF1 (designated at bottom-center, in complex with 14-3-3 zeta) shows clustered mutations around S259, whose phosphorylation regulates RAF1 activity and is a cancer mutational hotspot that, when mutated, perturbs the interaction with the 14-3-3 zeta and upregulates RAF1 kinase activity. In DICER1, mutations occur in the RNase III domain, including the cancer hotspot residue E1813. Not being bound by theory, this region is critical for Mg²⁺ binding and is required for ribonuclease activity to process microRNAs and mediate post-transcriptional gene regulation. RPS23 mutations are clustered in a conserved loop of the ribosomal decoding center, surrounding P62, whose post-translational hydroxylation affects translation termination accuracy. These RPS23 mutations had a median CCF >80% (FIG. 11D). FIG. 6D shows individual mutations maps of selected novel, putative driver genes. Mutation subtype and position are shown. FIG. 6E shows proteins structures for selected genes identified by CLUMPS in IGHV (heavy chain variable region of immunoglobulin genes) subtypes; mutated residues are labeled. Although BRAF was not identified as a potential M-CLL (CLL with mutated IGHV) driver via MutSig2CV (see FIGS. 8A and 8B), CLUMPS revealed three mutated sites clustered in the kinase domain that are cancer hotspots, thus confirming BRAF as a shared driver (left). Mutated residues in BRAF in U-CLL (CLL with unmutated IGHV) (bottom) are shown for comparison, revealing a greater number of clustered mutations relative to M-CLL. In U-CLL, novel mutations were found in RRM1 (right). Somatic alterations were clustered in the N-terminal ATP-binding site and therefore have potential to impact enzymatic activity.

FIGS. 7A and 7B present a schematic and a stacked barplot showing CLL biological pathways affected by candidate driver genes. FIG. 7A provides a schematic of CLL pathways containing previously identified (black text) and novel (grey text) putative driver genes. Novel drivers clustered in central processes driving CLL (e.g., DNA damage, chromatin modification, RNA processing), but also highlight new pathways not previously implicated by driver genes (e.g., cytoskeleton and extracellular matrix, proteostasis, metabolism). Asterisks—mutated genes discovered by CLUMPs. FIG. 7B provides a stacked barplot ranked by the number of candidate driver genes per CLL pathway. Shaded bars show the number of newly identified (i.e., novel) drivers in each pathway.

FIGS. 8A and 8B provide genomic landscape diagrams showing candidate driver alterations discovered in IGHV subtypes. FIGS. 8A and 8B provide genomic landscape diagrams showing the landscape of putative driver genes and somatic copy number alterations (sCNAs) in M-CLL (CLL with mutated IGHV) (a, n=512) and U-CLL (CLL with unmutated IGHV) (b, n=459) with associated frequencies (rows, barplots). Header tracks annotate cohort, IGHV (heavy chain variable region of immunoglobulin genes) status (M-CLL; U-CLL), disease type (CLL; MBL), epitype (n-CLL; i-CLL; m-CLL), datatype (WES; WGS; both); prior treatment, U1 and IGLV3-21R110 mutations are annotated in black text; grey text—novel alterations; asterisks—discovery by CLUMPS.

FIGS. 9A and 9B provide plots showing chromosomal gains and losses identified in M-CLL (CLL with mutated IGHV) and U-CLL. FIGS. 9A and 9B provide plots showing recurrent copy number gains (left) and losses (right) by GISTIC analysis showing arm level (left per plot) and focal events (right per plot) in M-CLL (CLL with mutated IGHV) (FIG. 9A, n=512) and U-CLL (CLL with unmutated IGHV) (FIG. 9B, n=459). Chromosomes are labeled along the vertical axis; dashed line—significance at q=0.1. Blacklisted regions are colored gray. All arm level events are labeled with cytoband arm and frequency in cohort. Focal events are annotated by cytoband, frequency, number of genes encompassed in peak (bracketed), and genes of interest. Novel focal events with frequency >2% are labeled to the right of each plot and further include 3p 1.5% (novel focal events are also labeled in grey font). Black font: previously identified events.

FIGS. 10A-10C provide a circus plot, a schema, and a barplot showing the landscape of driver alterations and chromosomal aberrations in IGHV (heavy chain variable region of immunoglobulin genes) subtypes. FIG. 10A provides a circos plot summarizing the genomic landscape of CLL IGHV (heavy chain variable region of immunoglobulin genes) subtypes. The identified 97 driver genes, U1 and IGLV3-21^{R110} mutations are labeled according to their genomic location (outside ring, numbered by chromosome). The tracks show the frequency of each candidate driver gene in M-CLL (CLL with mutated IGHV) vs. U-CLL (CLL with unmutated IGHV) (track 1; outermost), location and frequency of the 91 focal somatic copy number alterations (sCNAs)(track 2; gains; losses), and density of SV breakpoints of deletions (track 3) and translocations (track 4) (M-CLL n=88; U-CLL (CLL with unmutated IGHV) n=87; WGS, windows of 1-Mb). The innermost plot highlights translocations in which either one or both breakpoints are recurrent in at least 3 cases (windows of 1-Mb considered to define recurrence) in M-CLL and U-CLL (CLL with unmutated IGHV) with most recurrent breakpoint occurring at the somatic copy number alteration (sCNA) loci 13q14.3. Deletions, inversions, and tandem duplications where both breakpoints were found in at least 2 cases and did not overlap with a driver somatic copy number alteration (sCNA) are shown. Note that only a focal deletion in SP140 found in two U-CLL (CLL with unmutated IGHV) cases met this criterion. FIG. 10B provides a schema of the translocation t(14;18) [IG-BCL2] and deletion of 14q24.1-q32.32 [IGH-ZFP36L1] in the whole genome sequencing (WGS) cohort. All 5 BCL2 translocations were found in M-CLL with IG breakpoints in the J and D genes suggesting that an aberrant V(D)J recombination caused these events. In contrast, 4 U-CLL (CLL with unmutated IGHV) cases (all with 100% IGHV identity) carried the IGH-ZFP36L1 deletion, which truncates the latter gene. In all four cases, the alteration seemed to be mediated by the class-switch recombination machinery since the IGH breakpoints occurred in the class-switch regions. FIG. 10C presents a barplot summarizing the number, recurrence and mechanism behind the Immunoglobulin (IG) SVs observed in the whole genome sequencing (WGS) and whole exome sequencing (WES) cohorts. Similar to whole genome sequencing (WGS) results, most (n=9 of 10) BCL2 translocations detected in whole exome sequencing (WES) were found in M-CLL and mediated by an aberrant V(D)J recombination either in the IGH (n=7) or IGK (n=2) loci. The sole BCL2 translocation observed in U-CLL (CLL with unmutated IGHV) (100% IGHV identity) was due to aberrant class-switch recombination. One IGH-ZFP36L1 deletion was observed in a case with “unknown” IGHV (heavy chain variable region of immunoglobulin genes) status since two populations were detected (one U-CLL (CLL with unmutated IGHV) and one M-CLL), although the major population was unmutated with a 100% IGHV (heavy chain variable region of immunoglobulin genes) identity. In this case, this alteration also occurred in a class-switch recombination region. Note that the detection of IGH-ZFP36L1 deletions in whole exome sequencing (WES) is impaired by the low number of sequencing reads in the class-switch regions. In WES, U-CLLs (CLLs with unmutated IGHV) carried a higher number of non-recurrent Ig events than M-CLL.

FIGS. 11A-11D provide plots showing mutational mechanisms and cancer cell fractions of candidate drivers. FIG. 11A provides barplots showing eight mutational signatures identified in 177 WGS. 3 signatures corresponded to known artifacts and were therefore excluded (see “Mutational signatures review” below). The barplots are labeled with single-base substitution (SBS) number and identity (per COSMIC v3.1), and demonstrate mutation contribution for each of the 5 signatures. FIG. 11B provides a comparison omparison of the normalized signature intensity of the mutational signatures in U-CLL (CLL with unmutated IGHV) (right) vs. M-CLL (left). The nc-AID and c-AID 1 signatures were enriched in M-CLL, whereas the aging signature was more prevalent in U-CLL. There was a trend of increased mutations due to the c-AID 2 signature in U-CLL. All p-values were calculated with Wilcoxon rank-sum test. Boxplots: center line, median; box limits, upper and lower quartiles; whiskers, 1.5× interquartile range; points, outliers. FIG. 11C provides a plot showing proportions of clustered mutations contributed by the two c-AID related signatures (SBS84, c-AID 1 vs. SBS85, c-AID 2) for each IGHV (heavy chain variable region of immunoglobulin genes) subtype (M-CLL; U-CLL) FIG. 11D provides boxplots showing mean cancer cell fraction (CCF) for each non-silent mutation across all candidate driver genes identified in whole exome sequencing (WES) samples (n=984). Color of dots depicts the IGHV (heavy chain variable region of immunoglobulin genes) subtype (M-CLL, dark grey; U-CLL, light grey). The horizontal red line is the threshold for clonality (CCF>85%). Grey text labels—newly identified putative driver genes. The number of non-silent mutations per driver gene is shown at the bottom. Boxplots: center line, median; box limits, upper and lower quartiles; whiskers, 1.5× interquartile range.

FIGS. 12A-12F provide consensus clustering matrices, heatmaps, plots, and an schematic showing development and validation of epitype assignment and epiCMIT in RRBS data. FIG. 12A provides consensus clustering matrices for K=3 groups for RRBS-PE (n=136) and RRBS-SE (n=388) data. Samples present in DNA methylation consensus matrices for RRBS-PE and RRBS-SE were used to perform consensus clustering with 153 and 32 CpGs (FIG. 12D). FIG. 12B presents plots showing empirical cumulative distributions (CDF) to the entries of consensus matrices for K=2 to K=7 for RRBS-PE and RRBS-SE data. FIG. 12C provides plots showing relative change under the cumulative distribution function (CDF) for K=2 to K=7 for RRBS-PE and RRBS-SE data. FIG. 12D provides heatmaps of the CpGs used for consensus clustering of RRBS-PE (153 CpGs) and RRBS-SE (32 CpGs) in FIG. 12A. Each sample (columns) is annotated in header tracks by epitype max probability, IGHV (heavy chain variable region of immunoglobulin genes) status (M-CLL; U-CLL), IGHV (heavy chain variable region of immunoglobulin genes) percent identity, and presence of IGLV3-21^{R110} mutation. FIG. 12E provides a schematic showing the development of the new epiCMIT methodology for RRBS data. First, the genome was segmented into Chromatin Hidden Markov Model (CHMM) (Ernst, J. & Kellis, M. ChromHMM: automating chromatin-state discovery and characterization. Nat. Methods 9, 215-216 (2012)) states using ChIP-seq data to get repressed chromatin regions, where differential DNA methylation analyses was performed in high coverage whole-genome bisulfite sequencing (WGBS) data between the cells with the lowest and highest accumulated cell divisions in the B-cell lineage, namely the hematopoietic precursor cells (HPC) and bone-marrow plasma cells (bmPC). Only CPGs showing extensive differences were retained and constituted the epiCMIT-hyper CpGs or epiCMIT-hypo CpGs depending whether they gain or lose DNA methylation from <0.1 to ≥0.5 and from >0.9 to ≤0.5 from HPC to bmPC, respectively. Finally, epiCMIT-hyper and epiCMIT-hypo scores were calculated according to the available epiCMIT-CpGs per sample, and the higher score in each sample was then selected. FIG. 12F provides a plot showing epiCMIT values on the same samples profiled twice with different platforms. Approach 1—profiled with Illumina-450k (green); approach 2—profiled with RRBS-PE (violet). In samples profiled with Illumina 450k, the original epiCMIT-CpGs were used (Duran-Ferrer, M. et al. The proliferative history shapes the DNA methylome of B-cell tumors and predicts clinical outcome. Nature Cancer 1, 1066-1081 (2020)), whereas in samples profiled with RRBS, the epiCMIT was calculated in each sample with all available epiCMIT-CpGs for the new catalogue (FIG. 12E).

FIGS. 13A-13J provide stacked barplots, a consensus matrix, heatmaps, a plot, a barplot, and boxplots showing identification of expression clusters with associated biologic features. FIG. 13A provides a stacked barplot showing the distribution of individual cohorts in each expression cluster. FIG. 13B provides a consensus matrix for RNA expression profiles of 610 treatment-naive CLLs by repeated hierarchical clustering with 80% resampling and varying cutoffs for number of clusters. This matrix served as input to the Bayesian non-negative matrix factorization (BayesNMF) method for inferring the total number of clusters and sample assignment to clusters. FIG. 13C provides a plot showing uniform manifold approximation and projection (UMAP) showing clustering of expression clusters (ECs) (n=610). The EC-u clusters are near the top, EC-m and EC-o in the middle, and EC-i at the bottom. Analysis was performed using the marker genes identified by BayesNMF. FIG. 13D provides a boxplot showing a comparison of the percent IGHV (heavy chain variable region of immunoglobulin genes) identity among the ECs. Dotted line notes the 98% threshold defining M-CLL and U-CLL. EC-i and EC-o displayed borderline IGHV (heavy chain variable region of immunoglobulin genes) identity near 98% which was significantly different from the EC-u and EC-m clusters. All p-values were calculated using two-sided t-tests. Boxplots: center line, median; box limits, upper and lower quartiles; whiskers, 1.5× interquartile range. FIG. 13E provides boxplots showing a comparison of the percent IGHV (heavy chain variable region of immunoglobulin genes) identity between those samples with concordant IGHV (heavy chain variable region of immunoglobulin genes) status and expression clusters (ECs) (e.g., M-CLLs in EC-m clusters) versus the discordant samples (e.g., M-CLLs in EC-u clusters). IGHV (heavy chain variable region of immunoglobulin genes) mutated cases—left; IGHV (heavy chain variable region of immunoglobulin genes) unmutated samples—right. There was a small difference in mean percent identity in U-CLLs (CLLs with unmutated IGHV), but no difference among M-CLL cases. All p-values were calculated using two-sided t-tests. Boxplots: center line, median; box limits, upper and lower quartiles; whiskers, 1.5× interquartile range. FIG. 13F provides a barplot showing percentage of cases carrying stereotyped immunoglobulin genes within each EC. Red horizontal line represents the percentage of stereotyped cases in the whole cohort. FIG. 13G provides a stacked barplot showing fraction of cases classified in each CLL stereotype subset according to their EC. FIG. 13H provides stacked barplots showing percentage of IGHV (heavy chain variable region of immunoglobulin genes) (left) and IG(K/L)V (right) gene usage within each EC. IGKV genes from proximal and distal clusters were merged for simplification. All p-values were calculated using Chi-squared tests corrected by the Benjamini-Hochberg procedure (q-values, q). q<0.1; *, q<0.05; **, q<0.001; ***, q<0.0001. FIGS. 13I and 13J provide heatmaps showing upregulated (FIG. 13I) and downregulated (FIG. 13J) histone 3 lysine 27acetylation (H3K27ac) in regulatory regions of expression cluster (EC) marker genes. The genomic coordinates of expression cluster (EC) marker genes were selected with an additional 2,000 bp upstream of their respective transcription start sites, to ensure capture of regulatory regions of the respective genes. These coordinates were then intersected with the H3K27ac consensus matrix showing at least 1 H3K27ac peak in 1 CLL sample to perform differential H3K27ac levels among ECs. Throughout the figures, the order from top to bottom of the elements represented in each stacked bar in the barplots corresponds to the order of the elements listed in the legend read from top-to-bottom and from left-to-right.

FIGS. 14A-14J present plots, confusion matrices, boxplots, and a stacked barplot showing expression cluster (EC) differential gene expression, pathway activity, and classifier. FIG. 14A provides plots showing differentially expressed genes (grey) by comparison of each expression cluster (EC) versus all other samples (n=610 total) using limma-voom (Ritchie, M. E. et al. limma powers differential expression analyses for RNA-sequencing and microarray studies. Nucleic Acids Res. 43, e47 (2015)). Marker genes identified by the BayesNMF method are identified in the plot as dark grey points falling outside of the area within and below the dashed lines. ChIP-seq data for H3K27ac, which marks actively expressed chromatin, was available for a subset of samples (n=70). Significant up- or down-regulation of H3K27ac marks are denoted by up and down pointing triangles, respectively. Of note, due to low sample size, differential H3K27ac levels were not evaluated for EC-o and EC-i. FIG. 14B provides plots showing a gene set enrichment analysis identifying upregulated and downregulated pathways in each EC. Normalized enrichment scores (NES) are plotted on the x-axis. The large diamond denotes the NES for the designated expression cluster (EC) compared to all other expression clusters (ECs) (circles). FIG. 14C provides a confusion matrix for the expression cluster (EC) classifier portraying the accuracy of the classifier on the test set per EC. The “Dominance” score used for coloring was computed by normalizing each value in the confusion matrix by the sum of its respective row and column. FIG. 14D provides a boxplot showing confidence in correctly classified samples is greater than for incorrectly classified samples (two-sided t-test). The “prediction margin” was computed by the difference in percentage of RandomForest votes for the top class and the class with the 2nd to most votes. Boxplots: center line, median; box limits, upper and lower quartiles; whiskers, 1.5× interquartile range. FIG. 14E provides a plot showing a receiver-operator curve (ROC) using the prediction margin to find an optimal cutoff (0.328) with the Youden's J statistic (J=Sensitivity+Specificity−1), which achieves 0.962 specificity and 0.615 sensitivity. FIGS. 14F-14H are the same as c-e, but using batch-corrected transcripts Per Million (TPMs), which improves classifier accuracy from 79% to 83%. FIG. 14I provides a stacked barplot comparing the expression cluster (EC) distributions in the discovery cohort by BayesNMF to the predictions made by the classifier for the discovery cohort (n=610), an extension cohort of samples not included the discovery cohort (n=110), and an external CLL cohort (n=136) (Dietrich, S. et al. Drug-perturbation-based stratification of blood cancer. J. Clin. Invest. 128, 427-445 (2018)). FIG. 14J provides plots showing stability of the expression clusters (ECs) over time in longitudinally sampled CLL samples (Gruber, M. et al. Growth dynamics in naturally progressing chronic lymphocytic leukaemia. Nature 570, 474-479 (2019)). Timepoints are noted on the x-axis with the number of years between the first and last sample listed above the curves. Throughout the figures, the order from top to bottom of the elements represented in each stacked bar in the barplots corresponds to the order of the elements listed in the legend read from top-to-bottom and from left-to-right.

### DETAILED DESCRIPTION OF THE INVENTION

The invention features compositions, panels of biomarkers, and methods that are useful for characterizing chronic lymphocytic leukemia (CLL) for prognosis and selection of a subject for a treatment and/or inclusion in a clinical trial.

The invention is based, at least in part, upon the discovery of eight new chronic lymphocytic leukemia (CLL) gene expression subtypes and their efficacy in guiding prognosis and selection of subjects for a treatment. Not being bound by theory, the gene expression subtypes correspond to gene expression clusters enriched with unique genetic and epigenetic features, distinguished by cellular pathways, and useful as an independent prognostic factor. A machine classifier was developed, as described further in the Examples provided herein, that can effectively classify a chronic lymphocytic leukemia (CLL) as belonging to a particular gene expression subtype associated with a corresponding gene expression cluster. The gene expression clusters and their corresponding expression subtypes are termed Ec-i, EC-m1, EC-m2, EC-m3, EC-m4, EC-o, EC-u1, and EC-u2. In embodiments, the gene expression subtype is used in combination with genetic drivers and epigenetic states in a prognostic model.

Previous analysis of chronic lymphocytic leukemia (CLL) have provided only fragments of the ‘CLL map’, each focusing on particular patient populations or different data types, but none have built a comprehensive atlas with sufficient power and resolution to fully characterize the whole bioclinical spectrum of the disease. As described in the Examples provided herein, this challenge was addressed through an integrated genomic, transcriptomic, and epigenomic analysis of data from 1156 patients. 202 candidate genetic drivers of CLL (109 novel) were identified and allowed for refining the characterization of CLL IGHV (heavy chain variable region of immunoglobulin genes) subtypes, which revealed distinct genetic landscapes with unique patterns of leukemogenic trajectories. Discovery of the new gene expression subtypes further subcategorized this neoplasm and proved to be an independent prognostic factor. Clinical outcomes were associated with a combination of genetic alterations, epigenetic states, and gene expression clusters, further advancing our prognostic paradigm. Overall, the work described in the Examples provided herein provides fresh insights into CLL oncogenesis and prognostication.

### Chronic Lymphocytic Leukemia (CLL)

Chronic lymphocytic leukemia (CLL) is a type of cancer in which the bone marrow makes too many lymphocytes. Early on there are typically no symptoms. Later, non-painful lymph node swelling, feeling tired, fever, night sweats, or weight loss for no clear reason may occur. Enlargement of the spleen and low red blood cells (anemia) may also occur. It typically worsens gradually (i.e., “chronic”) over years.

Chronic lymphocytic leukemia (CLL) is a B cell neoplasm with variable natural history that is conventionally categorized into two major subtypes distinguished by the extent of somatic mutations in the heavy chain variable region of immunoglobulin genes (IGHV).

### Panels

The present disclosure provides panels of biomarkers and the use of such panels for characterizing chronic lymphocytic leukemia (CLL). As would be understood, references herein to a biomarker, a panel of biomarkers, or other similar phrase indicates one or more of the biomarkers listed below, in Tables 3A-3B and 4, or otherwise described herein.

In one embodiment, markers useful in the panels of the invention include, for example, ABCA9, ACAP3, ACSM3, ADAP2, AF127936.7, ARHGAP33, ARMC7, ARRDC5, ARSD, ARSI, ASB2, ATP1A3, ATP2B1, ATPIF1, BASP1, BCL2A1, BCL7A, BCS1L, CAMK2A, CLDN23, CMTM7, COBLL1, CRELD2, CRY1, CTAGE9, CTLA4, DDR1, DKFZP761J1410, DPF3, EML6, ERRFI1, ESPNL, EZH2, FAHD2B, FAM109A, FBXO27, FGL2, FLJ20373, FMOD, GADD45A, GNAO1, GPR160, GPR34, GUCD1, HCK, HDAC4, HIP1R, HMCES, IGSF3, IQSEC1, ITGAX, KCNH3, KCNN3, KCTD3, KDM1B, KLK1, KSR1, LCN10, LINC00865, LPL, LRRK2, LUZP1, MAP4K4, MAPK4, MAST4, MPRIP, MRO, MSI2, MVB12B, MYBL1, MYC, MYL5, MYL9, MYO3A, NEDD9, NFKBIZ, NR2F6, NRIP1, NRSN2, NUGGC, P2RX1, PELI3, PIGB, PIP5K1B, PITPNC1, PLD1, PTPN7, QDPR, REPS2, RHBDF2, RIMKLB, RP11-134N1.2, RP11-265P11.1, RP11-453F18_B.1, RP11-456H18.2, RP1-90J20.12, SAMSN1, SCPEP1, SH3D21, SLC44A1, SLC4A7, SLC4A8, SMIM10, SPN, SSBP3, STAM, STX5, SYNGR3, TAS1R3, TBC1D2B, TBC1D9, TFEC, TIMELESS, TNFRSF13B, TNR, TOX2, TRIM7, TUBG2, VSIG10, WNT5A, ZMYND8, and ZNF804A, or a sub-set thereof, as well as the nucleic acid molecules encoding such proteins. In another embodiment, markers useful in the panels of the invention include, for example, ACAP3, ACSM3, AEBP1, AKT3, ARHGAP33, ARHGAP42, ARMC7, ARRDC5, ATPIF1, BACH2, BASP1, BCL7A, C17orf100, CBLB, CD72, CD86, CEACAM1, CHPT1, CLDN7, CMTM7, CNTNAP1, COBLL1, COL18A1, CRY1, CTLA4, EGR3, EML6, EZH2, FADS3, FCER1G, FCRL2, FGL2, FLJ20373, FMOD, GADD45A, GLIPR1, GNB4, GPR160, GPR34, GRIK3, GUCD1, HCK, HIP1R, HIVEP3, HMCES, IGF2BP3, IGSF3, IL21R, INPP5F, IQGAP2, IQSEC1, ITGAX, ITGB5, JDP2, KANK2, KCNH2, KDM1B, KLF3, LATS2, LCN10, LEF1, LPL, LRRK2, LUZP1, MAP4K4, MID1IP1, MMP14, MPRIP, MSI2, MYBL1, MYL9, MYLIP, MZB1, NBPF3, NRIP1, NRSN2, NUGGC, NXPH4, P2RX1, P2RX5, P2RY14, PDGFD, PIP5K1B, PITPNC1, PON2, PRICKLE1, PTPN7, RCN3, RDX, RHBDF2, RIMKLB, RNF135, RP11-145M9.4, RP11-268J15.5, RP11-463012.3, RP5-1028K7.2, SAMSN1, SCCPDH, SCD, SCPEP1, SDC3, SECTM1, SESN3, SH3BP2, SH3D21, SLC16A5, SLC19A1, SLC4A7, SPN, SSBP3, STX5, SUSD1, TBC1D2B, TBC1D9, TBKBP1, TCF7, TFEC, TGFBR3, TIGIT, TIMELESS, TMEM133, TNFRSF13B, TOX2, TRAK2, TTC39C, TUBG2, VPS37B, VSIG10, WNT9A, ZAP70, ZNF667-AS1, ZNF804A, and ZSWIM6, or a subset thereof, as well as the nucleic acid molecules encoding such proteins. Fragments of the aforementioned polypeptides useful in the methods of the invention are sufficient to bind an antibody that specifically recognizes the protein from which the fragment is derived.

In embodiments, markers useful in the panels of the invention include markers for expression cluster Ec-i, namely, GRIK3, IQGAP2, FCER1G, STK32B, GADD45A, ITGAX, KLF3, RFTN1, PTK2, DFNB31, and ZMAT1, or a sub-set thereof, as well as the nucleic acid molecules encoding such proteins. In embodiments, markers useful in the panels of the invention include markers for expression cluster EC-m1, namely, TFEC, COL18A1, SLC19A1, NRIP1, KCNH2, P2RX1, ARRDC5, BEX4, and APP, or a sub-set thereof, as well as the nucleic acid molecules encoding such proteins. In embodiments, markers useful in the panels of the invention include markers for expression cluster EC-m2, namely, EML6, HCK, CD1C, VPS37B, CYBB, NXPH4, BTNL9, KLRK1, IQSEC1, BANK1, LEF1, SH3D21, FMOD, SEMA4A, CTLA4, ADTRP, IGSF3, IGFBP4, PDGFD, and APOD, or a sub-set thereof, as well as the nucleic acid molecules encoding such proteins. In embodiments, markers useful in the panels of the invention include markers for expression cluster EC-m3, namely, MS4A4E, MYL9, NT5E, MS4A6A, PITPNC1, CNTNAP2, IGF2BP3, WNT3, CLDN7, TCF7, BASP1, F1120373, MAP4K4, LRRK2, SAMSN1, CEACAM1, TNFRSF13B, PHF16, MID1IP1, and ABCA9, or a sub-set thereof, as well as the nucleic acid molecules encoding such proteins. In embodiments, markers useful in the panels of the invention include markers for expression cluster EC-m4, namely, MYBL1, NUGGC, GNG8, AEBP1, HIP1R, LATS2, RIMKLB, EML6, FADS3, MBOAT1, LCN10, DCLK2, and GLUL, or a sub-set thereof, as well as the nucleic acid molecules encoding such proteins. In embodiments, markers useful in the panels of the invention include markers for expression cluster EC-o, namely, ACSM3, TOX2, PHF16, SESN3, TBC1D9, PIP5K1B, SIK1, DUSP5, GNG7, HIVEP3, MARCKSL1, GPR183, HRK, and PITPNC1, or a sub-set thereof, as well as the nucleic acid molecules encoding such proteins. In embodiments, markers useful in the panels of the invention include markers for expression cluster EC-u1, namely, SEPT10, LDOC1, LPL, KANK2, SOWAHC, DUSP26, OSBPL5, WNT9A, FGFR1, GTSF1L, ADD3, AKT3, COBLL1, MNDA, FCRL3, FAM49A, FCRL2, SLC2A3, and MARCKS, or a sub-set thereof, as well as the nucleic acid molecules encoding such proteins. In embodiments, markers useful in the panels of the invention include markers for expression cluster EC-u2, namely, ITGB5, BCL7A, PPP1R9A, TSPAN13, SLC12A7, SSBP3, VASH1, SPG20, IL13RA1, NR3C2, TUBG2, ZNF804A, and IL2RA, or a sub-set thereof, as well as the nucleic acid molecules encoding such proteins. The panels can comprise biomarkers for expression cluster Ec-i, EC-m1, EC-m2, EC-m3, EC-m4, EC-o, EC-u1, or EC-u2, or various combinations thereof.

The invention further features the use of such panels for characterizing chronic lymphocytic leukemia (CLL). In embodiments, the panels are used in combination with a classifier (e.g., a machine learning classifier) to identify a CLL as belonging to a particular expression subtype. The panels are advantageously used for guiding selection of a subject for a CLL treatment.

### Biomarkers

Measurements of expression levels of biomarkers (e.g., polypeptide and/or polynucleotides encoding polypeptides present in expression clusters described herein) are used in combination with a model (e.g., a machine learning classifier) to identify a chronic lymphocytic leukemia as belonging to a particular expression subtype. In particular embodiments, a biomarker is an organic biomolecule that is differentially present in a sample taken from a subject of one phenotypic status (e.g., having a disease, such as chronic lymphocytic leukemia (CLL)) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease (e.g., chronic lymphocytic leukemia (CLL)).

A biomarker of the invention may be detected in a biological sample of the subject (e.g., tissue, fluid), including, but not limited to blood, blood serum, plasma, saliva, urine, ascites, cyst fluid, a homogenized tissue sample (e.g., a tissue sample obtained by biopsy), a cell isolated from a patient sample, and the like.

The invention provides panels comprising isolated biomarkers. The biomarkers can be isolated from biological fluids. They can be isolated by any method known in the art. In certain embodiments, this isolation is accomplished using the mass and/or binding characteristics of the markers. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography. In some embodiments, biomarkers described herein are fixed to a substrate (e.g., chips, beads, microfluidic platforms, membranes).

### Detection of Biomarkers

The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are describe below.

Detection by Sequencing and/or Probes

In particular embodiments, the biomarkers of the invention are measured by a sequencing- and/or probe-based technique (e.g., RNA-seq).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24;109(4):1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Detecting a target polynucleotide sequence or fragment thereof associated with a biomarker that hybridizes to a probe sequence may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various detection methods may be used and are described as follows.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a biomarker.

Detection of the expression level of a biomarker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

In embodiments, levels of biomarkers in a sample are quantified using targeted sequencing. Methods for targeted sequencing are well known in the art (see, e.g., Rehm, “Disease-targeted sequencing: a cornerstone in the clinic”, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, β-galactosidase, urease, alkaline phosphatase or peroxidase, avidin/biotin complex.

Methods used to detect or quantify binding of a probe to a target biomarker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5'S″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the invention.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In embodiments, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In embodiments, a sample is analyzed by means of a protein biochip (also known as a protein microarray). Such biochips are useful in high-throughput low-cost screens to identify alterations in the expression or post-translation modification of a biomarker, or a fragment thereof. In embodiments, a protein biochip of the invention binds a biomarker present in a sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, Calif.), Zyomyx (Hayward, Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions include, as non-limiting examples, temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the biomarkers of this invention are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MS' analysis to be performed. Tandem mass spectrometry allows multiple reactions to be monitored at the same time.

Capillary infusion may be employed to introduce the biomarker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Classification Algorithms

The present invention provides methods for characterizing a chronic lymphocytic leukemia (CLL) as belonging to an expression subtype (e.g., Ec-i, EC-m1, EC-m2, EC-m3, EC-m4, EC-o, EC-u1, and EC-u2). The expression subtype is useful in predicting clinical outcome for a CLL patient and/or for guiding therapy.

In some embodiments, data derived from the assays for detection of biomarkers (e.g., RNA-seq) that are generated using samples such as “known samples” can then be used to “train” a classification model. Exemplary methods for developing a model for classifying a chronic lymphocytic leukemia as belonging to an expression subtype are described in the Examples provided herein. A “known sample” is a sample that has been pre-classified. The data used to form the classification model can be referred to as a “training data set.” Once trained, the classification model (e.g., a machine learning classifier) can be used to classify the expression subtype of a chronic lymphocytic leukemia (CLL) based upon levels of biomarkers detected in a sample. The sample can be taken from a subject having CLL. This can be useful, for example, in guiding selection of a treatment for a subject or for prognostic purposes.

The training data set that is used to form the classification model may comprise raw data or pre-processed data. In embodiments, a classifier can be trained using a random forest classifier, as described in the Examples provided herein.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from a device that is used to detect biomarkers, or it may be coupled to the device.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### Selection of Subjects for Treatment

Panels comprising biomarkers of the invention are used to characterize chronic lymphocytic leukemia (CLL) in a subject to select the subject for treatment with an agent, for prognosis, and/or to characterize the CLL as belonging to an expression subtype (e.g., Ec-i, EC-m1, EC-m2, EC-m3, EC-m4, EC-o, EC-u1, and/or EC-u2). The panels of the invention are used in combination with a classification model, as described in the Examples provided herein, to categorize a chronic lymphocytic leukemia as belonging to an expression subtype selected from Ec-i, EC-m1, EC-m2, EC-m3, EC-m4, EC-o, EC-u1, and EC-u2. In certain embodiments, panels of the invention are used to select a treatment for the subject. In some embodiments, panels of the invention are used to select a subject for inclusion in a clinical study; for example, a subject is selected for treatment if the subject has a CLL of an expression subtype associated with a positive response to a drug being evaluated in the clinical study. In embodiments, the expression subtype is used as an input to an integrated model for predicting a clinical outcome for a subject having CLL. The integrated model can include as inputs, expression subtype, genetic drivers, and epigenetic states.

The invention provides methods for using the expression subtype of a chronic lymphocytic leukemia (CLL) to predict the sensitivity or resistance of a CLL to a drug. The invention further provides methods for selecting a subject with chronic lymphocytic leukemia (CLL) for treatment with a drug to which the CLL is predicted to be sensitive. The invention also provides methods for selecting subjects having chronic lymphocytic leukemia for inclusion in a clinical trial or other drug study where subjects with CLL predicted to be sensitive to a drug being studied in the trial or study are included in the trial or study and/or subjects with CLL predicted to be resistant to the drug are excluded from the trial or study. Tables 7A and 7B provide drug sensitivity and drug resistance information for CLL's having one of the expression subtypes Ec-i, EC-m1, EC-m2, EC-m3, EC-m4, EC-o, EC-u1, and EC-u2.

Based on their expression subtype, subjects are selected for treatment with one or more of the following agents: actinomycin D, afatinib, AT13387, AZD7762, BAY 11-7085, BX912, CCT241533, cephaeline, chaetoglobosin A, dasatinib, duvelisib, everolimus, fludarabine, ibrutinib, idelalisib, KU-60019, KX2-391, MIS-43, MK-1775, MK-2206, navitoclax, NU7441, PF 477736, PRT062607 HCl, rotenone, saracatinib, SD07, selumetinib, SGI-1776, SNS-032, spebrutinib, TAE684, tamatinib, thapsigargin, venetoclax, vorinostat, or YM155. In other embodiments, the drug is AT13387, AZD7762, dasatinib, duvelisib, fludarabine, ibrutinib, idelalisib, navitoclax, PRT062607 HCl, selumetinib, SNS-032, or venetoclax. In some embodiments, based on their expression subtype, subjects are selected for treatment with one or more of the following agents: 1-Ter-Butyl-3-P-Tolyl-1h-Pyrazolo[3,4-D]Pyrimidin-4-Ylamine, 4-HYDROXY-N′-(4-ISOPROPYLBENZYL)BENZOHYDRAZIDE, Amsacrine, Astemizole, Azimilide, Bepridil, Betrixaban, Bosutinib, Carvedilol, Chlorobutanol, Chlorpromazine, Ciprofloxacin, Cisapride, Clarithromycin, Cytarabine, Disopyramide, Dofetilide, Doxepin, Dronedarone, Erythromycin, Flecainide, Fluoxetine, Fluvoxamine, Fostamatinib, Halofantrine, Hydroxyzine, Ibutilide, Imipramine, Isavuconazole, Ketoconazole, Levomefolic acid, Loratadine, Methotrexate, Nefazodone, Nitazoxanide, Pentoxifylline, Pentoxyverine, Perhexiline, Phenytoin, Phosphonotyrosine, Pimozide, Pitolisant, Potassium nitrate, Pralatrexate, Prazosin, Procainamide, Propafenone, Quercetin, Quinidine, See comments, Semaglutide, Sertindole, Sotalol, Tamoxifen, Tecastemizole, Terazosin, Terfenadine, Thioridazine, Topiramate, Trimetrexate, Verapamil, and/or Vernakalant.

In some embodiments, a subject having a CLL with a particular expression subtype is selected for treatment with an agent targeting a gene or polypeptide associated with the expression subtype. In various embodiments, the association of a gene or polypeptide with an expression subtype is determined according to the associations indicated in Table 3A. For example, if the expression subtype is associated with NRIP1, the subject is administered 4-HYDROXY-N′-(4-ISOPROPYLBENZYL)BENZOHYDRAZIDE; if the expression subtype is associated with SLC19A1, the subject is administered one or more of Pralatrexate, Methotrexate, Levomefolic acid, Nitazoxanide, and/or Trimetrexate; if the expression subtype is associated with KCNH2, the subject is administered one or more of Amsacrine, Astemizole, Azimilide, Bepridil, Betrixaban, Carvedilol, Chlorobutanol, Chlorpromazine, Ciprofloxacin, Cisapride, Clarithromycin, Disopyramide, Dofetilide, Doxepin, Dronedarone, Erythromycin, Flecainide, Fluoxetine, Fluvoxamine, Halofantrine, Hydroxyzine, Ibutilide, Imipramine, Isavuconazole, Ketoconazole, Loratadine, Nefazodone, Pentoxyverine, Perhexiline, Phenytoin, Pimozide, Pitolisant, Potassium nitrate, Prazosin, Procainamide, Propafenone, Quinidine, Sertindole, Sotalol, Tamoxifen, Tecastemizole, Terazosin, Terfenadine, Thioridazine, Verapamil, and/or Vernakalant; if the expression subtype is associated with LPL, the subject is administered Semaglutide; if the expression subtype is associated with HCK, the subject is administered one or more of 1-Ter-Butyl-3-P-Tolyl-1h-Pyrazolo[3,4-D]Pyrimidin-4-Ylamine, Phosphonotyrosine, Quercetin, Bosutinib, and/or Fostamatinib; if the expression subtype is associated with NT5E, the subject is administered Pentoxifylline, and/or Cytarabine; and/or if the expression subtype is associated with GRIK3, the subject is administered Topiramate.

In some embodiments, a subject having a CLL determined to have a driver mutation (e.g., a mutation to the DICER1 gene), is administered an agent targeting the gene and/or a product of the gene (e.g., an agent reducing expression or activity of the DICER1 gene and/or polypeptide). In embodiments, the drug sensitivity and drug resistance information provided in Tables 7A and 7B relating to particular drugs and expression subtypes can be extrapolated to apply to those drugs having a similar or the same drug target, targeting the same pathway, belonging to the same drug category, and/or belonging to the same drug group.

The correlation of test results with an expression subtype involves applying a classification algorithm (e.g., a machine learning classifier) of some kind to the results to determine the expression subtype. The classification algorithm may be as simple as determining whether or not the amounts of the markers are above or below a particular cut-off number. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the expression subtype using a computer, e.g., a programmable digital computer. In either case, one can then record the status on tangible medium, for example, in computer-readable format such as a memory drive or disk or simply printed on paper. The result also could be reported on a computer screen.

In one embodiment, this invention provides methods for prognosis. Determining the course of disease can involve determining a probability of survival and/or failure free survival, optionally for or over a particular period of time; for example, about 6 months, 1 yr, 2 yr, 3 yr, 4 yr, 5 yr, 6 yr, 7 yr, 8 yr, 9 yr, 10 yr, 11 yr, 12 yr, 13 yr, 14 yr, 15 yr, 16 yr, 17 yr 18 yr, 19 yr, 20 yr, 21 yr, 22 yr, 23 yr, 24 yr, or 25 yr.

### Hardware and Software

The present invention also provides a computer system useful in analyzing data associated with biomarker expression, patient selection, and related computations (e.g., calculations associated with a machine learning classifier).

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Pharmaceutical Compositions

As reported herein, the panels of biomarkers presented herein can be used in a method to select a subject for treatment with an agent. In embodiments, the treatment is administered as part of a clinical trial. Accordingly, the invention provides chemotherapeutic compositions for treatment of chronic lymphocytic leukemia (CLL). Non-limiting examples of agents suitable for use in the methods provided herein include. The compositions should be sterile and contain a therapeutically effective amount of the polypeptides or nucleic acid molecules in a unit of weight or volume suitable for administration to a subject.

In embodiments, the composition contains a drug selected from actinomycin D, afatinib, AT13387, AZD7762, BAY 11-7085, BX912, CCT241533, cephaeline, chaetoglobosin A, dasatinib, duvelisib, everolimus, fludarabine, ibrutinib, idelalisib, KU-60019, KX2-391, MIS-43, MK-1775, MK-2206, navitoclax, NU7441, PF 477736, PRT062607 HCl, rotenone, saracatinib, SD07, selumetinib, SGI-1776, SNS-032, spebrutinib, TAE684, tamatinib, thapsigargin, venetoclax, vorinostat, or YM155, and the like (e.g., alternative drugs effective in the treatment of chronic lymphocytic leukemia (CLL)). In embodiments, the composition contains a drug selected from AT13387, AZD7762, dasatinib, duvelisib, fludarabine, ibrutinib, idelalisib, navitoclax, PRT062607 HCl, selumetinib, SNS-032, and venetoclax. In embodiments, the drug has the same drug target, targets the same pathway, belongs to the same drug category, and/or belongs to the same drug group as a drug listed in Tables 7A and 7B. In embodiments, the drug has the same drug target, targets the same pathway, belongs to the same drug category, and/or belongs to the same drug group as indicated in Table 7B for AT13387, AZD7762, dasatinib, duvelisib, fludarabine, ibrutinib, idelalisib, navitoclax, PRT062607 HCl, selumetinib, SNS-032, or venetoclax. In some embodiments, the composition contain one or more of the following agents: 1-Ter-Butyl-3-P-Tolyl-1h-Pyrazolo[3,4-D]Pyrimidin-4-Ylamine, 4-HYDROXY-N′-(4-ISOPROPYLBENZYL)BENZOHYDRAZIDE, Amsacrine, Astemizole, Azimilide, Bepridil, Betrixaban, Bosutinib, Carvedilol, Chlorobutanol, Chlorpromazine, Ciprofloxacin, Cisapride, Clarithromycin, Cytarabine, Disopyramide, Dofetilide, Doxepin, Dronedarone, Erythromycin, Flecainide, Fluoxetine, Fluvoxamine, Fostamatinib, Halofantrine, Hydroxyzine, Ibutilide, Imipramine, Isavuconazole, Ketoconazole, Levomefolic acid, Loratadine, Methotrexate, Nefazodone, Nitazoxanide, Pentoxifylline, Pentoxyverine, Perhexiline, Phenytoin, Phosphonotyrosine, Pimozide, Pitolisant, Potassium nitrate, Pralatrexate, Prazosin, Procainamide, Propafenone, Quercetin, Quinidine, See comments, Semaglutide, Sertindole, Sotalol, Tamoxifen, Tecastemizole, Terazosin, Terfenadine, Thioridazine, Topiramate, Trimetrexate, Verapamil, and/or Vernakalant. Agents of the present invention may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agents of the present invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition (e.g., chronic lymphocytic leukemia). The preferred dosage of a nucleobase oligomer of the invention is likely to depend on such variables as the type and extent of the disorder, the overall health status of the particular patient, the formulation of the compound excipients, and its route of administration.

With respect to a subject having chronic lymphocytic leukemia (CLL), an effective amount is sufficient to stabilize, slow, or reduce the proliferation of CLL. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an agent and/or compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Kits

In another aspect, the invention provides kits for aiding in patient selection for treatment and/or characterizing chronic lymphocytic leukemia (e.g., selecting a treatment method for a subject, selection of a subject for a clinical trial, predicting clinical outcome, and the like), which kits are used to detect biomarkers according to the invention. In an embodiment, the kit comprises a drug for use in treatment of chronic lymphocytic leukemia (e.g., fludarabine, ibrutinib, idelalisib, SNS-032, venetoclax, and/or navitoclax). In one embodiment, the kit comprises agents that specifically recognize the biomarkers identified in Tables 3A-3B and 4, or a sub-set thereof. In another embodiment, the kit comprises agents for use in detecting the biomarkers identified in Tables 3A-3B and 4, or a subset thereof. In related embodiments, the agents are antibodies or probes (e.g., oligonucleotides). The kit may contain about or at least about 1, 2, 3, 4, 5, 10, 50, 100, 110, 120, 130, 140, 150, 200 or more different antibodies and/or probes that each specifically recognize one of the biomarkers set forth in Tables 3A-3B and 4.

In another embodiment, the kit comprises a solid support, such as a chip, a microtiter plate or a bead or resin having capture reagents attached thereon, wherein the capture reagents bind the biomarkers of the invention. In the case of biospecfic capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for use in any of the methods described herein. In some instances, the kit comprises drug sensitivity information for chronic lymphocytic leukemias (CLLs) having different expression subtypes. The drug sensitivity data is provided in some embodiments along with instructions for selecting a patient for administration of a drug (e.g., fludarabine, ibrutinib, idelalisib, SNS-032, venetoclax, and/or navitoclax) based upon an expression subtype of a chronic lymphocytic leukemia (CLL) in the subject. In embodiments, the instructions provide suitable operational parameters in the form of a label or separate insert. For example, the instructions may inform a consumer about how to collect the sample, how to wash the probe, and/or the particular biomarkers to be detected.

In yet another embodiment, the kit can comprise one or more containers with controls (e.g., biomarker samples) to be used as standard(s) for calibration.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Dataset for Use in Creating a Comprehensive Molecular Map of CLL

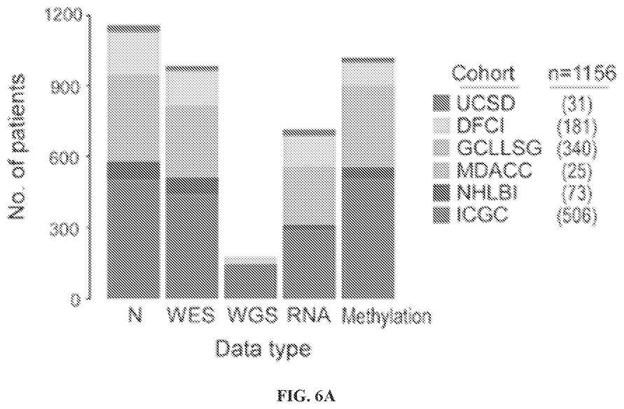

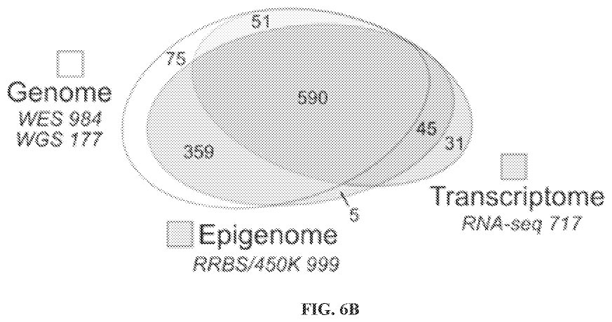

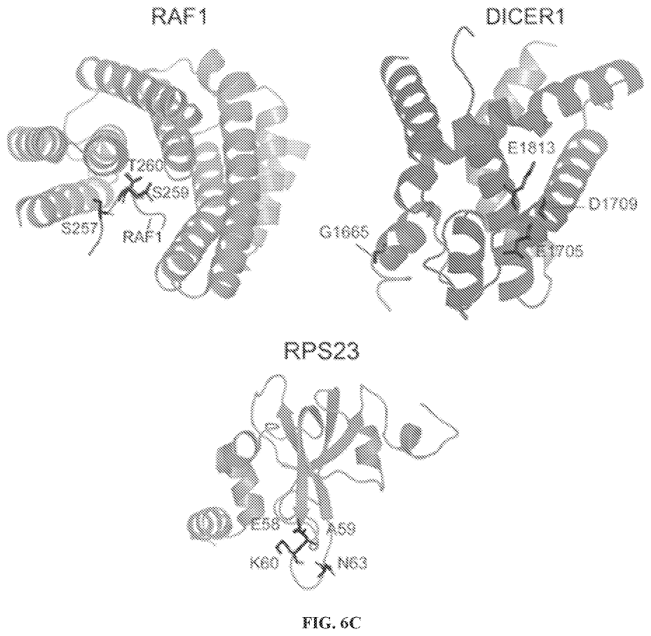

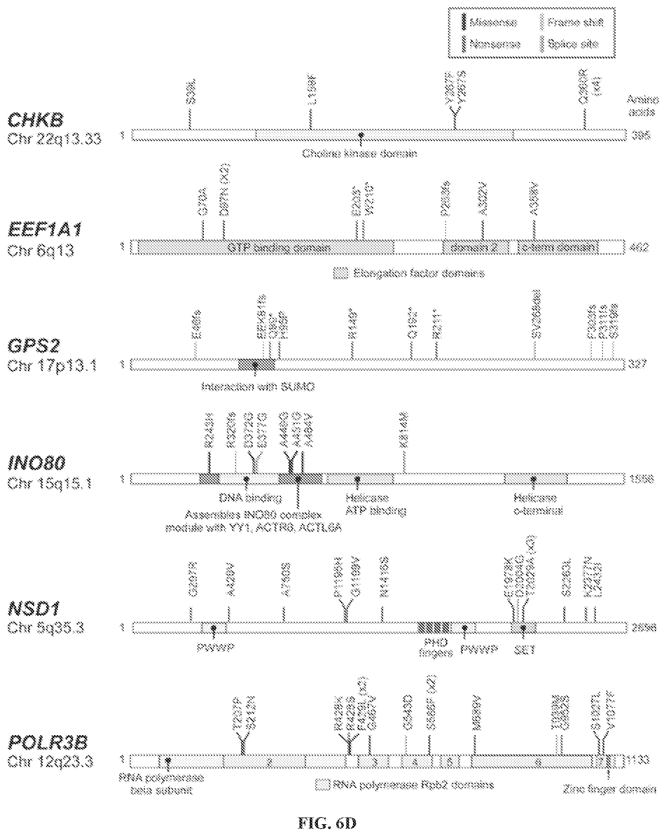

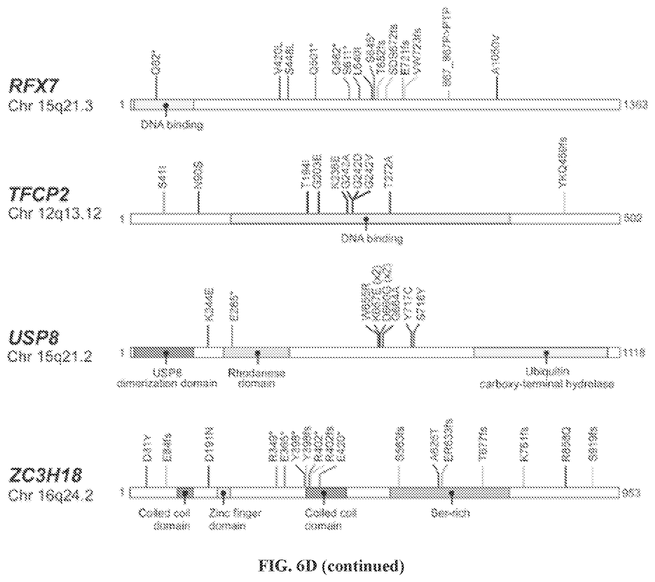

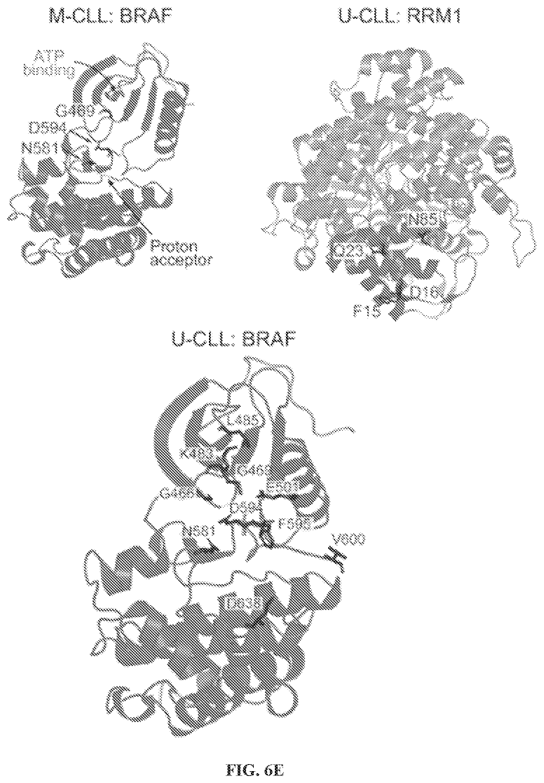

Existing and newly generated data was gathered to create a comprehensive molecular map of CLL. This encompassed samples from 1102 CLL patients and 54 patients with monoclonal B cell lymphocytosis (MBL) from which whole-exome or -genome sequencing (WES/WGS) (n=1075), RNA-sequencing (RNA-seq) (n=717) and DNA methylation data (n=999) were analyzed (FIGS. 6A and 6B). Samples were collected during active surveillance (n=687), after treatment (n=52) or at the time of enrollment in therapeutic clinical trials (n=417; n=371 treatment-naive; n=46 relapsed/refractory) (Landau, D. A. et al. Mutations driving CLL and their evolution in progression and relapse. Nature 526, 525 (2015); Puente, X. S. et al. Non-coding recurrent mutations in chronic lymphocytic leukaemia. Nature 526, 519 (2015); Gruber, M. et al. Growth dynamics in naturally progressing chronic lymphocytic leukaemia. Nature 570, 474-479 (2019); Landau, D. A. et al. The evolutionary landscape of chronic lymphocytic leukemia treated with ibrutinib targeted therapy. Nat. Commun. 8, 2185 (2017); Kasar, S. et al. Whole-genome sequencing reveals activation-induced cytidine deaminase signatures during indolent chronic lymphocytic leukaemia evolution. Nat. Commun. 6, 8866 (2015); Burger, J. A. et al. Safety and activity of ibrutinib plus rituximab for patients with high-risk chronic lymphocytic leukaemia: a single-arm, phase 2 study. Lancet Oncol. 15, 1090-1099 (2014); Burger, J. A. et al. Clonal evolution in patients with chronic lymphocytic leukaemia developing resistance to BTK inhibition. Nat. Commun. 7, 11589 (2016)). This large dataset enabled more complete delineation of the biological underpinnings of CLL and its molecular subtypes.

### Example 2: Identification of Novel CLL Drivers

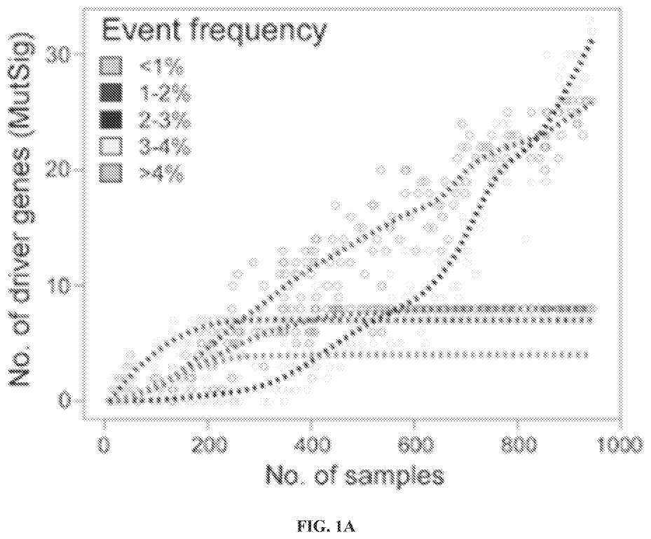

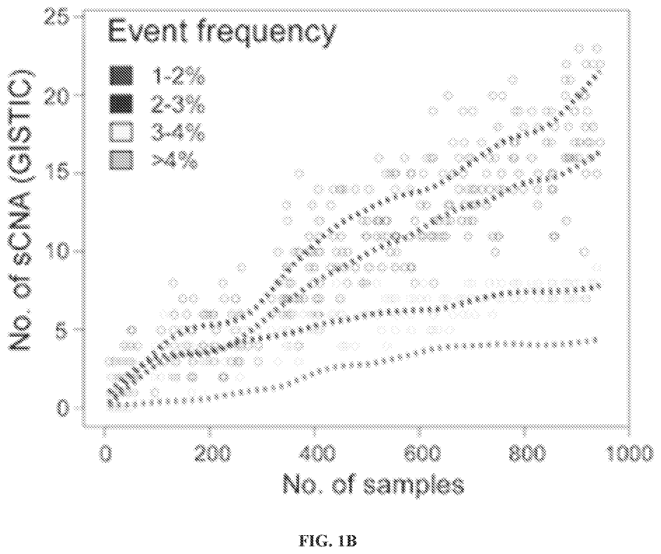

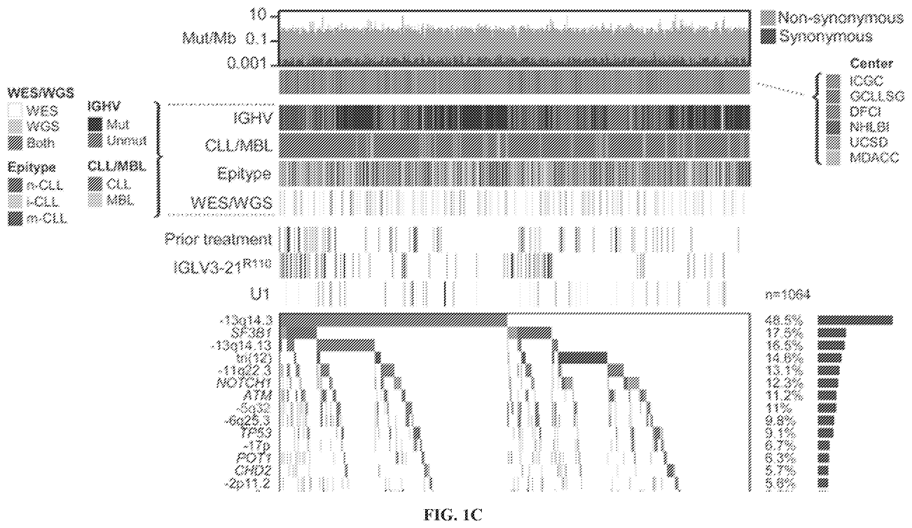

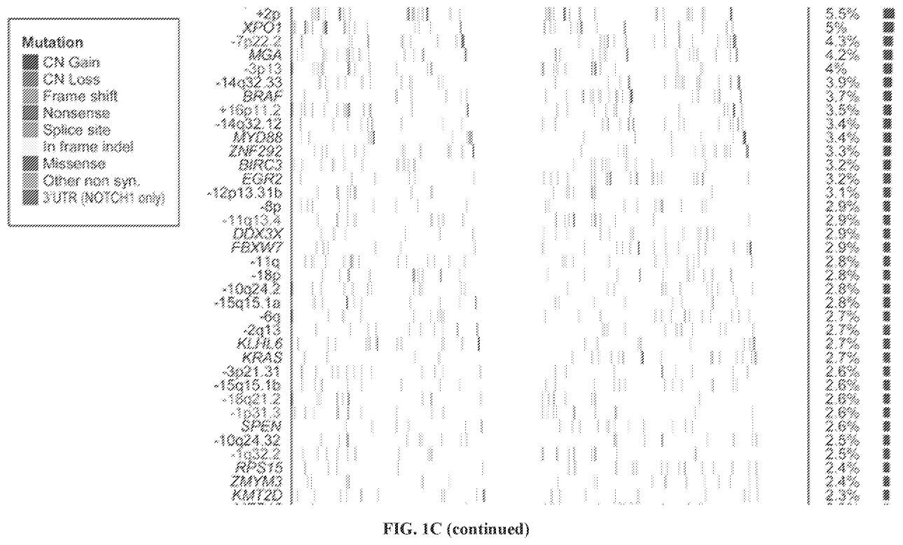

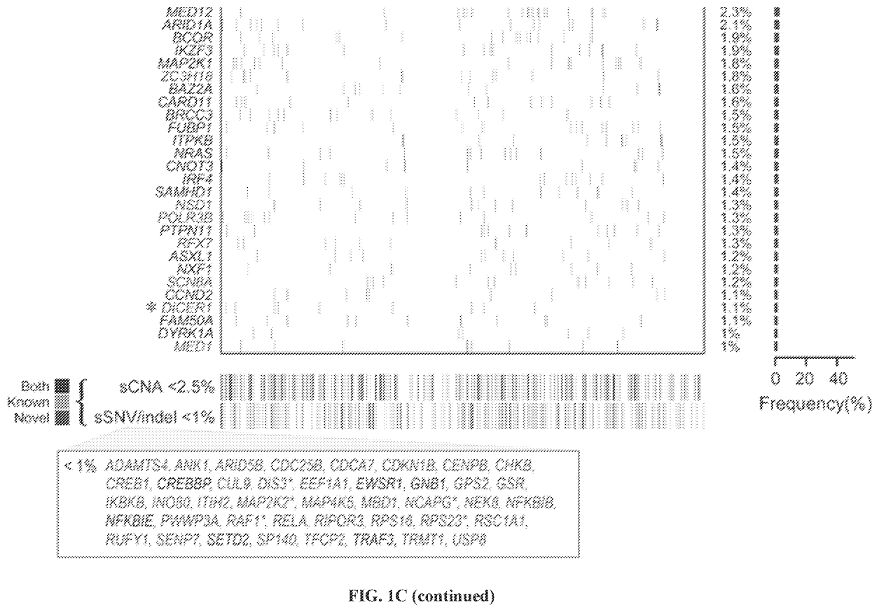

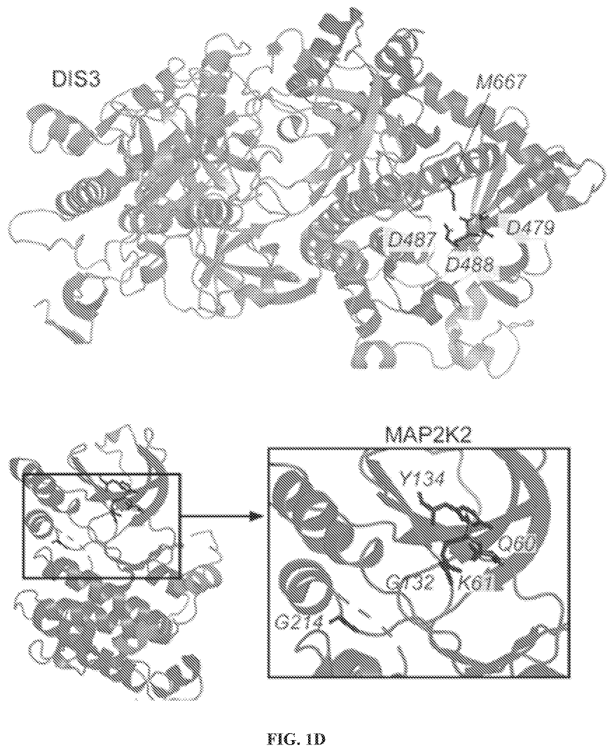

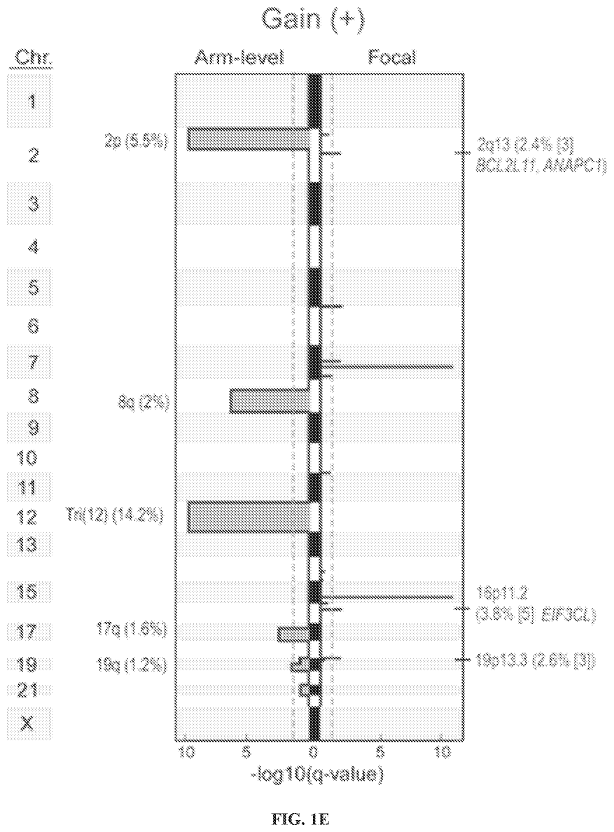

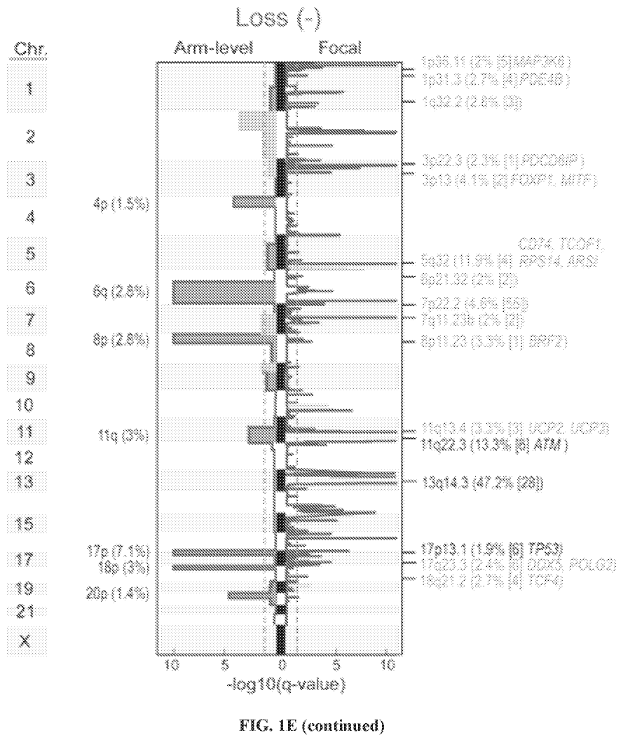

To generate a comprehensive catalogue of drivers, 984 CLL samples with whole exome sequencing (WES) were first evaluated. To ensure consistency and highest accuracy of the mutation calls, the data was reprocessed with an updated suite of tools, detecting somatic single nucleotide variants (sSNVs), short insertion/deletion mutations (indels), and copy number alterations (sCNAs). Specialized tools were also applied for detecting recently described CLL driver events such as the g.3A>C mutation of the spliceosome-related small nuclear RNA U1 (U1) and the R110 mutation in the IGLV3-21 gene (IGLV3-21). Prior power estimates suggested that with ˜1000 whole exome sequencing (WES) samples and somatic background mutation rate of ˜1/Mb in CLL, it would be feasible to discover >90% of drivers mutated in 2% of patients, whereas with ˜500 samples the power drops to 50%. To verify these estimates, a down-sampling analysis was performed and it was confirmed that the number of drivers almost doubled, increasing from an average of 38.8 with 500 cases to 74.5 with ˜1000 cases, with the majority of new drivers mutated in <2% of patients (FIG. 1A). Likewise, the increased cohort size enabled discovery of significantly recurrent somatic copy number alterations (sCNAs) across all frequencies, with the steepest increase in lower frequency drivers (FIG. 1B).

The dataset revealed 82 putative CLL driver genes based on recurrent sSNV/indel mutations (q<0.1), of which 37 were novel (FIG. 1C). Beyond the previously known CLL drivers, such as SF3B1, NOTCH1, ATM, and TP53 (mutated in 17.5%, 12.3%, 11.2%, and 9.1% of patients, respectively), as well as mutations in IGLV3-21^{R110} and U1 (mutated in 9.4% and 3.9%, respectively), the frequencies of the remaining events formed a long, gradually decreasing tail (59 of 82 drivers mutated in <2% of patients). Although most newly identified genes were mutated at low frequency, 24.1% of patients harbored at least one mutation in a novel putative driver. Notably, they were also the sole sSNV/indel driver in 4% of patients. Six additional putative driver genes were identified through spatial clustering of mutations in 3-D protein structures, using CLUMPS (Kamburov, A. et al. Comprehensive assessment of cancer missense mutation clustering in protein structures. Proc. Natl. Acad. Sci. U.S.A 112, E5486-95 (2015)), including MAP2K2, DIS3, and DICER1 (FIGS. 1D and 6C). Three MAP2K2 mutations were localized in the kinase domain, which activates ERK signaling and is functionally similar to MAP2K1, a previously identified CLL driver. DIS3 is the catalytic subunit of the RNA exosome complex involved in processing nearly all RNA species and is recurrently mutated in multiple myeloma. Two of four altered sites in DIS3 were in known cancer hotspots (D479 and D488) and located in the catalytic domain. Beyond sSNV/indels in coding regions, an analysis of 177 whole genome sequencing (WGS) did not reveal novel noncoding CLL drivers (see “Non-coding driver discovery procedure” provided below).

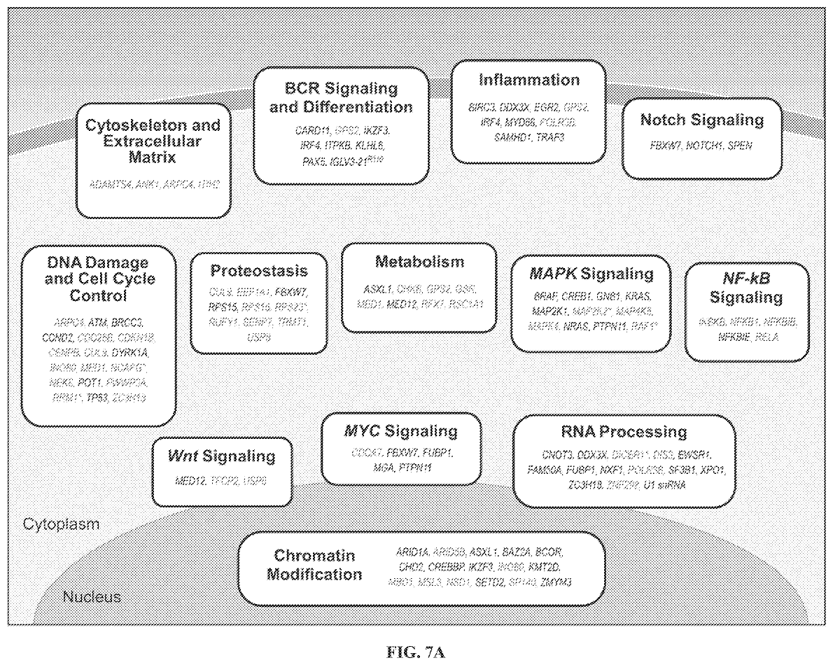

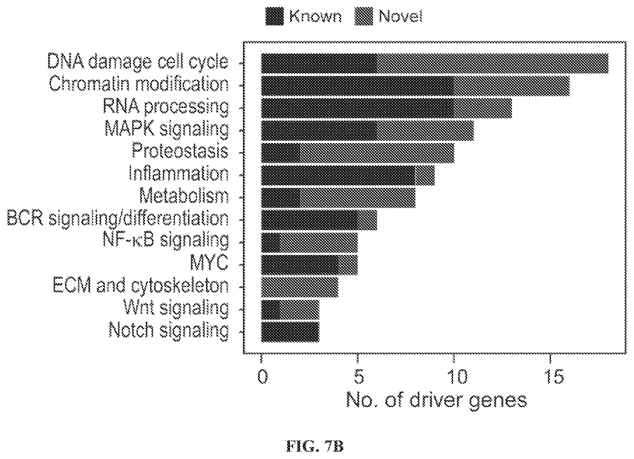

In support of the newly identified drivers, 7 (18.9%) had mutations clustered in functional domains (FIG. 6D). For example, mutations were identified in the DNA-binding domain of INO80, the catalytic subunit of a chromatin remodeling complex that regulates genome stability and is frequently mutated in hepatosplenic T cell lymphoma. Additionally, 7 (18.9%) of the novel drivers likely have a role in other mature B cell malignancies such as the tumor suppressor, RFX7, implicated in Burkitt lymphoma and diffuse large B cell lymphoma. These candidate drivers were also enriched in biological pathways known to contribute to CLL pathogenesis such as DNA damage and chromatin modification. However, they also identified processes not previously highlighted by driver genes like protein synthesis and stability as well as regulation of cytoskeletal proteins and the extracellular matrix (FIGS. 7A and 7B).

A striking new finding provided by the increased statistical power was the abundance of yet unreported focal somatic copy number alterations (sCNAs) associated with CLL, including 5 novel gains and 30 new losses (of 6 and 53 total, respectively) (FIG. 1E). A novel deletion in 5q32 (11.9% of samples) encompassed ARSI, TCOF1, CD74, and RPS14, which is part of the common deleted region in 5q-syndrome, a low-risk subtype of myelodysplastic syndrome (MDS). Two of these genes, RPS14 and TCOF1, are involved in ribosome function or biogenesis and have been implicated in inflammatory Toll-like receptor signaling in MDS models and in maintaining genomic integrity after DNA damage, respectively, suggesting that multiple genes in this region are associated with pathways involved in CLL oncogenesis. Other new deletions contain the mitochondrial uncoupling proteins UCP2 and UCP3 in 11q13.4 (3.3%) that function as tumor suppressors altering redox homeostasis and multiple other regions that include known cancer associated genes. Rarely reported arm level somatic copy number alterations (sCNAs) were also identified including 17q gain (1.6%) and 4p loss (1.5%). Altogether, these results vastly expand the map of CLL drivers and reveal convergent mechanisms through which cardinal cellular processes are altered in this disease.

### Example 3: Molecular Profiles of IGHV (Heavy Chain Variable Region of Immunoglobulin Genes) Subtypes

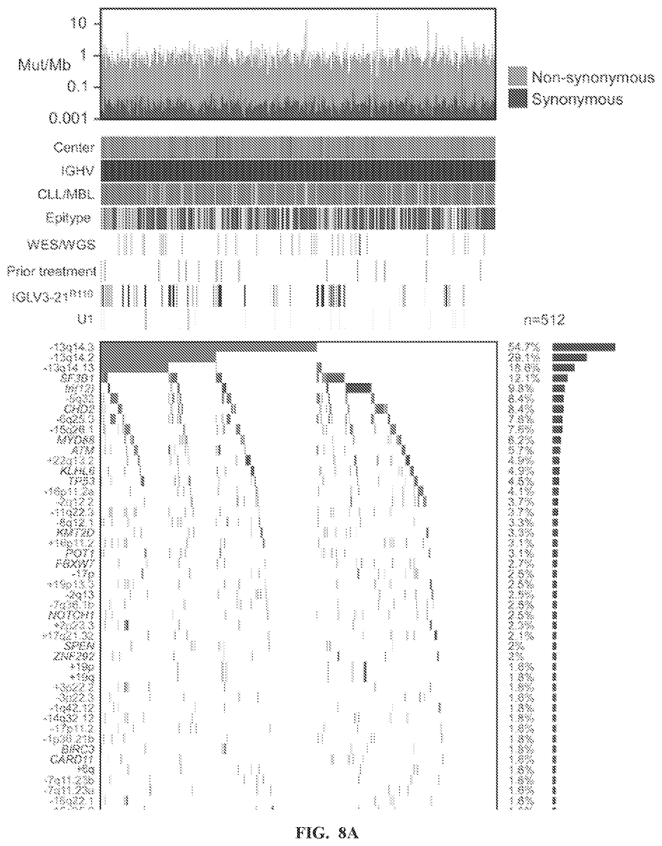

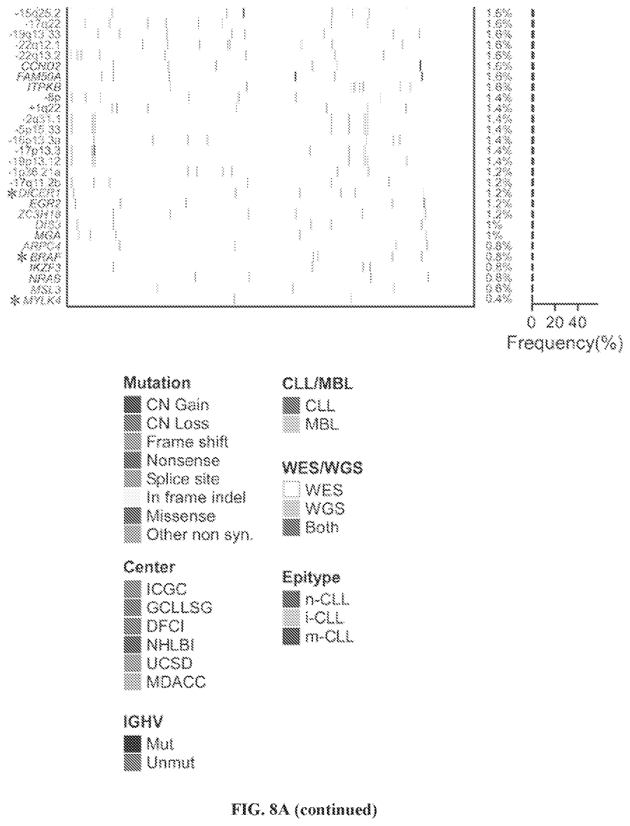

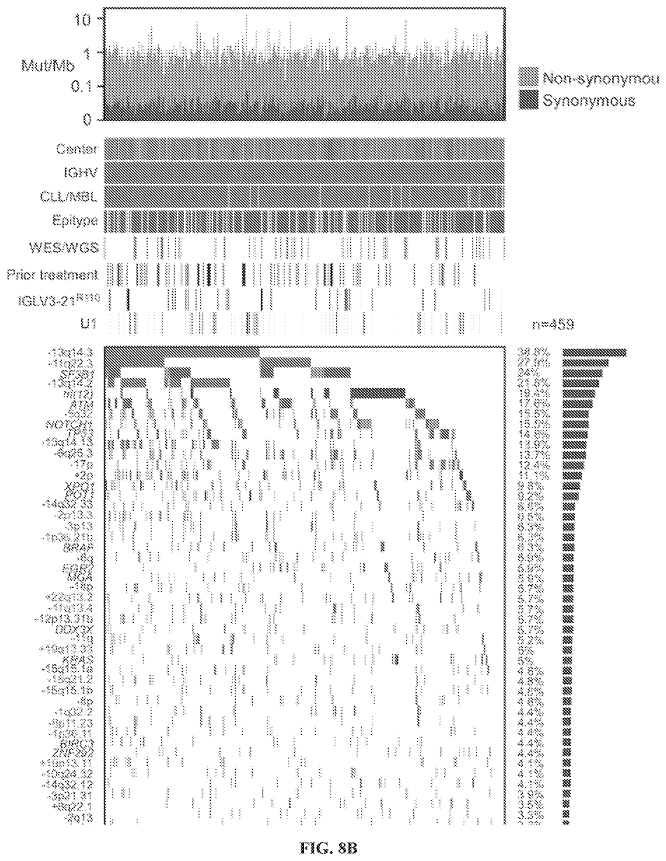

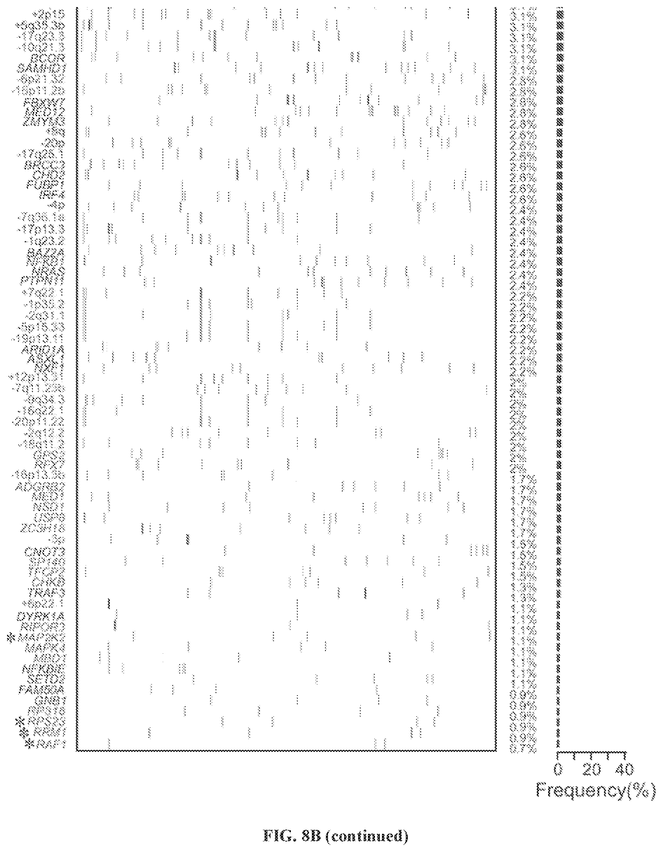

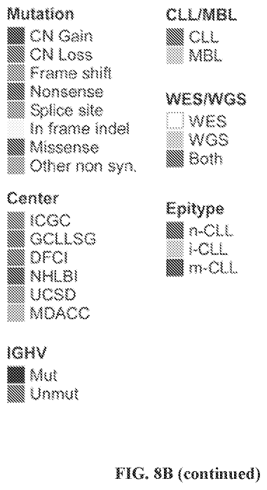

The increased cohort size was leveraged to discover distinct candidate driver genes, sCNAs, and structural variants (SVs) in 512 CLLs with mutated IGHV (heavy chain variable region of immunoglobulin genes) (M-CLLs) and 459 CLLs with unmutated IGHV (heavy chain variable region of immunoglobulin genes) (U-CLLs), greatly expanding previous work that identified only a limited number of discrete molecular characteristics associated with IGHV (heavy chain variable region of immunoglobulin genes) status. The IGHV (heavy chain variable region of immunoglobulin genes) subtype-specific mutation analyses increased sensitivity to identify 7 novel putative drivers that were not identified in the pan-CLL analysis (FIGS. 6E, 8A and 8B). In U-CLL (CLL with unmutated IGHV), this included NFKB1, a regulator of NFκB signaling, and RRM1, a catalytic subunit of ribonucleotide reductase which is critical for DNA replication and repair as well as the target of nucleoside analogs including fludarabine.

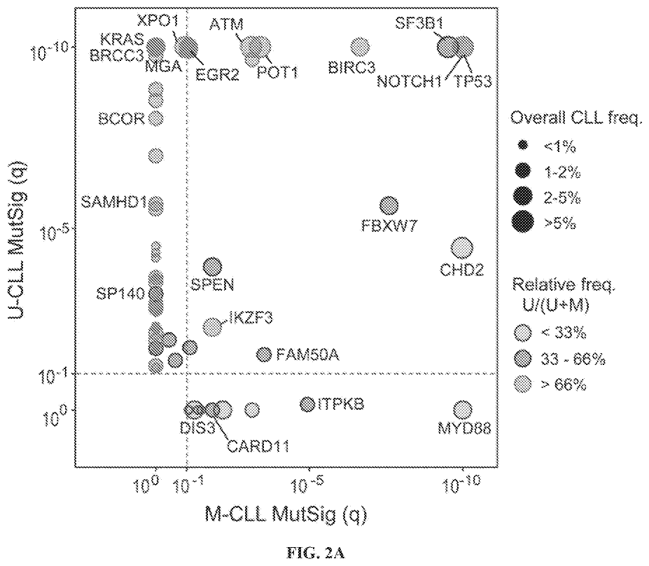

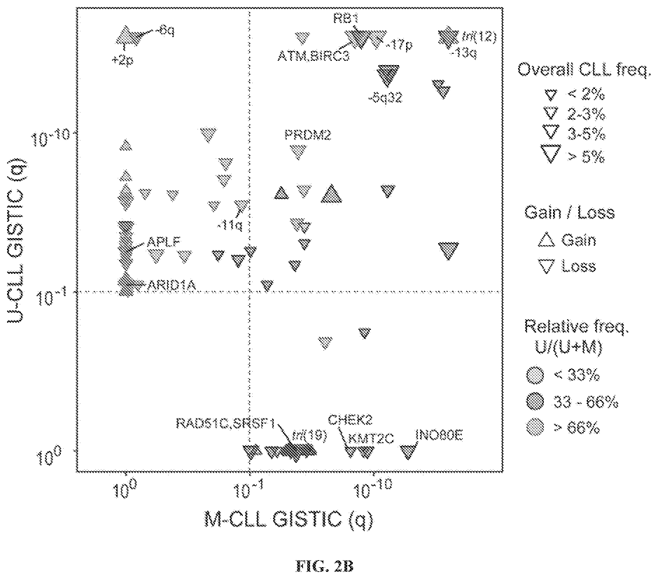

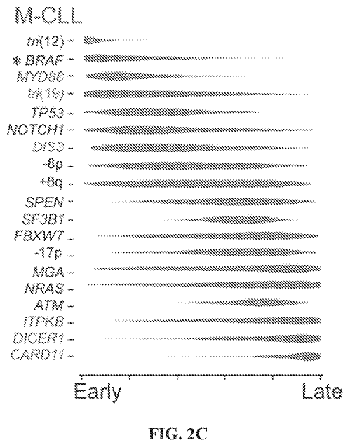

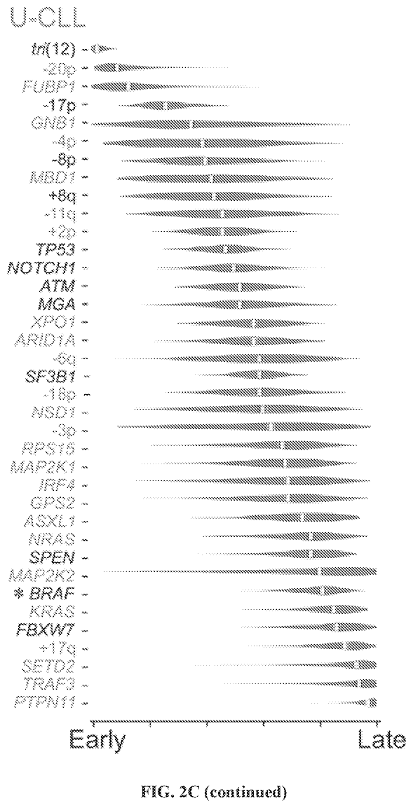

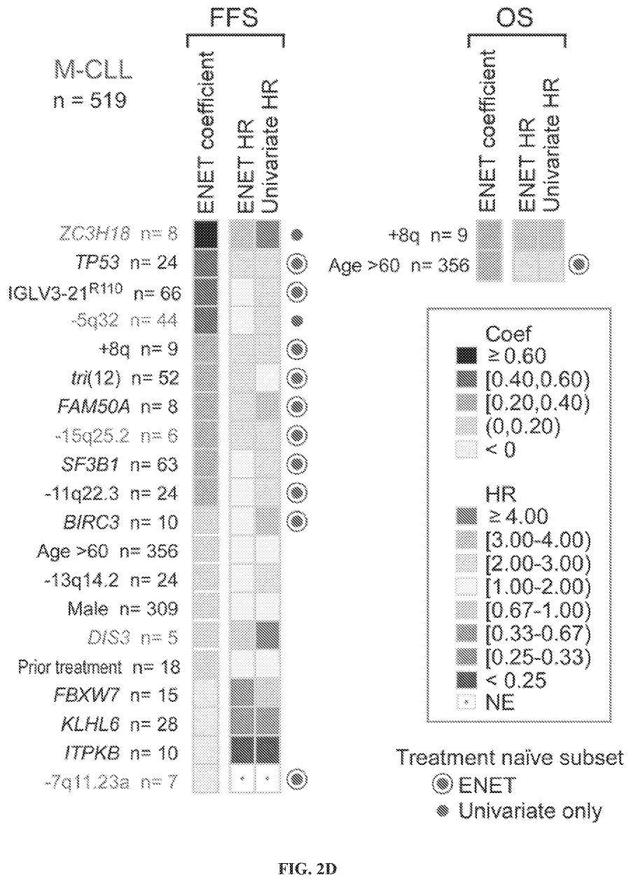

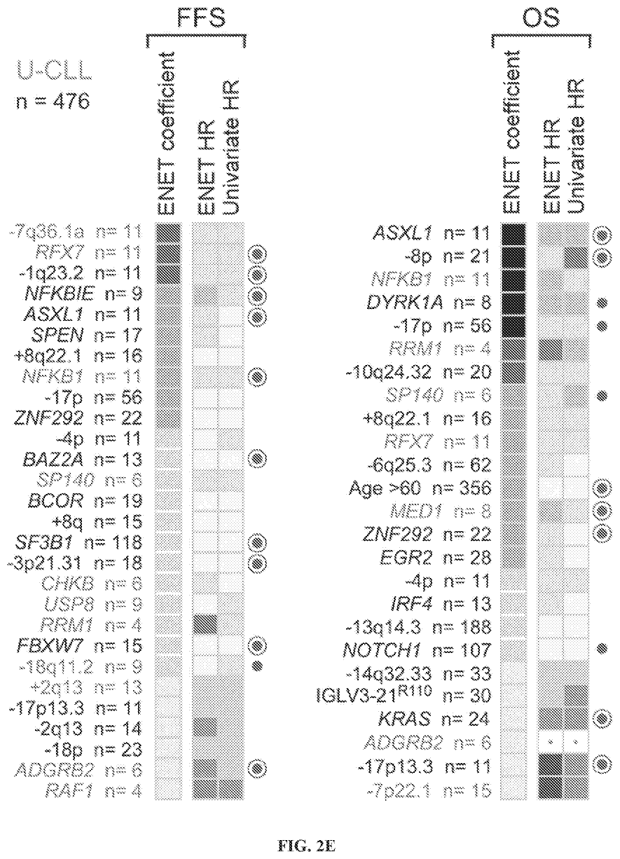

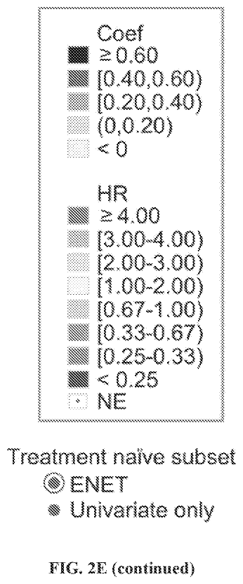

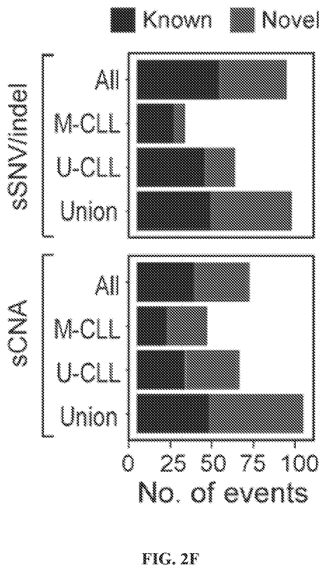

Although M-CLL (CLL with mutated IGHV) and U-CLL (CLL with unmutated IGHV) had similar cohort sizes and comparable mutational burdens in coding regions (1.14/Mb vs. 1.11/Mb medians, respectively; Wilcoxon rank-sum test p=0.979; though the mean number of clonal mutations genome-wide was increased in M-CLL (CLL with mutated IGHV)—12.6 versus 9.6, p=6×10⁻¹⁴), the number of significant putative drivers was much higher in U-CLL (CLL with unmutated IGHV) (54 versus 25 genes, respectively; ratio 2.16, Binomial test p=0.0015). To ensure that this difference was not due to prior therapy, a comparison was made between only treatment-naive samples and each cohort (n=375; M-CLL (CLL with mutated IGHV) was downsampled), and again more drivers were found in U-CLL (CLL with unmutated IGHV) (ratio 2.82, one sample t-test p=5×10⁻¹¹). Most drivers were significant in either M-CLL (CLL with mutated IGHV) (n=9) or U-CLL (CLL with unmutated IGHV) (n=38) while only a minority were significant in both subgroups (n=16, 25.4% of total) (FIG. 2A). Of these shared drivers, 10 of the 16 were twice as frequent in U-CLL, consistent with the increased driver frequency in this subtype.

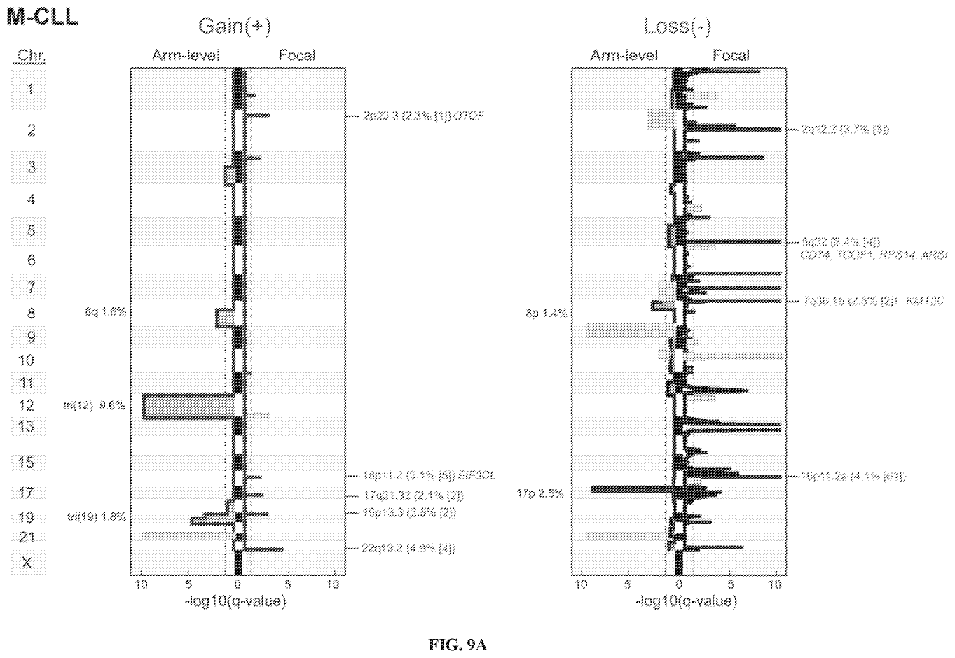

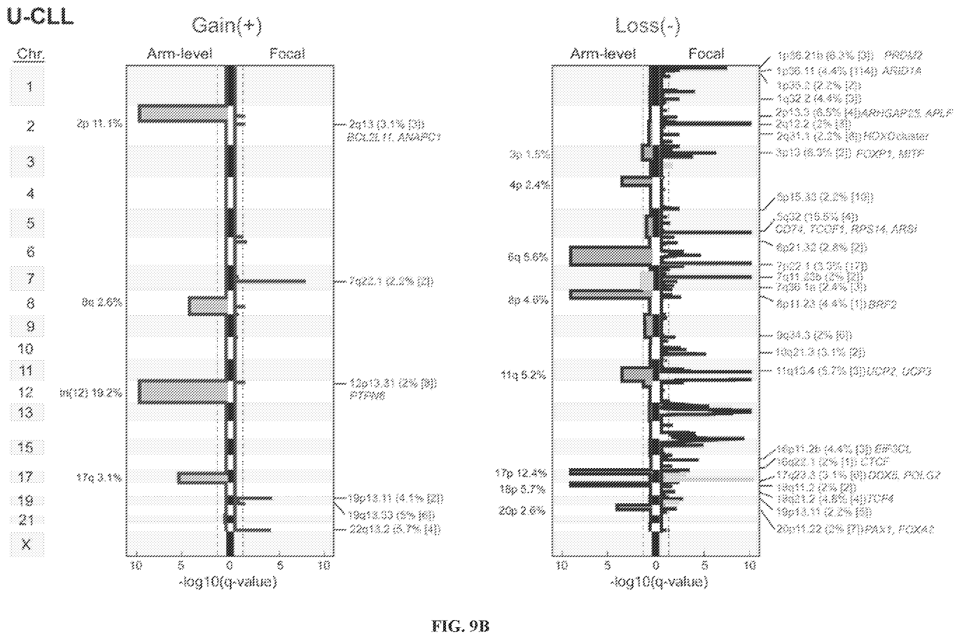

IGHV subtypes were also distinguished by somatic copy number alteration (sCNA) profiles (70 of 90 in either M-CLL (CLL with mutated IGHV) or U-CLL (CLL with unmutated IGHV) vs. 20 of 90 shared) (FIGS. 2B, 9A and 9B). Trisomy 19 (1.8%) was only observed in M-CLL, consistent with previous studies. In contrast, 8 arm level events including 2p gain (11.1%) and loss of 6q (5.6%) were only significant in U-CLL. The majority of focal events distinguishing the IGHV (heavy chain variable region of immunoglobulin genes) subtypes were novel, comprising 18 of 23 events enriched in M-CLL (CLL with mutated IGHV) and 25 of 37 in U-CLL, and some provided orthogonal evidence for the CLL driver genes discovered through mutation analysis. For example, loss of 1p36.11 (4.4%) contained ARID1A, a known driver gene, and both this somatic copy number alteration (sCNA) and somatic single nucleotide variant (sSNV) were only significant in U-CLL. The somatic copy number alterations (sCNAs) identified in each subtype also emphasized underlying biology important in CLL leukemogenesis. In M-CLL, the region in 7q36.1b loss (2.5%) included KMT2C, a lysine-specific methyltransferase involved in epigenetic regulation. A related tumor suppressor, KMT2D, is likely a candidate driver also enriched specifically in M-CLL.

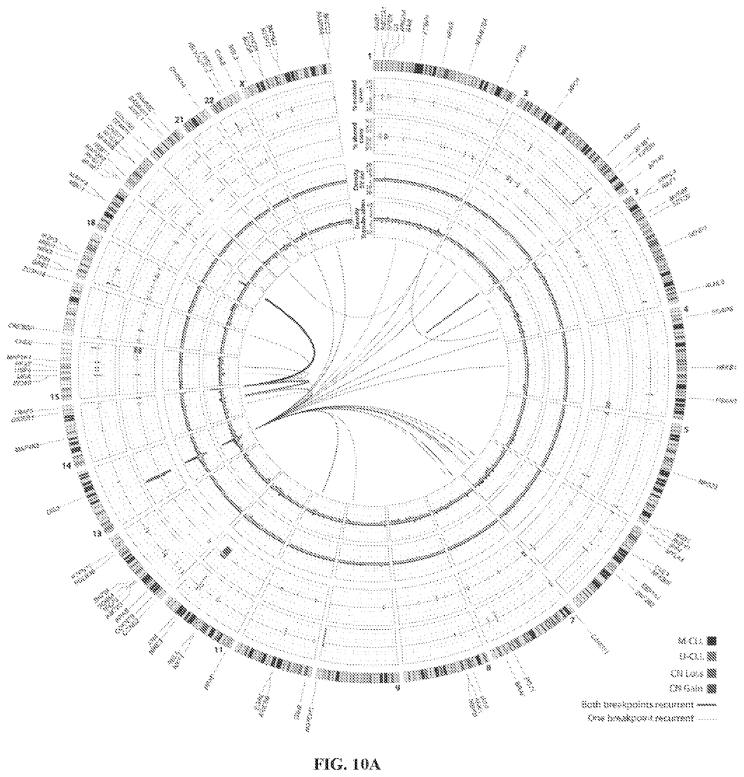

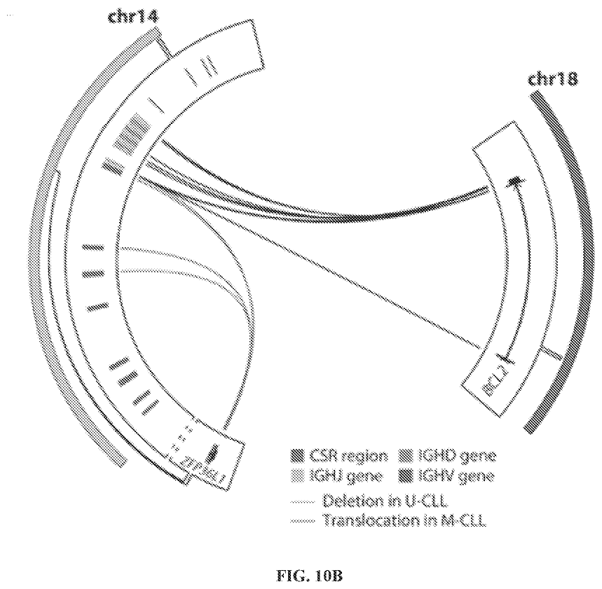

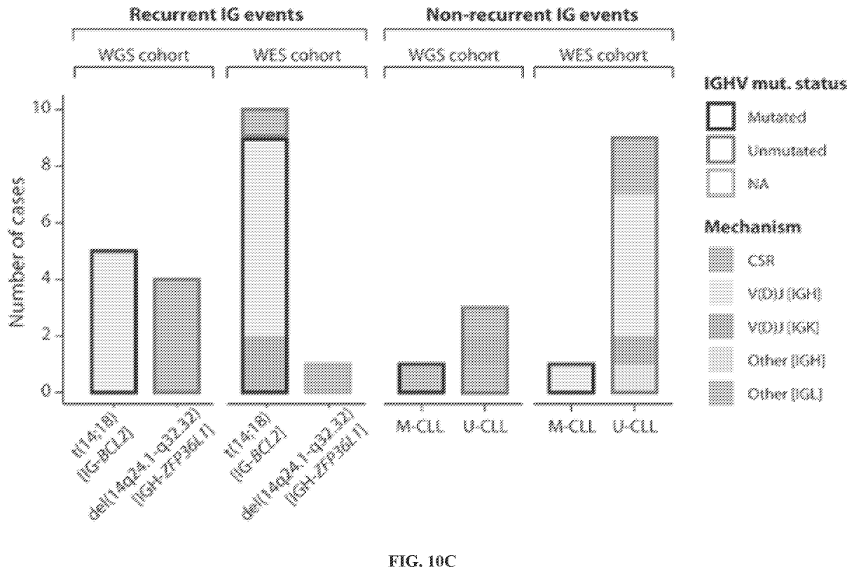

Differences were further identified between M-CLL (CLL with mutated IGHV) and U-CLL (CLL with unmutated IGHV) on the basis of SVs. From 177 whole genome sequencing (WGS) (88 M-CLL, 87 U-CLL (CLL with unmutated IGHV) and 2 non-evaluable), 681 SV breakpoints were discovered in 141 (79.7%) patients (average of 4.8 per patient). The most recurrent SVs involving the immunoglobulin (Ig) loci (as identified by IgCaller (Nadeu, F. et al. IgCaller for reconstructing immunoglobulin gene rearrangements and oncogenic translocations from whole-genome sequencing in lymphoid neoplasms. Nat. Commun. 11, 3390 (2020))) distinguished M-CLL (CLL with mutated IGHV) from U-CLL (CLL with unmutated IGHV) (FIGS. 10A-10B). It was confirmed that the most common Ig translocation partner in M-CLL (CLL with mutated IGHV) was BCL2 (5 of 88 cases, 5.7%). Conversely, a large 37-Mb deletion in chromosome 14 was identified in U-CLL (CLL with unmutated IGHV) (4 of 87 cases, 4.6%), which deletes candidate CLL drivers (DICER1, TRAF3) and directly perturbs ZFP36L1, a tumor suppressor that down-regulates NOTCH1. The rearrangement mechanism also differed between these events, with aberrant V(D)J recombination driving the BCL2 events in M-CLL (CLL with mutated IGHV) and class-switch recombination facilitating the ZFP36L1-associated deletions in U-CLL (CLL with unmutated IGHV) (FIG. 10B). These different patterns and underlying mechanisms were confirmed in the whole exome sequencing (WES) cohort where IgCaller detected 9 of 10 additional cases with BCL2 translocations in M-CLL (CLL with mutated IGHV) (FIG. 10C).

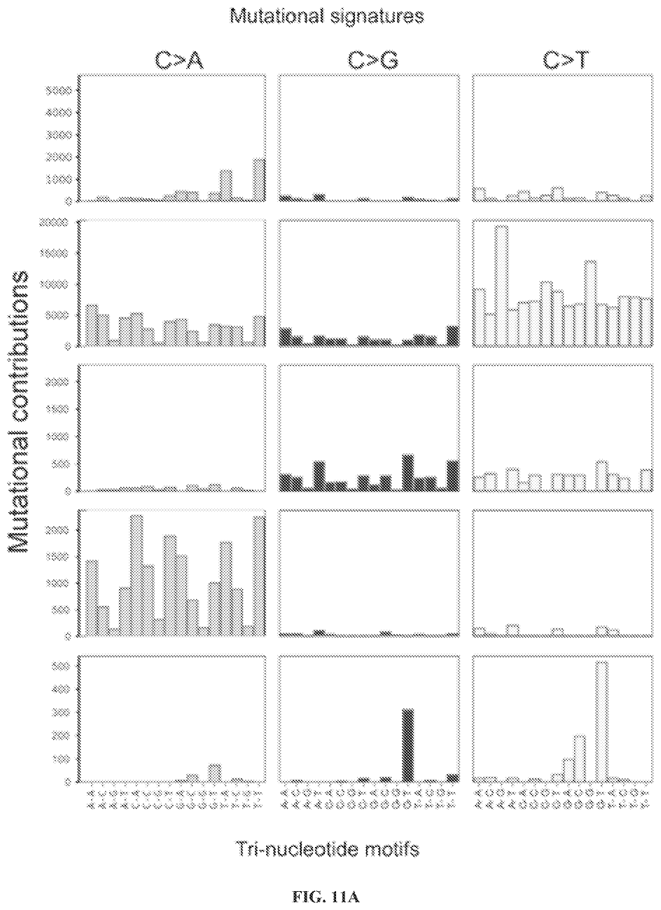

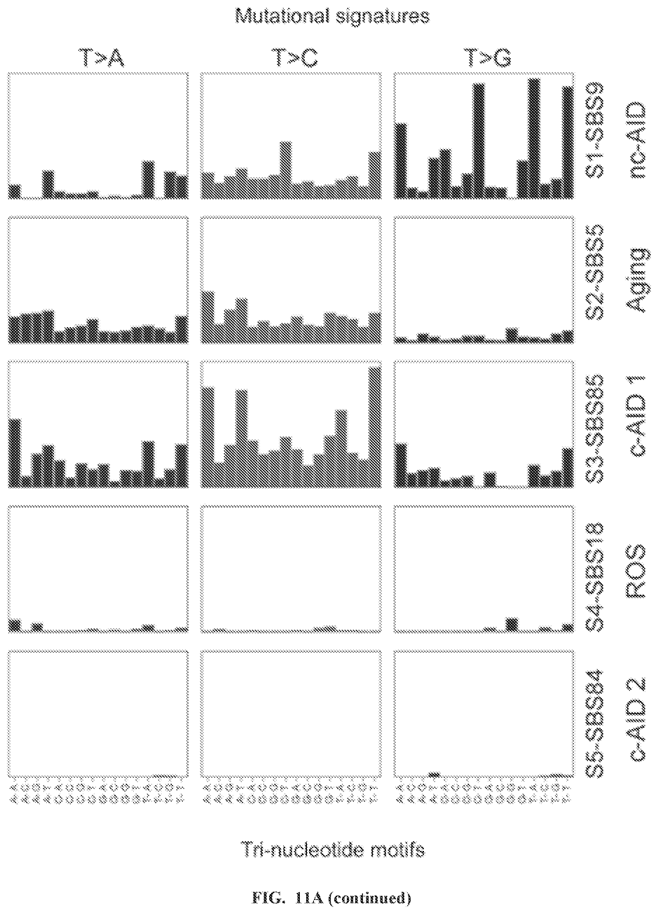

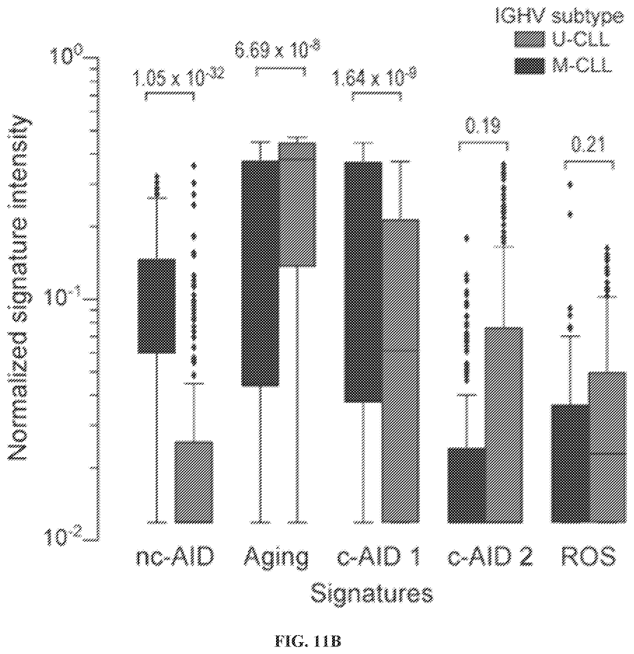

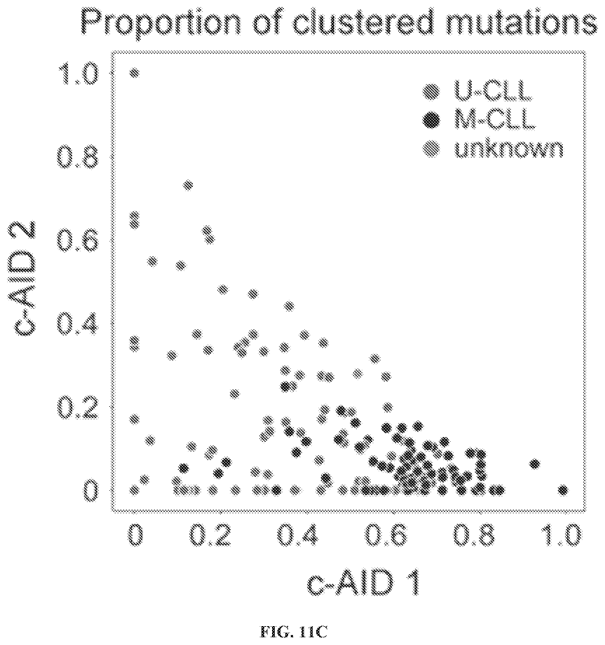

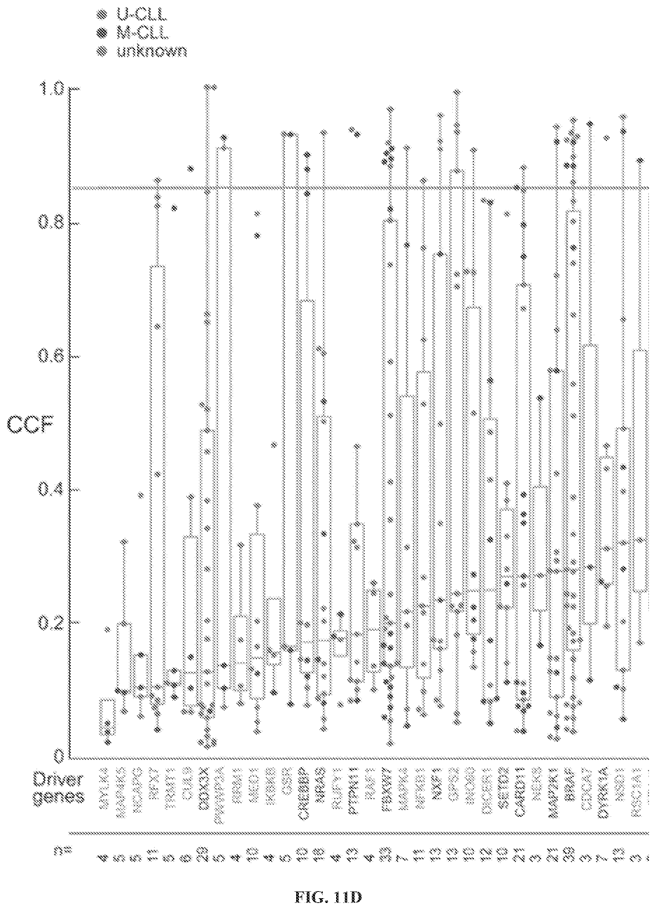

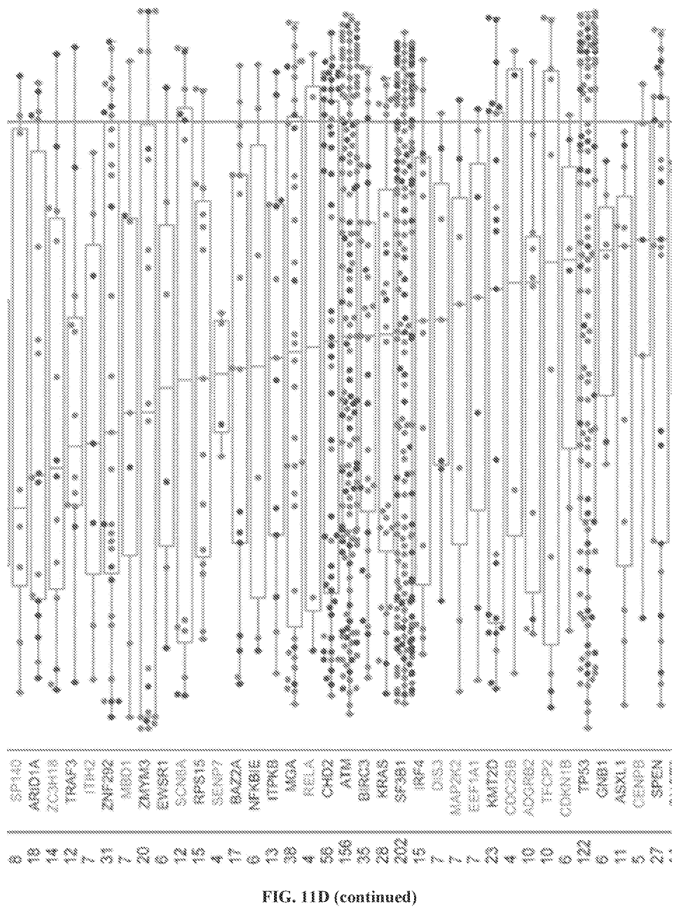

To evaluate possible differences in mechanisms of somatic mutation generation active in M-CLL (CLL with mutated IGHV) and U-CLL, mutation signature analysis was performed on 177 whole genome sequencing (WGS) and identified activity of 5 mutational processes (FIG. 11A). In addition to confirming the presence of the aging, canonical activation-induced cytidine deaminase (c-AID) and non-canonical AID (nc-AID) related signatures, evidence of signature SBS18 was also found, likely due to damage from reactive oxygen species, and splitting of the c-AID signatures (SBS84 and SBS85). Of note, clustered mutations in U-CLL (CLL with unmutated IGHV) were enriched in SBS84 (Wilcoxon rank-sum test, p=0.193), whereas SBS85 was more prevalent in M-CLL, likely reflecting unique mutational processes arising from AID in each subtype (p=1.64×10⁻⁹, FIGS. 11B and 11C, see also “Mutational signatures review” provided below).

Further highlighting the differences between M-CLL (CLL with mutated IGHV) and U-CLL, inferred timing of acquired sSNV/indels and arm level somatic copy number alterations (sCNAs) were detected when analyzed by PhylogicNDT (Leshchiner, I. et al. Comprehensive analysis of tumour initiation, spatial and temporal progression under multiple lines of treatment. bioRxiv 508127 (2019)) (FIG. 2C). Trisomy 12 was an early event and shared drivers such as TP53 and NOTCH1 were intermediate in both CLL subtypes. In contrast, acquisition of BRAF mutations was an early event in M-CLL (CLL with mutated IGHV) but occurred late in U-CLL (CLL with unmutated IGHV) (q<0.1). Of those drivers specifically enriched per subtype, MYD88 was an early event in M-CLL (CLL with mutated IGHV) whereas 20p loss and FUBP1 alterations may be initiating lesions in U-CLL. The temporal acquisition of sSNV/indels was separately assessed by analyzing their cancer cell fractions (CCF) (FIG. 11D). Only 12 (12.4%) of the driver genes had predominantly clonal events with a median CCF>85%, and 6 of these 12 driver genes were novel including MSL3 and USP8 identified in M-CLL (CLL with mutated IGHV) and U-CLL, respectively. This panoply of genetic differences underscores M-CLL (CLL with mutated IGHV) and U-CLL (CLL with unmutated IGHV) as distinct molecular entities and support their unique trajectories of leukemogenesis.

Given these differences, the clinical impact of putative genetic drivers found in each IGHV (heavy chain variable region of immunoglobulin genes) subtype was analyzed (FIGS. 2D and 2E, Tables 1A-1E and 2A-2E). Eighteen novel candidate drivers were associated with either failure-free survival (FFS) and/or overall survival (OS) (5 of 17 in M-CLL (CLL with mutated IGHV) and 13 of 41 in U-CLL). In M-CLL, ZC3H18 and loss of 5q32 and 15q25.2 were novel alterations associated with risk of short failure free survival (FFS) in addition to known factors such as TP53 and IGLV3-21^{R110} mutations. The prognostic impact of many of these novel putative drivers was also supported when the dataset was restricted to only treatment-naive, non-trial samples (n=394) (Tables 1A-1E and 2A-2E). Only two features were associated with reduced survival in M-CLL, which were age >60 years and gain of 8q, the chromosomal arm containing MYC. Relative to M-CLL, U-CLL (CLL with unmutated IGHV) had more genetic changes associated with outcome (41 versus 17 in M-CLL, Binomial test p=0.002). RFX7 and NFKB1 were novel candidate driver genes associated with poor failure free survival (FFS) and OS, although only failure free survival (FFS) was shorter in the treatment-naive subset (n=247, Tables 1A-1E and 2A-2E). The prognostic impact of known but less frequent drivers, such as NFKBIE and ASXL1, was also evident in addition to verifying the known effects of more common features such as 17p deletion. Not being bound by theory, 17p deletion and TP53 mutations significantly co-occur, which partially explains why only one was significant the modeling. Further analysis of either alteration alone or in combination demonstrated that TP53 mutation in the absence of 17p deletion was not associated with adverse outcomes in U-CLL. This likely reflects the use of contemporary therapies such as ibrutinib and venetoclax where TP53 mutation alone has not been shown to influence prognosis.

In summary, aggregation of three separate genomic analyses of the entire cohort (n=984), M-CLL (CLL with mutated IGHV) (n=512), and U-CLL (CLL with unmutated IGHV) (n=459) revealed a total of 97 putative CLL driver genes and 105 somatic copy number alterations (sCNAs) in addition to U1 and IGLV3-21R″° mutations (FIG. 2F). Previous studies demonstrated that ˜10% of patients lacked an identifiable driver. In the current analysis considering all known and candidate drivers, the percent of patients lacking at least one potential driver was reduced to 3.8%. These patients without identifiable drivers were predominantly M-CLL (CLL with mutated IGHV) (Fisher's Exact test p=1.04×10⁻⁷; 6.6% relative to 0.6% in U-CLL), confirming yet another distinction between IGHV (heavy chain variable region of immunoglobulin genes) subtypes.

Tables 1A-1E and 2A-2E relate to the impact of genetic alterations in M-CLL (CLL with mutated IGHV) and U-CLL (CLL with unmutated IGHV) on clinical outcomes.

TABLE 1A
Overall survival analysis in M-CLL (CLL with mutated IGHV) (N = 516)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| Age >=60 | 356 | 69 | 2.93 | 1.68 | 5.1 | 0.0001 | 2.984 | 0.341 |
| gain_8q | 9 | 1.7 | 3.58 | 1.45 | 8.83 | 0.0057 | 3.861 | 0.22 |
| loss_8p | 9 | 1.7 | 2.97 | 1.09 | 8.09 | 0.0338 | — | — |
| loss_17q11.2b | 6 | 1.2 | 4.28 | 1.35 | 13.56 | 0.0134 | — | — |
| ATM | 27 | 5.2 | 2.16 | 1.04 | 4.47 | 0.0386 | — | — |
| BIRC3 | 10 | 1.9 | 3.03 | 1.11 | 8.28 | 0.0304 | — | — |
| KMT2D | 18 | 3.5 | 2.59 | 1.2 | 5.61 | 0.0159 | — | — |
| TP53 | 24 | 4.6 | 2.64 | 1.28 | 5.45 | 0.0089 | — | — |
| Male | 309 | 60 | 1.65 | 1.07 | 2.55 | 0.0237 | — | — |
| prior | 18 | 3.5 | 2.31 | 1.07 | 5.01 | 0.0337 | — | — |
| treatment | | | | | | | | |

TABLE 1B
Failure free survival analysis in M-CLL (CLL with mutated IGHV) (N = 516)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| ZC3H18 | 8 | 1.5 | 4.37 | 2.05 | 9.33 | 0.0001 | 3.293 | 0.68723 |
| TP53 | 24 | 4.6 | 2.47 | 1.49 | 4.105 | 0.0005 | 2.112 | 0.50301 |
| IGLV321_R110 | 66 | 13 | 2.552 | 1.86 | 3.506 | <.0001 | 1.481 | 0.44423 |
| loss_5q32 | 44 | 8.5 | 2.507 | 1.73 | 3.64 | <.0001 | 1.736 | 0.4186 |
| gain_8q | 9 | 1.7 | 2.678 | 1.26 | 5.691 | 0.0104 | 2.512 | 0.38973 |
| tri_12 | 52 | 10 | 1.717 | 1.19 | 2.481 | 0.004 | 2.094 | 0.38002 |
| FAM50A | 8 | 1.5 | 3.837 | 1.8 | 8.172 | 0.0005 | 2.251 | 0.35716 |
| loss_15q25.2 | 6 | 1.2 | 2.134 | 0.88 | 5.174 | 0.0935 | 2.423 | 0.32816 |
| SF3B1 | 63 | 12 | 2.415 | 1.76 | 3.322 | <.0001 | 1.491 | 0.31916 |
| loss_11q22.3 | 24 | 4.6 | 2.041 | 1.28 | 3.263 | 0.0029 | 1.854 | 0.31181 |
| BIRC3 | 10 | 1.9 | 3.283 | 1.68 | 6.428 | 0.0005 | 1.111 | 0.13345 |
| Age >=60 | 356 | 69 | 1.25 | 0.95 | 1.66 | 0.117 | 1.415 | 0.1059 |
| loss_13q14.2 | 158 | 30 | 1.346 | 1.03 | 1.758 | 0.029 | 1.326 | 0.07661 |
| Male | 309 | 60 | 1.44 | 1.1 | 1.88 | 0.0074 | 1.182 | 0.07627 |
| DIS3 | 5 | 1 | 4.045 | 1.66 | 9.853 | 0.0021 | 0.892 | 0.01577 |
| prior treatment | 18 | 3.5 | 1.884 | 1.08 | 3.297 | 0.0264 | 1.318 | 0.00601 |
| FBXW7 | 15 | 2.9 | 0.525 | 0.2 | 1.412 | 0.2019 | 0.489 | −0.02906 |
| KLHL6 | 28 | 5.4 | 0.482 | 0.23 | 1.023 | 0.0574 | 0.468 | −0.16415 |
| ITPKB | 10 | 1.9 | 0.179 | 0.03 | 1.272 | 0.0855 | 0.232 | −0.20129 |
| loss_7q11.23a | 7 | 1.4 | 0 | 0 | 0 | 0.9681 | 0 | −0.36321 |
| loss_17p | 13 | 2.5 | 2.094 | 1.03 | 4.241 | 0.0401 | — | — |
| loss_6q25.3 | 39 | 7.5 | 2.168 | 1.45 | 3.249 | 0.0002 | — | — |
| loss_8q12.1 | 18 | 3.5 | 2.431 | 1.42 | 4.176 | 0.0013 | — | — |
| ATM | 27 | 5.2 | 1.965 | 1.23 | 3.145 | 0.0049 | — | — |
| SPEN | 9 | 1.7 | 2.188 | 1.03 | 4.643 | 0.0414 | — | — |

TABLE 1C
Overall survival analysis in U-CLL (CLL with unmutated IGHV) (N = 476)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| ASXL1 | 11 | 2.3 | 3.729 | 1.96 | 7.092 | <.0001 | 3.672 | 0.8583 |
| loss_8p | 21 | 4.4 | 4.32 | 2.57 | 7.276 | <.0001 | 2.586 | 0.8441 |
| NFKB1 | 11 | 2.3 | 2.98 | 1.52 | 5.83 | 0.0014 | 3.322 | 0.8274 |
| DYRK1A | 8 | 1.7 | 3.414 | 1.51 | 7.734 | 0.0033 | 3.365 | 0.6704 |
| loss_17p | 56 | 12 | 2.583 | 1.76 | 3.786 | <.0001 | 2.342 | 0.6094 |
| RRM1 | 4 | 0.8 | 3.163 | 1.01 | 9.931 | 0.0485 | 6.22 | 0.5696 |
| loss_10q24.32 | 20 | 4.2 | 2.378 | 1.29 | 4.379 | 0.0054 | 2.664 | 0.4192 |
| SP140 | 6 | 1.3 | 3.024 | 1.24 | 7.369 | 0.0149 | 2.065 | 0.393 |
| gain_8q22.1 | 16 | 3.4 | 2.2 | 1.12 | 4.308 | 0.0215 | 2.672 | 0.3657 |
| RFX7 | 11 | 2.3 | 2.02 | 0.95 | 4.304 | 0.0683 | 2.393 | 0.3317 |
| loss_6q25.3 | 62 | 13 | 1.765 | 1.18 | 2.639 | 0.0057 | 2.023 | 0.3267 |
| Age >=60 | 356 | 69 | 1.678 | 1.24 | 2.264 | 0.0007 | 1.681 | 0.3029 |
| MED1 | 8 | 1.7 | 2.397 | 0.98 | 5.86 | 0.0553 | 3.962 | 0.2768 |
| ZNF292 | 22 | 4.6 | 1.616 | 0.9 | 2.908 | 0.1094 | 2.129 | 0.2696 |
| EGR2 | 28 | 5.9 | 1.618 | 0.94 | 2.794 | 0.084 | 2.194 | 0.2561 |
| loss_4p | 11 | 2.3 | 2.541 | 1.25 | 5.172 | 0.0101 | 2.388 | 0.1632 |
| IRF4 | 13 | 2.7 | 1.692 | 0.75 | 3.821 | 0.206 | 2.413 | 0.0797 |
| loss_13q14.3 | 188 | 40 | 1.289 | 0.97 | 1.72 | 0.0852 | 1.313 | 0.0712 |
| NOTCH1 | 107 | 22 | 1.324 | 0.96 | 1.835 | 0.0913 | 1.238 | 0.0174 |
| loss_14q32.33 | 33 | 6.9 | 0.523 | 0.25 | 1.113 | 0.0927 | 0.561 | −0.0314 |
| IGLV321_R110 | 30 | 6.3 | 0.418 | 0.19 | 0.944 | 0.0358 | 0.552 | −0.1609 |
| KRAS | 24 | 5 | 0.337 | 0.13 | 0.912 | 0.0323 | 0.345 | −0.382 |
| ADGRB2 | 6 | 1.3 | 0 | 0 | 0 | 0.9756 | 0 | −0.4824 |
| loss_17p13.3 | 11 | 2.3 | 0.354 | 0.11 | 1.115 | 0.076 | 0.137 | −0.5902 |
| loss_7p22.1 | 15 | 3.2 | 0.353 | 0.11 | 1.104 | 0.0734 | 0.125 | −0.6483 |
| BAZ2A | 13 | 2.7 | 2.161 | 1.1 | 4.227 | 0.0244 | — | — |
| prior treatment | 18 | 3.5 | 1.884 | 1.08 | 3.297 | 0.0264 | — | — |
| Male | 309 | 60 | 1.44 | 1.1 | 1.88 | 0.0074 | — | — |
| loss_3p | 6 | 1.3 | 2.797 | 1.04 | 7.555 | 0.0425 | — | — |
| gain_17q | 14 | 2.9 | 2.069 | 1.09 | 3.919 | 0.0256 | — | — |
| TP53 | 69 | 15 | 1.893 | 1.31 | 2.747 | 0.0008 | — | — |

TABLE 1D
Failure free survival analysis in U-CLL (CLL with unmutated
IGHV) failure free survival (FFS) (N = 476)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| loss_7q36.1a | 11 | 2.3 | 2.845 | 1.55 | 5.209 | 7.00E−04 | 2.645 | 0.5161 |
| RFX7 | 11 | 2.3 | 2.385 | 1.31 | 4.35 | 0.0046 | 2.743 | 0.4733 |
| loss_1q23.2 | 11 | 2.3 | 2.52 | 1.38 | 4.615 | 0.0027 | 2.322 | 0.4052 |
| NFKBIE | 9 | 1.9 | 2.24 | 1.11 | 4.519 | 0.0243 | 3.461 | 0.3634 |
| ASXL1 | 11 | 2.3 | 1.977 | 1.08 | 3.605 | 0.0261 | 2.516 | 0.3421 |
| SPEN | 17 | 3.6 | 1.787 | 1.07 | 2.999 | 0.0279 | 2.356 | 0.2771 |
| gain_8q22.1 | 16 | 3.4 | 1.94 | 1.16 | 3.258 | 0.0123 | 1.838 | 0.269 |
| NFKB1 | 11 | 2.3 | 2.049 | 1.09 | 3.844 | 0.0255 | 2.153 | 0.2436 |
| loss_17p | 56 | 12 | 1.527 | 1.14 | 2.038 | 0.0041 | 1.428 | 0.242 |
| ZNF292 | 22 | 4.6 | 1.604 | 1.03 | 2.493 | 0.036 | 1.95 | 0.24 |
| loss_4p | 11 | 2.3 | 2.001 | 1.1 | 3.654 | 0.0239 | 1.476 | 0.1495 |
| BAZ2A | 13 | 2.7 | 1.788 | 1.01 | 3.18 | 0.048 | 1.975 | 0.1441 |
| SP140 | 6 | 1.3 | 2.261 | 1.01 | 5.074 | 0.0478 | 2.582 | 0.1173 |
| BCOR | 19 | 4 | 1.473 | 0.9 | 2.403 | 0.1206 | 1.871 | 0.1173 |
| gain_8q | 15 | 3.2 | 1.644 | 0.98 | 2.756 | 0.0595 | 1.856 | 0.0778 |
| SF3B1 | 118 | 25 | 1.272 | 1.01 | 1.596 | 0.0377 | 1.147 | 0.0673 |
| loss_3p21.31 | 18 | 3.8 | 1.669 | 1.01 | 2.756 | 0.0453 | 1.474 | 0.0615 |
| CHKB | 6 | 1.3 | 1.929 | 0.86 | 4.329 | 0.1111 | 2.51 | 0.0547 |
| USP8 | 9 | 1.9 | 2.138 | 1.06 | 4.315 | 0.034 | 1.566 | 0.0426 |
| RRM1 | 4 | 0.8 | 2.176 | 0.81 | 5.84 | 0.1229 | 4.041 | 0.0361 |
| FBXW7 | 15 | 3.2 | 1.502 | 0.86 | 2.612 | 0.1501 | 1.775 | 0.0125 |
| loss_18q11.2 | 9 | 1.9 | 2.586 | 1.33 | 5.029 | 0.0051 | 1.202 | 0.0065 |
| gain_2q13 | 13 | 2.7 | 0.619 | 0.33 | 1.163 | 0.1362 | 0.917 | −0.0069 |
| loss_17p13.3 | 11 | 2.3 | 0.781 | 0.42 | 1.466 | 0.4417 | 0.623 | −0.0244 |
| loss_2q13 | 14 | 2.9 | 0.683 | 0.36 | 1.283 | 0.236 | 0.425 | −0.0709 |
| loss_18p | 23 | 4.8 | 0.741 | 0.48 | 1.152 | 0.1828 | 0.699 | −0.1028 |
| ADGRB2 | 6 | 1.3 | 0.505 | 0.19 | 1.353 | 0.1742 | 0.436 | −0.1217 |
| RAF1 | 4 | 0.8 | 0.332 | 0.08 | 1.329 | 0.1191 | 0.263 | −0.4374 |
| gain_7q22.1 | 10 | 2.1 | 1.93 | 1.03 | 3.623 | 0.0406 | — | — |
| gain_12p13.31 | 9 | 1.9 | 2.424 | 1.25 | 4.713 | 0.0091 | — | — |
| loss_1p35.2 | 10 | 2.1 | 2.344 | 1.21 | 4.557 | 0.012 | — | — |
| loss_2q31.1 | 11 | 2.3 | 1.937 | 1.06 | 3.539 | 0.0315 | — | — |
| loss_5p15.33 | 10 | 2.1 | 2.142 | 1.14 | 4.027 | 0.018 | — | — |
| loss_17q25.1 | 13 | 2.7 | 1.868 | 1.05 | 3.325 | 0.0337 | — | — |
| loss_19p13.11 | 11 | 2.3 | 2.158 | 1.18 | 3.945 | 0.0125 | — | — |
| loss_20p11.22 | 9 | 1.9 | 2.452 | 1.26 | 4.773 | 0.0083 | — | — |

TABLE 1E
Legend for Tables 1A-1D

| Column | Description |
|---|---|
| Variable | Feature tested in modeling |
| N | Number of patients with feature |
| % | Percent patients with feature |
| Univariate | Hazard Ratio from univariate Cox regression model |
| Hazard Ratio | |
| Univariate | Hazard Ratio Lower Confidence Limit from univariate |
| Lower | Cox regression model |
| Confidence | |
| Limit | |
| Univariate | Hazard Ratio Upper Confidence Limit from univariate |
| Upper | Cox regression model |
| Confidence | |
| Limit | |
| Univariate p- | p-value from univariate Cox regression model |
| value | |
| ENET Hazard | Hazard Ratio in multivariable model (computed by a |
| Ratio | Cox regression multivariable model for all variables |
| | with non-zero coefficient in the elastic net (ENET) |
| | model) |
| ENET | Elastic net coefficient ( — indicates 0 coefficient and |
| Coefficient | exclusion from the model) |

TABLE 2A
Overall survival analysis in treatment-naïve M-CLL (CLL with mutated
IGHV) (N = 394) (excluding patients enrolled on therapeutic clinical trial)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| ATM | 7 | 1.78 | 5.01 | 2.0 | 12.48 | 0.0005 | 5.89 | 1.130774 |
| KMT2D | 13 | 3.3 | 4.81 | 2.19 | 10.56 | <.0001 | 5.39 | 1.090552 |
| BIRC3 | 6 | 1.52 | 4.13 | 1.29 | 13.19 | 0.0166 | 6.32 | 0.619063 |
| Age >=60 | 276 | 70.05 | 3.62 | 1.79 | 7.34 | 0.0004 | 2.96 | 0.551015 |
| vs. <60 | | | | | | | | |
| sex_M | 175 | 44.4 | 0.5 | 0.3 | 0.84 | 0.008 | 0.41 | 0.307066 |
| loss_15q25.2 | 6 | 1.52 | 2.56 | 0.8 | 8.19 | 0.112 | 2.5 | 0.077613 |
| gain_2p23.3 | 9 | 2.28 | 0 | 0 | 0 | 0.9828 | 0 | −0.15961 |
| TP53 | 18 | 4.57 | 2.51 | 1.08 | 5.82 | 0.0321 | — | — |

TABLE 2B
Failure free survival analysis in treatment-naïve M-CLL (CLL with mutated
IGHV) (N = 394) (excluding patients enrolled on therapeutic clinical trial)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| IGLV321_R110 | 24 | 6.09 | 4.36 | 2.76 | 6.87 | <.0001 | 4.77 | 1.008003 |
| FAM50A | 5 | 1.27 | 7.31 | 2.97 | 17.98 | <.0001 | 4.41 | 0.699985 |
| tri_12 | 34 | 8.63 | 2.22 | 1.45 | 3.39 | 0.0002 | 2.37 | 0.530398 |
| SF3B1 | 33 | 8.38 | 2.87 | 1.92 | 4.31 | <.0001 | 1.85 | 0.452906 |
| loss_15q25.2 | 6 | 1.52 | 3.02 | 1.24 | 7.35 | 0.015 | 4.08 | 0.437509 |
| loss_7q36.1b | 12 | 3.05 | 2.44 | 1.24 | 4.78 | 0.0095 | 2.89 | 0.395916 |
| loss_11q22.3 | 11 | 2.79 | 3.18 | 1.63 | 6.23 | 0.0007 | 3.08 | 0.175489 |
| BIRC3 | 6 | 1.52 | 3.73 | 1.64 | 8.48 | 0.0017 | 2.15 | 0.166525 |
| TP53 | 18 | 4.57 | 2.08 | 1.16 | 3.74 | 0.0142 | 1.55 | 0.147472 |
| gain_8q | 6 | 1.52 | 2.42 | 0.99 | 5.89 | 0.0521 | 1.76 | 0.006663 |
| loss_7q11.23a | 6 | 1.52 | 0 | 0 | 0 | 0.9706 | 0 | −0.10722 |
| POT1 | 5 | 1.27 | 1.56 | 0.58 | 4.21 | 0.3778 | 0.17 | −0.14535 |
| loss_5q32 | 6 | 1.52 | 2.32 | 1.03 | 5.25 | 0.0428 | — | — |
| loss_6q25.3 | 5 | 1.27 | 2.53 | 1.04 | 6.17 | 0.0408 | — | — |
| ATM | 7 | 1.78 | 3.39 | 1.59 | 7.23 | 0.0016 | — | — |
| ZC3H18 | 6 | 1.52 | 4.61 | 1.88 | 11.31 | 0.0009 | — | — |

TABLE 2C
Overall survival analysis in treatment-naïve U-CLL (CLL with unmutated
IGHV) (N = 247) (excluding patients enrolled on therapeutic clinical trial)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| loss_8p | 8 | 3.24 | 5.39 | 2.44 | 11.87 | <.0001 | 4.08 | 0.756319 |
| ASXL1 | 7 | 2.83 | 5.95 | 2.55 | 13.88 | <.0001 | 2.7 | 0.545447 |
| MED1 | 4 | 1.62 | 3.75 | 1.35 | 10.38 | 0.0111 | 20.52 | 0.306873 |
| Age >=60 | 138 | 55.87 | 1.85 | 1.201 | 2.82 | 0.005 | 1.74 | 0.163065 |
| vs. <60 | | | | | | | | |
| ZNF292 | 17 | 6.88 | 2.12 | 1.09 | 4.11 | 0.0266 | 2.99 | 0.053089 |
| loss_17p13.3 | 7 | 2.83 | 0.33 | 0.08 | 1.33 | 0.1187 | 0.06 | −0.1063 |
| KRAS | 12 | 4.86 | 0.15 | 0.02 | 1.08 | 0.0601 | 0.12 | −0.14182 |
| loss_5p15.33 | 6 | 2.43 | 0 | 0 | 0 | 0.9803 | 0 | −0.18219 |
| DYRK1A | 4 | 1.62 | 5.06 | 1.58 | 16.17 | 0.0063 | — | — |
| SP140 | 4 | 1.62 | 3.23 | 1.02 | 10.27 | 0.0472 | — | — |
| loss_3p | 2 | 0.81 | 5.54 | 1.35 | 22.74 | 0.0175 | — | — |
| loss_17p | 21 | 8.5 | 2.04 | 1.05 | 3.98 | 0.0366 | — | — |
| loss_10q21.3 | 1 | 0.4 | 12.77 | 1.71 | 95.68 | 0.0132 | — | — |
| BIRC3 | 11 | 4.45 | 2.4 | 1.1 | 5.21 | 0.0276 | — | — |
| NOTCH1 | 62 | 25.1 | 1.63 | 1.05 | 2.531 | 0.0294 | — | — |

TABLE 2D
Failure free survival analysis in treatment-naïve** U-CLL (CLL with unmutated
IGHV) (N = 247) (excluding patients enrolled on therapeutic clinical trial)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| loss_16p13.3b | 3 | 1.21 | 24.01 | 7 | 82.32 | <.0001 | 24.66 | 2.263446 |
| ARID1A | 4 | 1.62 | 3.95 | 1.45 | 10.76 | 0.0073 | 6.05 | 0.770894 |
| BIRC3 | 11 | 4.45 | 3 | 1.61 | 5.56 | 0.0005 | 2.85 | 0.707225 |
| loss_1q23.2 | 7 | 2.83 | 2.91 | 1.36 | 6.25 | 0.0061 | 3.13 | 0.608947 |
| NFKB1 | 5 | 2.02 | 3.3 | 1.34 | 8.15 | 0.0096 | 2.47 | 0.588284 |
| RFX7 | 6 | 2.43 | 2.47 | 1.09 | 5.59 | 0.0304 | 3.41 | 0.386357 |
| loss_{——}18q21.2 | 4 | 1.62 | 4.56 | 1.67 | 12.46 | 0.0031 | 2.54 | 0.374099 |
| EGR2 | 11 | 4.45 | 2.07 | 1.13 | 3.82 | 0.0193 | 2.08 | 0.372246 |
| BRAF | 18 | 7.29 | 1.65 | 0.97 | 2.8 | 0.0639 | 2.34 | 0.337521 |
| NFKBIE | 6 | 2.43 | 3.31 | 1.45 | 7.55 | 0.0044 | 1.59 | 0.335621 |
| NXF1 | 6 | 2.43 | 2.29 | 1.01 | 5.17 | 0.0471 | 3.1 | 0.301088 |
| loss_11q22.3 | 63 | 25.51 | 1.6 | 1.18 | 2.18 | 0.0027 | 1.54 | 0.296803 |
| PTPN11 | 6 | 2.43 | 2.06 | 0.91 | 4.66 | 0.0829 | 2.73 | 0.208305 |
| SF3B1 | 47 | 19.03 | 1.4 | 1 | 1.96 | 0.0499 | 1.67 | 0.20066 |
| BAZ2A | 5 | 2.02 | 2.48 | 1.01 | 6.07 | 0.0473 | 1.89 | 0.189892 |
| loss_5q32 | 6 | 2.43 | 2.23 | 0.98 | 5.07 | 0.055 | 1.96 | 0.151661 |
| FBXW7 | 10 | 4.05 | 1.58 | 0.83 | 2.99 | 0.1603 | 1.61 | 0.075601 |
| tri_12 | 56 | 22.67 | 1.32 | 0.96 | 1.8 | 0.0856 | 1.24 | 0.07342 |
| loss_2q13 | 10 | 4.05 | 1.61 | 0.82 | 3.14 | 0.1652 | 1.9 | 0.06959 |
| ASXL1 | 7 | 2.83 | 1.84 | 0.86 | 3.92 | 0.1162 | 1.83 | 0.067098 |
| loss_3p21.31 | 6 | 2.43 | 1.6 | 0.71 | 3.61 | 0.2597 | 2.13 | 0.044029 |
| DYRK1A | 4 | 1.62 | 2.79 | 1.03 | 7.56 | 0.0433 | 1.97 | 0.032988 |
| NOTCH1 | 62 | 25.1 | 1.22 | 0.89 | 1.66 | 0.2165 | 1.24 | 0.007316 |
| loss_8p | 8 | 3.24 | 1.66 | 0.82 | 3.38 | 0.1593 | 1.29 | 0.006541 |
| ADGRB2 | 2 | 0.81 | 0.52 | 0.13 | 2.11 | 0.3628 | 0.69 | −0.02203 |
| BRCC3 | 7 | 2.83 | 0.76 | 0.34 | 1.7 | 0.4993 | 0.62 | −0.02293 |
| MAP2K2 | 3 | 1.21 | 0.24 | 0.03 | 1.71 | 0.1538 | 0.25 | −0.48625 |
| MAP2K1 | 6 | 2.43 | 0.25 | 0.06 | 1.01 | 0.0523 | 0.16 | −0.74324 |
| RPS23 | 1 | 0.4 | 8.53 | 1.16 | 62.79 | 0.0353 | — | — |
| loss_8p11.23 | 5 | 2.02 | 2.53 | 1.04 | 6.17 | 0.0412 | — | — |
| loss_18q11.2 | 7 | 2.83 | 3.23 | 1.5 | 6.93 | 0.0027 | — | — |
| MED1 | 4 | 1.62 | 2.75 | 1.01 | 7.44 | 0.0471 | — | — |
| NFKB1 | 5 | 2.02 | 3.3 | 1.34 | 8.15 | 0.0096 | — | — |

TABLE 2E
Legend for Tables 2A-2D

| Column | Description |
|---|---|
| Variable | Feature tested in modeling |
| N | Number of patients with feature |
| % | Percent patients with feature |
| Univariate | Hazard Ratio from univariate Cox regression model |
| Hazard Ratio | |
| Univariate | Hazard Ratio Lower Confidence Limit from univariate |
| Lower | Cox regression model |
| Confidence | |
| Limit | |
| Univariate | Hazard Ratio Upper Confidence Limit from univariate |
| Upper | Cox regression model |
| Confidence | |
| Limit | |
| Univariate p- | p-value from univariate Cox regression model |
| value | |
| ENET Hazard | Hazard Ratio in multivariable model (computed by a |
| Ratio | Cox regression multivariable model for all variables |
| | with non-zero coefficient in the elastic net (ENET) |
| | model; see Methods) |
| ENET | Elastic net coefficient ( — indicates 0 coefficient and |
| Coefficient | exclusion from the model) |

### Example 4: CLL Subtypes Based on Epigenetic and Transcriptomic Features

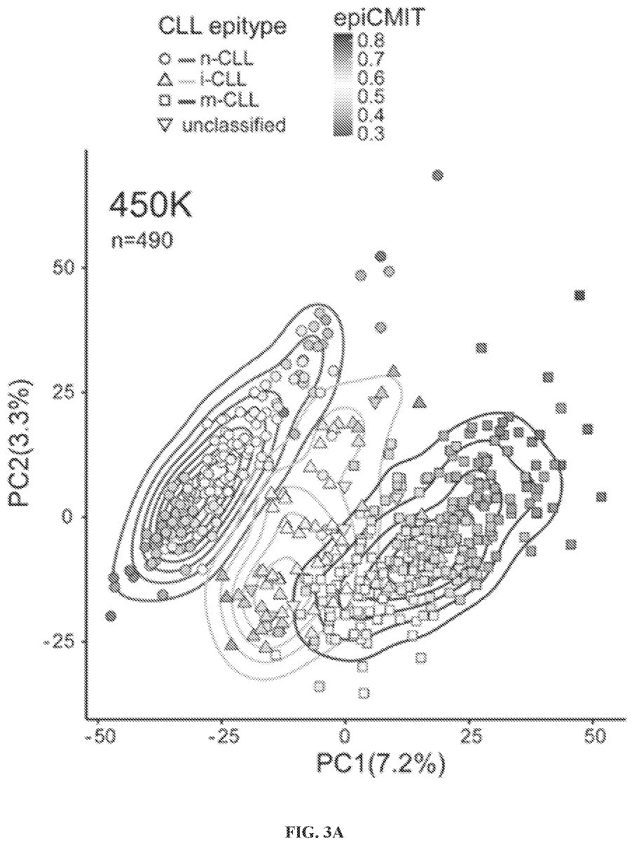

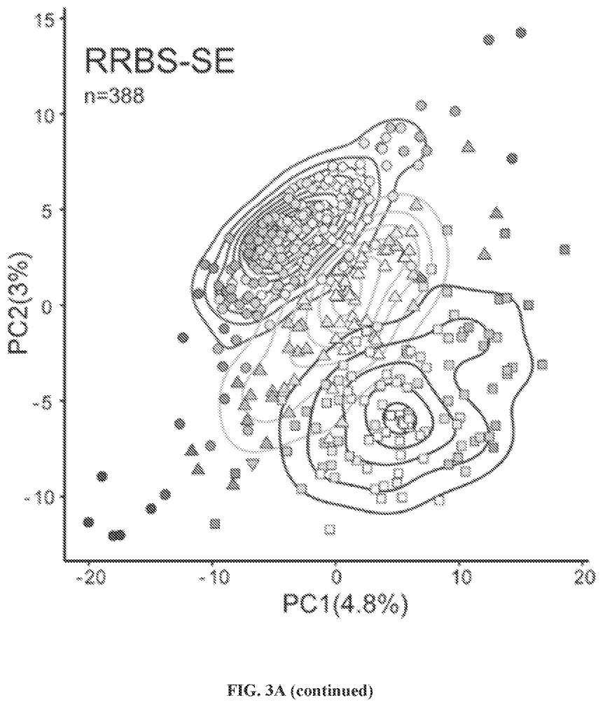

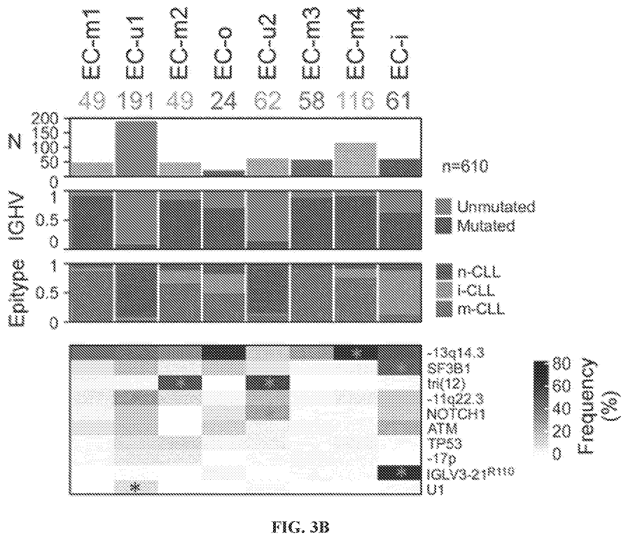

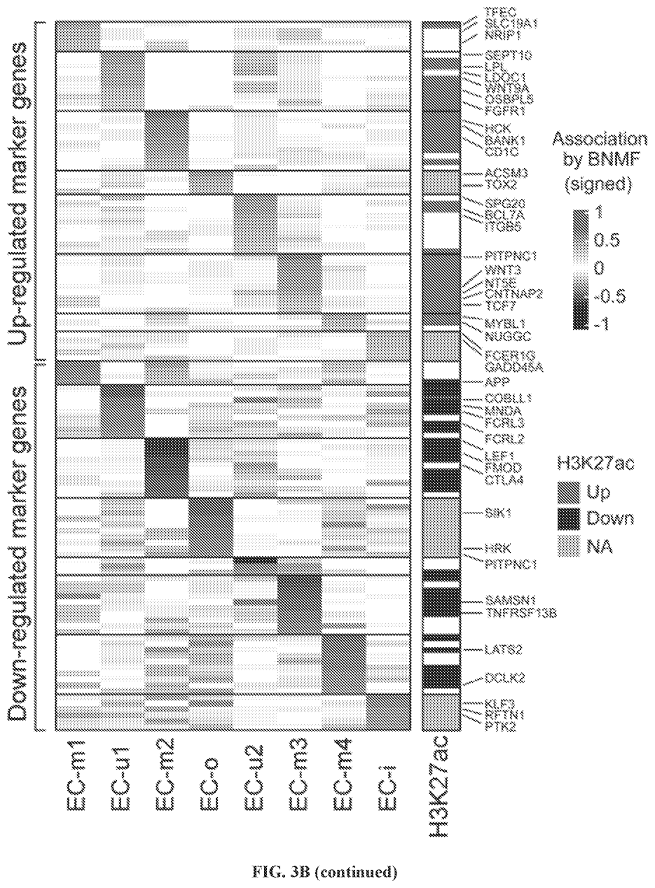

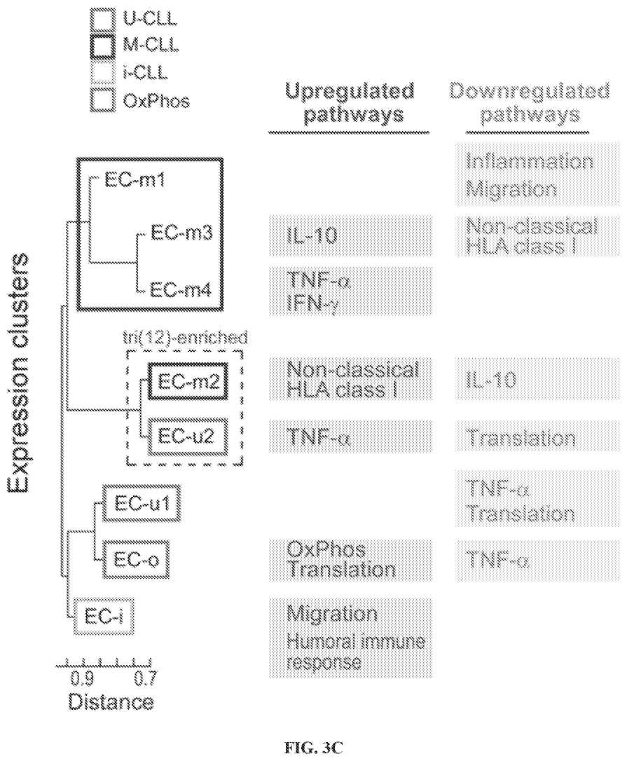

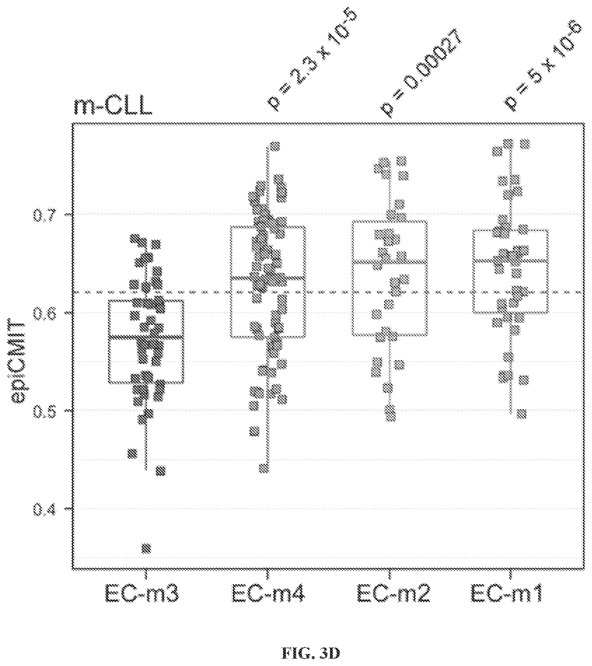

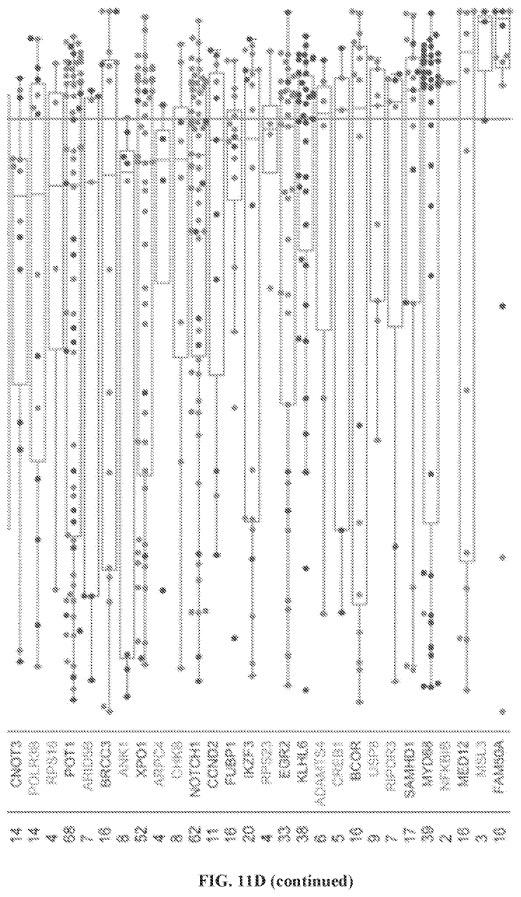

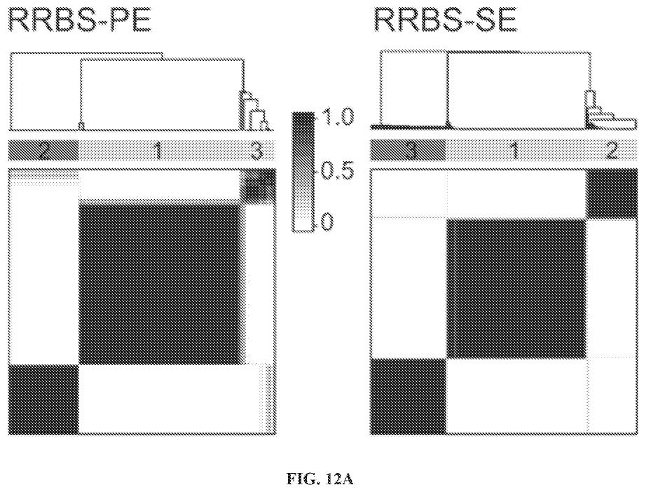

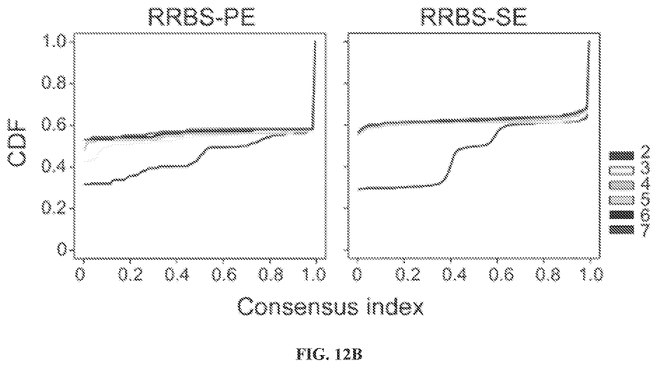

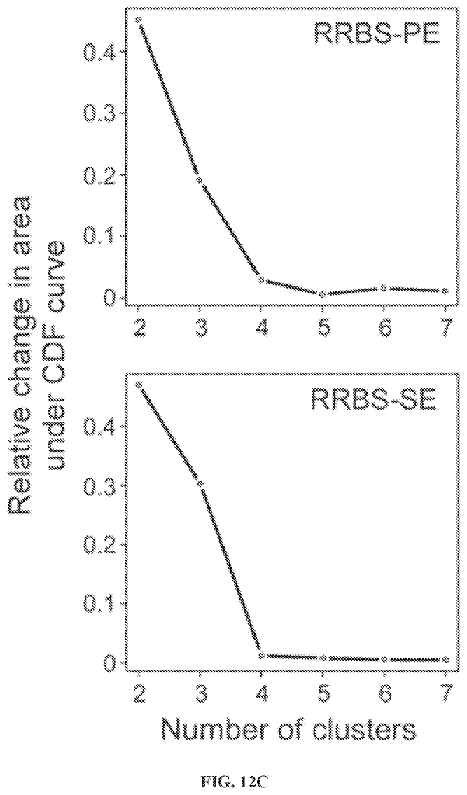

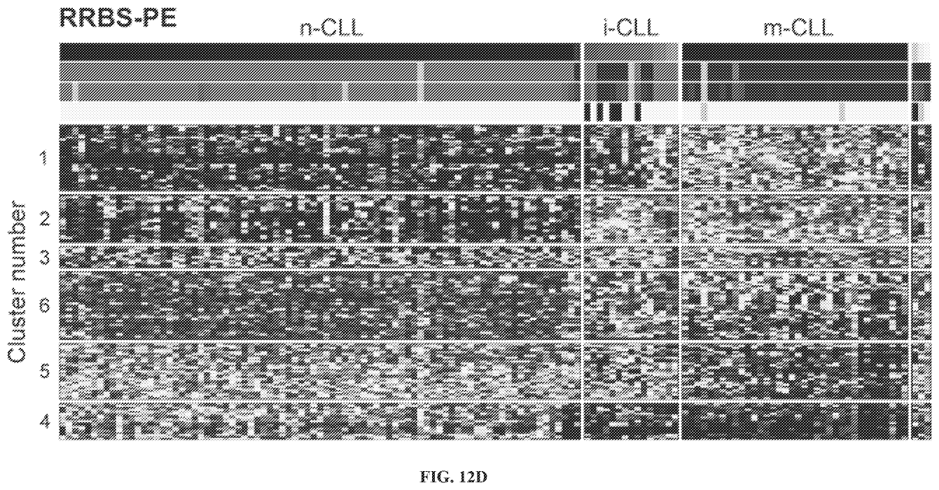

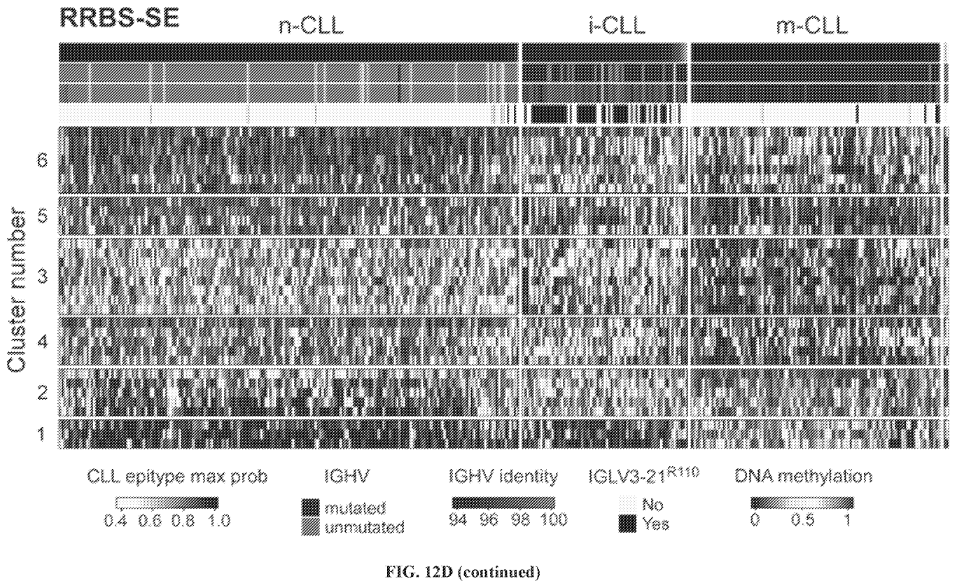

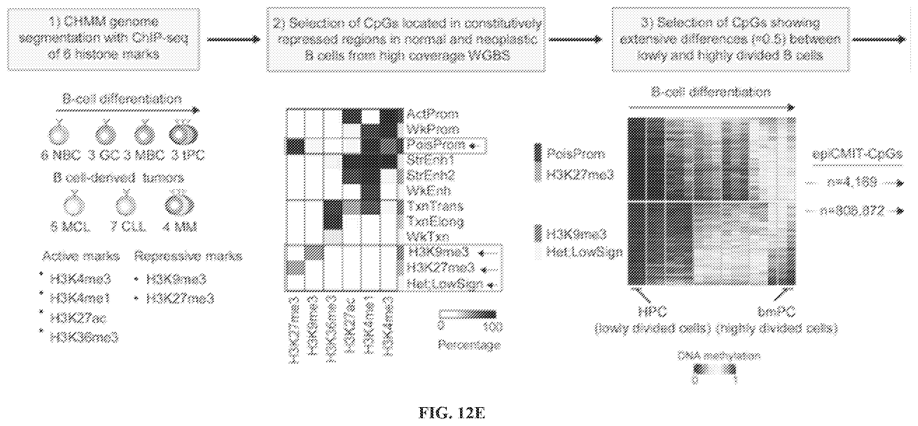

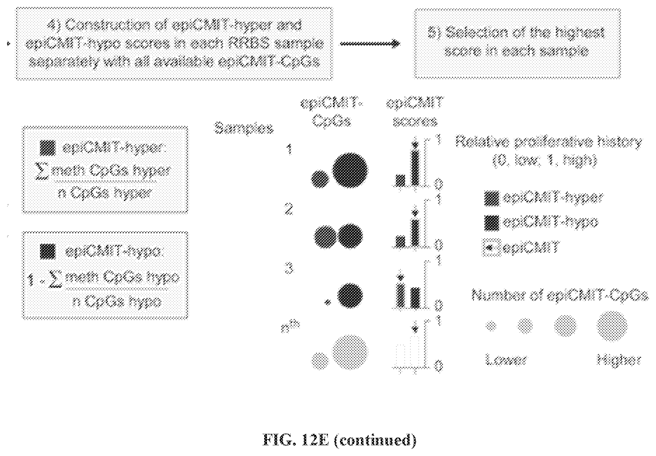

In addition to subtypes based on IGHV (heavy chain variable region of immunoglobulin genes) status, genome-wide DNA methylation studies previously identified three epigenetic groups (epitypes), defined based on distinct methylation profiles of pre- and post-germinal center experienced B cells: naive-like CLL (n-CLL, predominantly U-CLL), intermediate CLL (i-CLL, mix of M-CLL (CLL with mutated IGHV) and U-CLL), and memory-like CLL (m-CLL, predominantly M-CLL) (Oakes, C. C. et al. DNA methylation dynamics during B cell maturation underlie a continuum of disease phenotypes in chronic lymphocytic leukemia. Nat. Genet. 48, 253-264 (2016); Kulis, M. et al. Epigenomic analysis detects widespread gene-body DNA hypomethylation in chronic lymphocytic leukemia. Nat. Genet. 44, 1236-1242 (2012)). Furthermore, cell division likely results in epigenetic imprints that correlate with the proliferative history of the cell. A mitotic clock score called epigenetically-determined cumulative mitoses (epiCMIT) has further delineated prognosis within epitypes where higher epiCMIT scores corresponded with worse prognosis. Epitypes and epiCMIT were defined in previous studies using 450k DNA methylation arrays (n=490), but here new methodologies to incorporate reduced representation bisulfite sequencing data (RRBS) were developed and validated (n=509) (FIGS. 12A-12F). Evaluating the entire dataset (n=999), it was found that the two main sources of variation in the CLL DNA methylome were explained by components of cellular memory: the cell of origin (epitype) and the proliferative history of the cell (epiCMIT) (FIG. 3A).

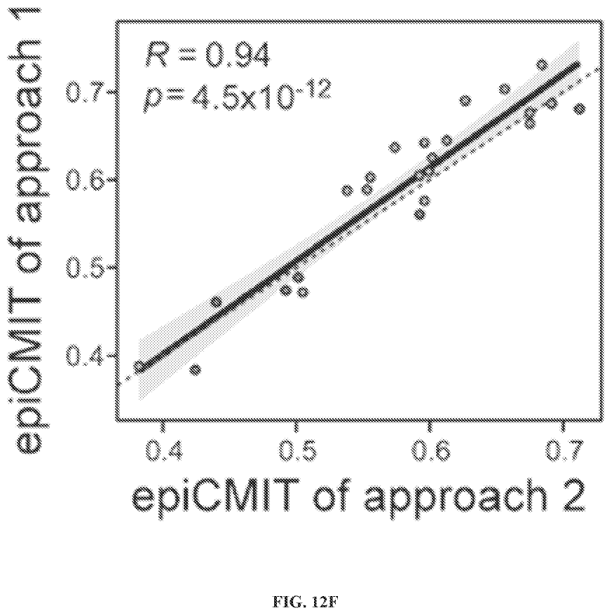

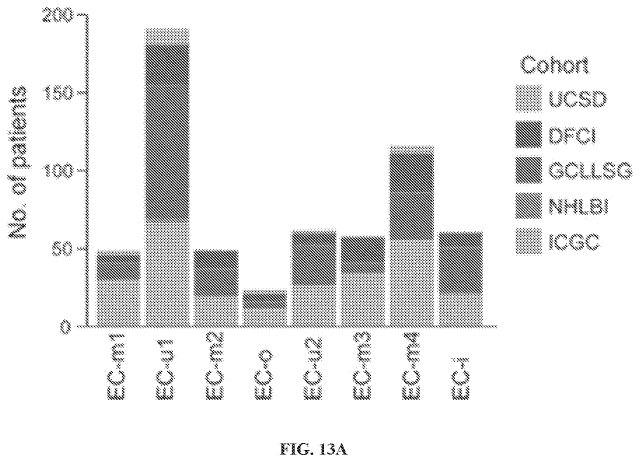

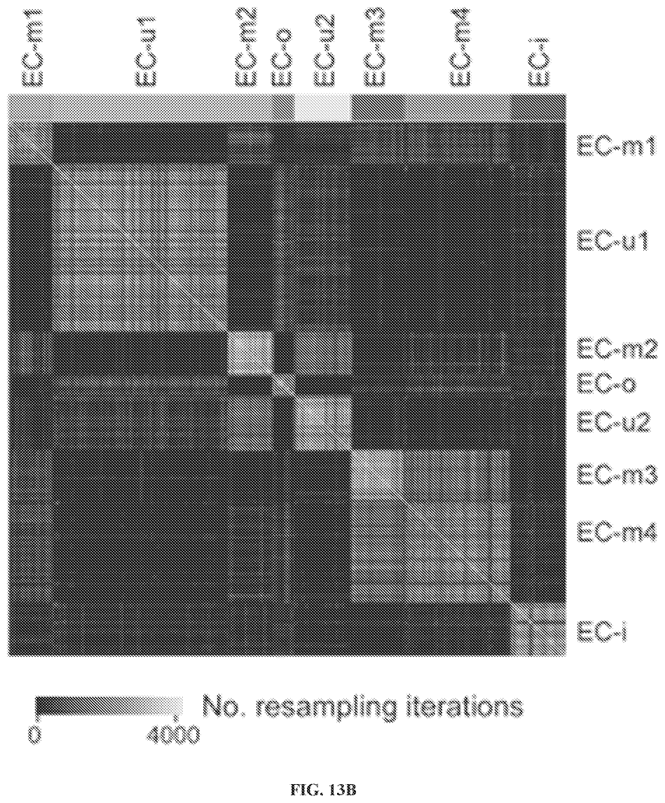

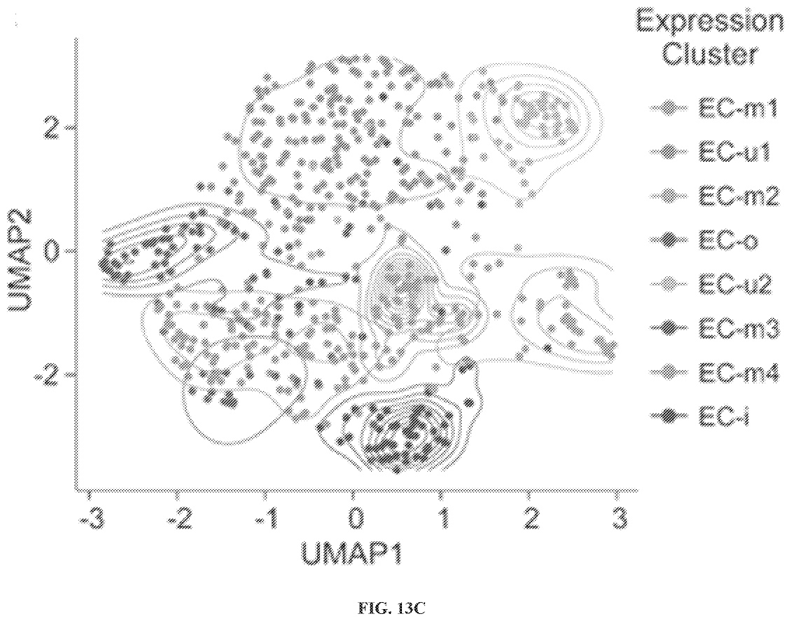

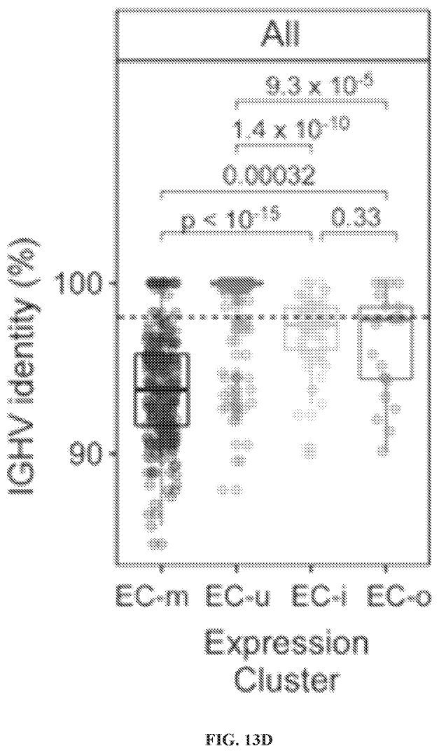

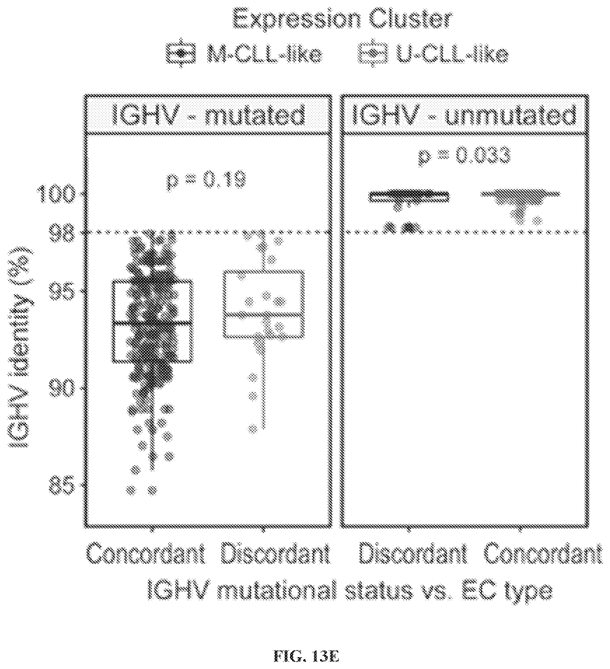

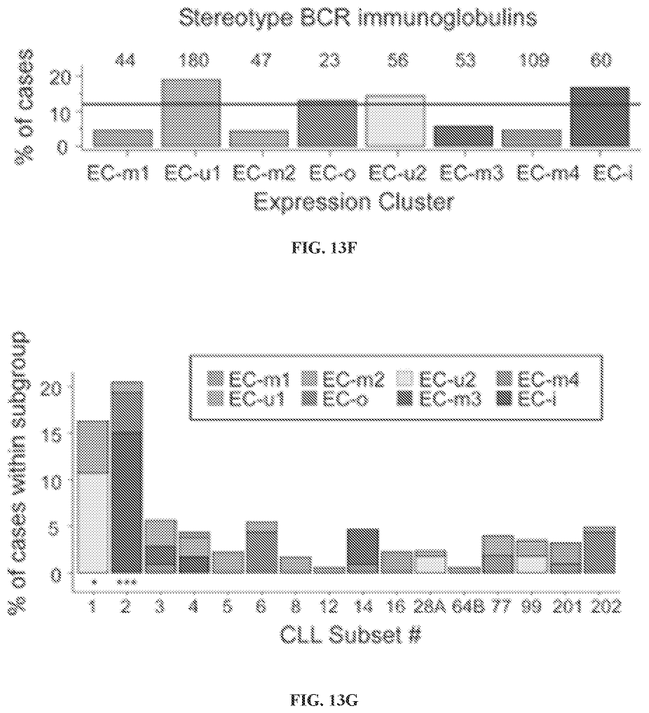

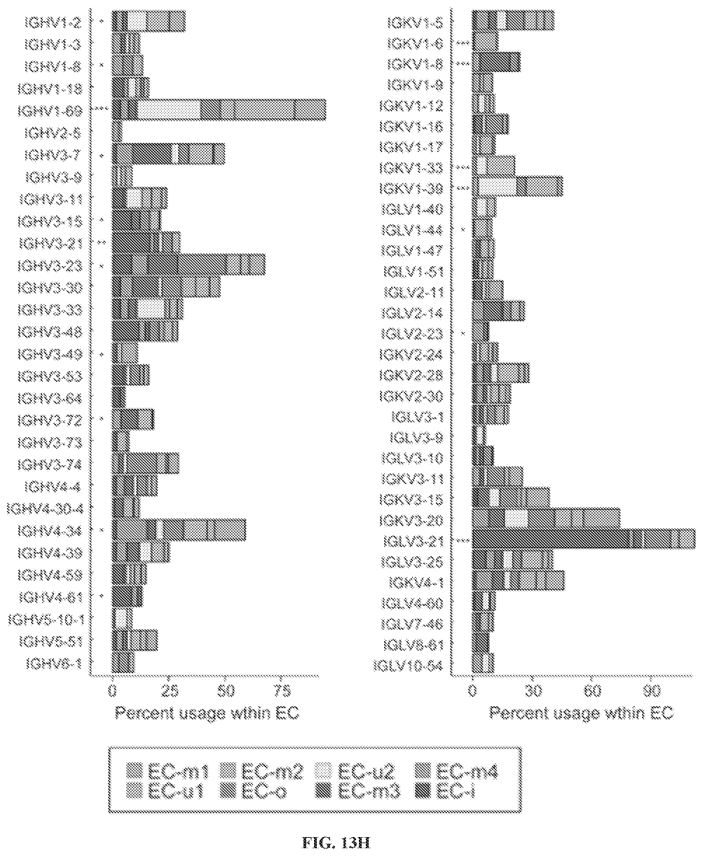

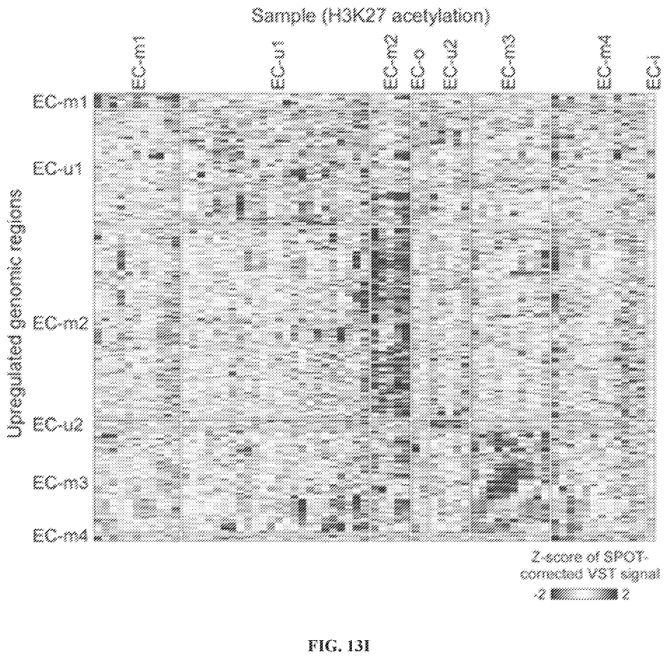

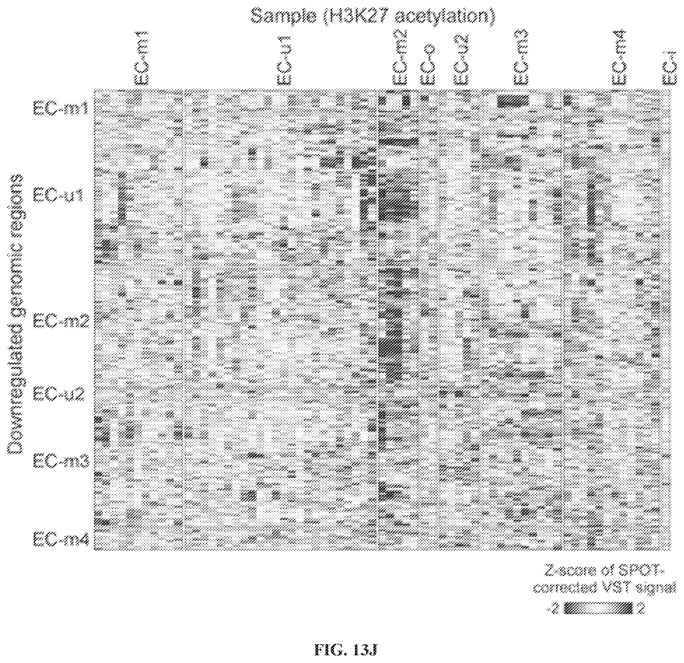

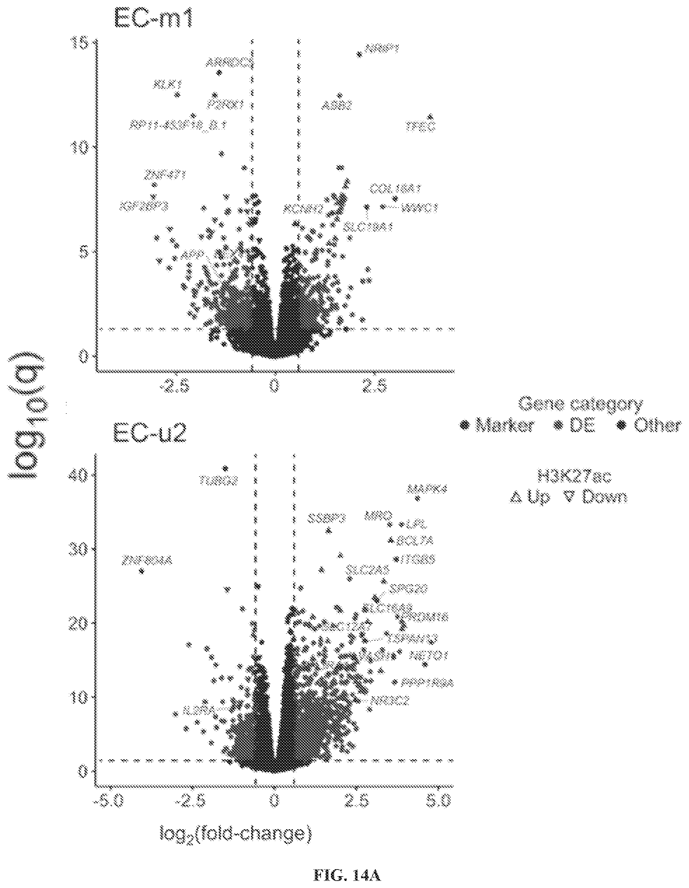

While the overall DNA methylome mainly reflects the cellular past of each CLL, the present phenotypic state can be determined by investigating transcriptomes. By applying Bayesian non-negative matrix factorization for unsupervised clustering of RNA-seq data from 610 treatment-naive CLL samples, 8 robust expression clusters (ECs) were identified (FIGS. 3B, and 13A-13C; Tables 3A, 3B, and 4). The expression clusters (ECs) strongly associated with IGHV (heavy chain variable region of immunoglobulin genes) mutational status and/or epitype, revealing two subtypes of U-CLL/n-CLL (EC-u1, EC-u2) and four subtypes of M-CLL/m-CLL (EC-m1, EC-m2, EC-m3, and EC-m4) (Tables 3A, 3B, and 4). EC-i was best defined by the i-CLL epitype whereas EC-o, the smallest cluster (n=24; 3.9%), was not significantly associated with any previously defined CLL group. Both EC-i and EC-o displayed borderline identity of somatic hypermutations in IGHV (heavy chain variable region of immunoglobulin genes) with germline, close to the 98% threshold distinguishing M-CLL (CLL with mutated IGHV) from U-CLL (CLL with unmutated IGHV) (FIG. 13D).

Tables 3, 3B, and 4 relate to the Expression cluster (EC) analysis.

TABLE 3A
Expression cluster (EC) marker genes determined
by non-negative matrix factorization

| | | | | GENE | UNIPROT | ENTREZ |
|---|---|---|---|---|---|---|
| GENE | EC | DIRECTION | RANK | SYNONYM | ID | GENE ID |
| TFEC | EC-m1 | UP | 1 | | O14948 | 22797 |
| COL18A1 | EC-m1 | UP | 2 | | P39060 | 80781 |
| SLC19A1 | EC-m1 | UP | 3 | | P41440 | 6573 |
| NRIP1 | EC-m1 | UP | 4 | | P48552 | 8204 |
| KCNH2 | EC-m1 | UP | 5 | | Q12809 | 3757 |
| SEPT10 | EC-u1 | UP | 1 | | | |
| LDOC1 | EC-u1 | UP | 2 | | O95751 | 23641 |
| LPL | EC-u1 | UP | 3 | | P06858 | 4023 |
| KANK2 | EC-u1 | UP | 4 | | Q63ZY3 | 25959 |
| SOWAHC | EC-u1 | UP | 5 | | Q53LP3 | 65124 |
| DUSP26 | EC-u1 | UP | 6 | | Q9BV47 | 78986 |
| OSBPL5 | EC-u1 | UP | 7 | | Q9H0X9 | 114879 |
| WNT9A | EC-u1 | UP | 8 | | O14904 | 7483 |
| FGFR1 | EC-u1 | UP | 9 | | P11362 | 2260 |
| GTSF1L | EC-u1 | UP | 10 | | Q9H1H1 | 149699 |
| EML6 | EC-m2 | UP | 1 | | Q6ZMW3 | 400954 |
| HCK | EC-m2 | UP | 2 | | P08631 | 3055 |
| CD1C | EC-m2 | UP | 3 | | P29017 | 911 |
| VPS37B | EC-m2 | UP | 4 | | Q9H9H4 | 79720 |
| CYBB | EC-m2 | UP | 5 | | P04839 | 1536 |
| NXPH4 | EC-m2 | UP | 6 | | O95158 | 11247 |
| BTNL9 | EC-m2 | UP | 7 | | Q6UXG8 | 153579 |
| KLRK1 | EC-m2 | UP | 8 | | P26718 | 100528032 |
| IQSEC1 | EC-m2 | UP | 9 | | Q6DN90 | 9922 |
| BANK1 | EC-m2 | UP | 10 | | Q8NDB2 | 55024 |
| ACSM3 | EC-o | UP | 1 | | Q53FZ2 | 6296 |
| TOX2 | EC-o | UP | 2 | | Q96NM4 | 84969 |
| PHF16 | EC-o | UP | 3 | JADE3 | | |
| SESN3 | EC-o | UP | 4 | | P58005 | 143686 |
| ITGB5 | EC-u2 | UP | 1 | | P18084 | 3693 |
| BCL7A | EC-u2 | UP | 2 | | Q4VC05 | 605 |
| PPP1R9A | EC-u2 | UP | 3 | | Q9ULJ8 | 55607 |
| TSPAN13 | EC-u2 | UP | 4 | | O95857 | 27075 |
| SLC12A7 | EC-u2 | UP | 5 | | Q9Y666 | 10723 |
| SSBP3 | EC-u2 | UP | 6 | | Q9BWW4 | 23648 |
| VASH1 | EC-u2 | UP | 7 | | Q7L8A9 | 22846 |
| SPG20 | EC-u2 | UP | 8 | SPART | | |
| IL13RA1 | EC-u2 | UP | 9 | | P78552 | 3597 |
| NR3C2 | EC-u2 | UP | 10 | | P08235 | 4306 |
| MS4A4E | EC-m3 | UP | 1 | | Q96PG1 | 643680 |
| MYL9 | EC-m3 | UP | 2 | | P24844 | 10398 |
| NT5E | EC-m3 | UP | 3 | | P21589 | 4907 |
| MS4A6A | EC-m3 | UP | 4 | | Q9H2W1 | 64231 |
| PITPNC1 | EC-m3 | UP | 5 | | Q9UKF7 | 26207 |
| CNTNAP2 | EC-m3 | UP | 6 | | Q9UHC6 | 26047 |
| IGF2BP3 | EC-m3 | UP | 7 | | O00425 | 10643 |
| WNT3 | EC-m3 | UP | 8 | | P56703 | 101929777 |
| CLDN7 | EC-m3 | UP | 9 | | O95471 | 1366 |
| TCF7 | EC-m3 | UP | 10 | | P36402 | 6932 |
| MYBL1 | EC-m4 | UP | 1 | | P10243 | 4603 |
| NUGGC | EC-m4 | UP | 2 | | Q68CJ6 | 389643 |
| GNG8 | EC-m4 | UP | 3 | | Q9UK08 | 94235 |
| GRIK3 | EC-i | UP | 1 | | Q13003 | 2899 |
| IQGAP2 | EC-i | UP | 2 | | Q13576 | 10788 |
| FCER1G | EC-i | UP | 3 | | P30273 | 2207 |
| STK32B | EC-i | UP | 4 | | Q9NY57 | 55351 |
| GADD45A | EC-i | UP | 5 | | P24522 | 1647 |
| P2RX1 | EC-m1 | DOWN | 1 | | P51575 | 5023 |
| ARRDC5 | EC-m1 | DOWN | 2 | | A6NEK1 | 645432 |
| BEX4 | EC-m1 | DOWN | 3 | | Q9NWD9 | 56271 |
| APP | EC-m1 | DOWN | 4 | | P05067 | 351 |
| ADD3 | EC-u1 | DOWN | 1 | | Q9UEY8 | 120 |
| AKT3 | EC-u1 | DOWN | 2 | | Q9Y243 | 10000 |
| COBLL1 | EC-u1 | DOWN | 3 | | Q53SF7 | 22837 |
| MNDA | EC-u1 | DOWN | 4 | | P41218 | 4332 |
| FCRL3 | EC-u1 | DOWN | 5 | | Q96P31 | 115352 |
| FAM49A | EC-u1 | DOWN | 6 | CYRIA | | |
| FCRL2 | EC-u1 | DOWN | 7 | | Q96LA5 | 79368 |
| SLC2A3 | EC-u1 | DOWN | 8 | | P11169 | 6515 |
| MARCKS | EC-u1 | DOWN | 9 | | P29966 | 4082 |
| LEF1 | EC-m2 | DOWN | 1 | | Q9UJU2 | 51176 |
| SH3D21 | EC-m2 | DOWN | 2 | | A4FU49 | 79729 |
| FMOD | EC-m2 | DOWN | 3 | | Q06828 | 2331 |
| SEMA4A | EC-m2 | DOWN | 4 | | Q9H3S1 | 64218 |
| CTLA4 | EC-m2 | DOWN | 5 | | P16410 | 1493 |
| ADTRP | EC-m2 | DOWN | 6 | | Q96IZ2 | 84830 |
| IGSF3 | EC-m2 | DOWN | 7 | | O75054 | 3321 |
| IGFBP4 | EC-m2 | DOWN | 8 | | P22692 | 3487 |
| PDGFD | EC-m2 | DOWN | 9 | | Q9GZP0 | 80310 |
| APOD | EC-m2 | DOWN | 10 | | P05090 | 347 |
| TBC1D9 | EC-o | DOWN | 1 | | Q6ZT07 | 23158 |
| PIP5K1B | EC-o | DOWN | 2 | | O14986 | 8395 |
| SIK1 | EC-o | DOWN | 3 | | P57059 | 150094 |
| DUSP5 | EC-o | DOWN | 4 | | Q16690 | 1847 |
| GNG7 | EC-o | DOWN | 5 | | O60262 | 2788 |
| HIVEP3 | EC-o | DOWN | 6 | | Q5T1R4 | 59269 |
| MARCKSL1 | EC-o | DOWN | 7 | | P49006 | 65108 |
| GPR183 | EC-o | DOWN | 8 | | P32249 | 1880 |
| HRK | EC-o | DOWN | 9 | | O00198 | 8739 |
| PITPNC1 | EC-o | DOWN | 10 | | Q9UKF7 | 26207 |
| TUBG2 | EC-u2 | DOWN | 1 | | Q9NRH3 | 27175 |
| ZNF804A | EC-u2 | DOWN | 2 | | Q7Z570 | 91752 |
| IL2RA | EC-u2 | DOWN | 3 | | P01589 | 3559 |
| BASP1 | EC-m3 | DOWN | 1 | | P80723 | 10409 |
| FLJ20373 | EC-m3 | DOWN | 2 | | | |
| MAP4K4 | EC-m3 | DOWN | 3 | | O95819 | 9448 |
| LRRK2 | EC-m3 | DOWN | 4 | | Q5S007 | 120892 |
| SAMSN1 | EC-m3 | DOWN | 5 | | Q9NSI8 | 388813 |
| CEACAM1 | EC-m3 | DOWN | 6 | | P13688 | 634 |
| TNFRSF13B | EC-m3 | DOWN | 7 | | O14836 | 23495 |
| PHF16 | EC-m3 | DOWN | 8 | JADE3 | | |
| MID1IP1 | EC-m3 | DOWN | 9 | | Q9NPA3 | 58526 |
| ABCA9 | EC-m3 | DOWN | 10 | | Q8IUA7 | 10350 |
| AEBP1 | EC-m4 | DOWN | 1 | | Q8IUX7 | 165 |
| HIP1R | EC-m4 | DOWN | 2 | | O75146 | 9026 |
| LATS2 | EC-m4 | DOWN | 3 | | Q9NRM7 | 26524 |
| RIMKLB | EC-m4 | DOWN | 4 | | Q9ULI2 | 57494 |
| EML6 | EC-m4 | DOWN | 5 | | Q6ZMW3 | 400954 |
| FADS3 | EC-m4 | DOWN | 6 | | Q9Y5Q0 | 3995 |
| MBOAT1 | EC-m4 | DOWN | 7 | | Q6ZNC8 | 154141 |
| LCN10 | EC-m4 | DOWN | 8 | | Q6JVE6 | 414332 |
| DCLK2 | EC-m4 | DOWN | 9 | | Q8N568 | 166614 |
| GLUL | EC-m4 | DOWN | 10 | | P15104 | 2752 |
| ITGAX | EC-i | DOWN | 1 | | P20702 | 3687 |
| KLF3 | EC-i | DOWN | 2 | | P57682 | 51274 |
| RFTN1 | EC-i | DOWN | 3 | | Q14699 | 23180 |
| PTK2 | EC-i | DOWN | 4 | | Q05397 | 5747 |
| DFNB31 | EC-i | DOWN | 5 | | | |
| ZMAT1 | EC-i | DOWN | 6 | | Q5H9K5 | 84460 |

TABLE 3B
Legend for Table 3A

| Column | Description |
|---|---|
| GENE | Marker gene |
| EC | Expression cluster |
| RANK | Rank of marker gene per this EC |
| GENE | Gene symbol synonym (more |
| SYNONYM | updated) |
| UNIPROT ID | Uniprot protein accession ID |
| ENTREZ | Entrez database gene accession |
| GENE ID | ID |

TABLE 4
Biomarkers used in expression cluster (EC) classifier

| | BATCH |
|---|---|
| | CORRECTED |
| TRANSCRIPTS | TRANSCRIPTS |
| PER MILLION | PER MILLION |
| (TPM) | (TPM) |
| CLASSIFIER | CLASSIFIER |
| BIOMARKERS | BIOMARKERS |
| ABCA9 | ACAP3 |
| ACAP3 | ACSM3 |
| ACSM3 | AEBP1 |
| ADAP2 | AKT3 |
| AF127936.7 | ARHGAP33 |
| ARHGAP33 | ARHGAP42 |
| ARMC7 | ARMC7 |
| ARRDC5 | ARRDC5 |
| ARSD | ATPIF1 |
| ARSI | BACH2 |
| ASB2 | BASP1 |
| ATP1A3 | BCL7A |
| ATP2B1 | C17orf100 |
| ATPIF1 | CBLB |
| BASP1 | CD72 |
| BCL2A1 | CD86 |
| BCL7A | CEACAM1 |
| BCS1L | CHPT1 |
| CAMK2A | CLDN7 |
| CLDN23 | CMTM7 |
| CMTM7 | CNTNAP1 |
| COBLL1 | COBLL1 |
| CRELD2 | COL18A1 |
| CRY1 | CRY1 |
| CTAGE9 | CTLA4 |
| CTLA4 | EGR3 |
| DDR1 | EML6 |
| DKFZP761J1410 | EZH2 |
| DPF3 | FADS3 |
| EML6 | FCER1G |
| ERRFI1 | FCRL2 |
| ESPNL | FGL2 |
| EZH2 | FLJ20373 |
| FAHD2B | FMOD |
| FAM109A | GADD45A |
| FBXO27 | GLIPR1 |
| FGL2 | GNB4 |
| FLJ20373 | GPR160 |
| FMOD | GPR34 |
| GADD45A | GRIK3 |
| GNAO1 | GUCD1 |
| GPR160 | HCK |
| GPR34 | HIP1R |
| GUCD1 | HIVEP3 |
| HCK | HMCES |
| HDAC4 | IGF2BP3 |
| HIP1R | IGSF3 |
| HMCES | IL21R |
| IGSF3 | INPP5F |
| IQSEC1 | IQGAP2 |
| ITGAX | IQSEC1 |
| KCNH3 | ITGAX |
| KCNN3 | ITGB5 |
| KCTD3 | JDP2 |
| KDM1B | KANK2 |
| KLK1 | KCNH2 |
| KSR1 | KDM1B |
| LCN10 | KLF3 |
| LINC00865 | LATS2 |
| LPL | LCN10 |
| LRRK2 | LEF1 |
| LUZP1 | LPL |
| MAP4K4 | LRRK2 |
| MAPK4 | LUZP1 |
| MAST4 | MAP4K4 |
| MPRIP | MID1IP1 |
| MRO | MMP14 |
| MSI2 | MPRIP |
| MVB12B | MSI2 |
| MYBL1 | MYBL1 |
| MYC | MYL9 |
| MYL5 | MYLIP |
| MYL9 | MZB1 |
| MYO3A | NBPF3 |
| NEDD9 | NRIP1 |
| NFKBIZ | NRSN2 |
| NR2F6 | NUGGC |
| NRIP1 | NXPH4 |
| NRSN2 | P2RX1 |
| NUGGC | P2RX5 |
| P2RX1 | P2RY14 |
| PELI3 | PDGFD |
| PIGB | PIP5K1B |
| PIP5K1B | PITPNC1 |
| PITPNC1 | PON2 |
| PLD1 | PRICKLE1 |
| PTPN7 | PTPN7 |
| QDPR | RCN3 |
| REPS2 | RDX |
| RHBDF2 | RHBDF2 |
| RIMKLB | RIMKLB |
| RP11-134N1.2 | RNF135 |
| RP11-265P11.1 | RP11-145M9.4 |
| RP11-453F18_B.1 | RP11-268J15.5 |
| RP11-456H18.2 | RP11-463O12.3 |
| RP1-90J20.12 | RP5-1028K7.2 |
| SAMSN1 | SAMSN1 |
| SCPEP1 | SCCPDH |
| SH3D21 | SCD |
| SLC44A1 | SCPEP1 |
| SLC4A7 | SDC3 |
| SLC4A8 | SECTM1 |
| SMIM10 | SESN3 |
| SPN | SH3BP2 |
| SSBP3 | SH3D21 |
| STAM | SLC16A5 |
| STX5 | SLC19A1 |
| SYNGR3 | SLC4A7 |
| TAS1R3 | SPN |
| TBC1D2B | SSBP3 |
| TBC1D9 | STX5 |
| TFEC | SUSD1 |
| TIMELESS | TBC1D2B |
| TNFRSF13B | TBC1D9 |
| TNR | TBKBP1 |
| TOX2 | TCF7 |
| TRIM7 | TFEC |
| TUBG2 | TGFBR3 |
| VSIG10 | TIGIT |
| WNT5A | TIMELESS |
| ZMYND8 | TMEM133 |
| ZNF804A | TNFRSF13B |
| | TOX2 |
| | TRAK2 |
| | TTC39C |
| | TUBG2 |
| | VPS37B |
| | VSIG10 |
| | WNT9A |
| | ZAP70 |
| | ZNF667-AS1 |
| | ZNF804A |
| | ZSWIM6 |

Of note, 8% of samples had discordant IGHV (heavy chain variable region of immunoglobulin genes) status and expression cluster (EC) assignment (i.e., M-CLLs included in EC-u clusters or vice versa). As an example of these discordant cases, it was observed that 11 M-CLLs clustered in EC-u2, comprising 17.7% of this EC-u cluster. IGHV (heavy chain variable region of immunoglobulin genes) mutation rate for discordant cases was compared to those with concordant expression profiles, and while a small difference in mean percent identity in U-CLL (CLL with unmutated IGHV) was detected (t-test p=0.033, 99.67% versus 99.96% means, respectively), no difference was found among M-CLL (CLL with mutated IGHV) cases (p=0.19, 93.98% versus 93.23%) (FIG. 13E). Although correctly classified, some discordant cases had borderline IGHV (heavy chain variable region of immunoglobulin genes) status (97.5-98.5% IGHV identity; n=8) consistent with enrichment of the i-CLL epitype (18.6% in discordant vs. 6.65% in concordant samples, Fisher's Exact test p=0.012). Interestingly, CHD2 alterations were overrepresented in discordant M-CLL (CLL with mutated IGHV) cases where 42.9% had either CHD2 mutation or loss of 15q26.1 encompassing CHD2 (Fisher's Exact test p=0.002).

It was further explored whether the expression clusters (ECs) were enriched with specific driver events. Indeed, EC-u1 was associated with loss of 11q22.3 and U1 mutations, whereas EC-u2 displayed enrichment of NOTCH1 mutations and tri(12) (q<0.1) (FIG. 3B). EC-m2 was also associated with tri(12), occurring in 54.3%. SF3B1 and IGLV3-21^{R110} mutations were both enriched in EC-i (52.5% and 78.7%, respectively), which is consistent with previous work demonstrating their association with the i-CLL epitype. Conversely, EC-m1 was enriched with driverless patients (25% of M-CLLs, Fisher's Exact test q=0.006, odds-ratio 4.86). In addition to assessing genetic alterations, analyses were done to determine which expression clusters (ECs) displayed major stereotyped immunoglobulin genes, which are found in 13.5% of CLL and are divided into subsets that are associated with clinical outcome. All EC-m clusters had a lower proportion of major stereotyped B cell receptors (BCRs, 4.3-5.6%), whereas there was a higher incidence in the other expression clusters (ECs) (13-18.9%) (FIG. 13F). EC-i was significantly associated with CLL stereotyped subset #2 and IGLV3-21 gene expression consistent with previous findings that this stereotyped subset contains IGLV3-21^{R110} mutations (FIGS. 13G and 13H).

To further explore the biological differences among the ECs, marker genes were identified that were significantly upregulated or downregulated and which were respectively supported by increased or decreased histone 3 lysine 27 acetylation levels (H3K27ac, a mark of active regulatory elements) (FIGS. 3B, 13I, and 13J). The top upregulated marker genes in EC-u1 included SEPT10 and LPL. Another upregulated EC-u1 gene, OSBPL5, is likely a top expression marker predicting shorter time to progression after treatment with fludarabine, cyclophosphamide, and rituximab.

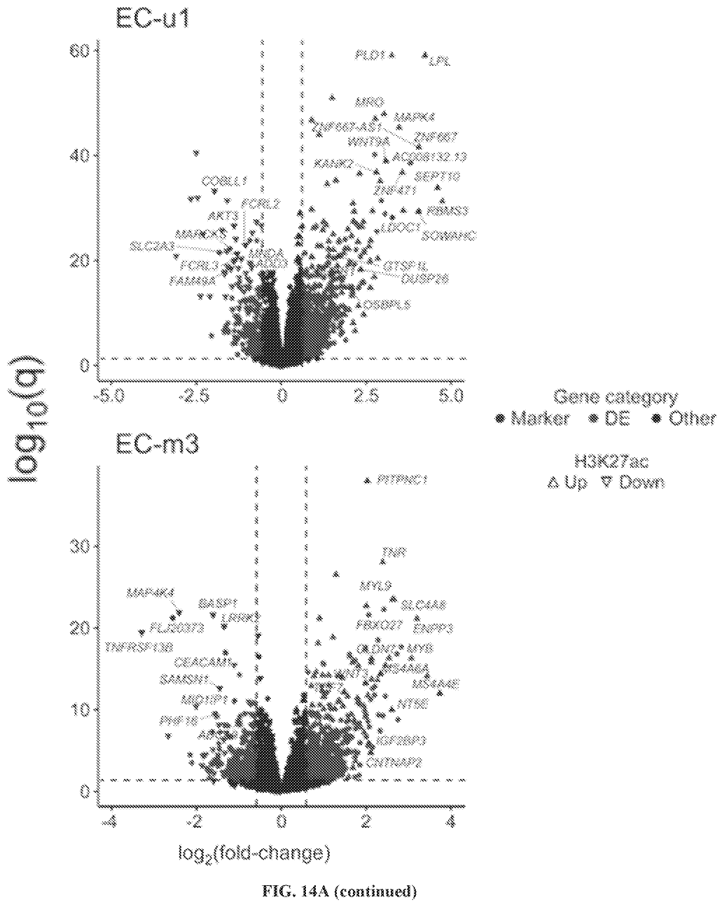

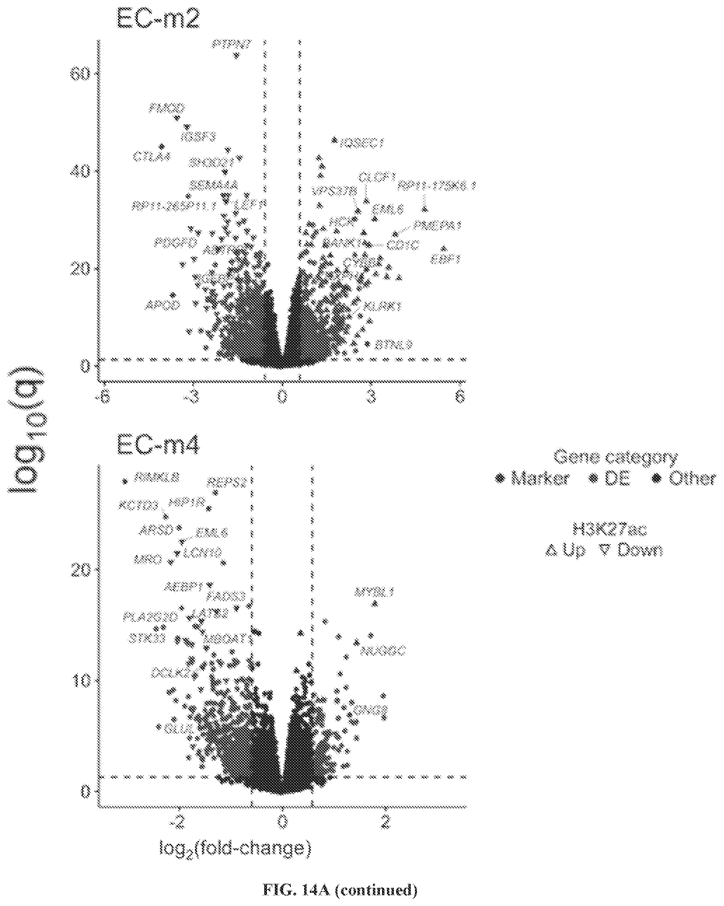

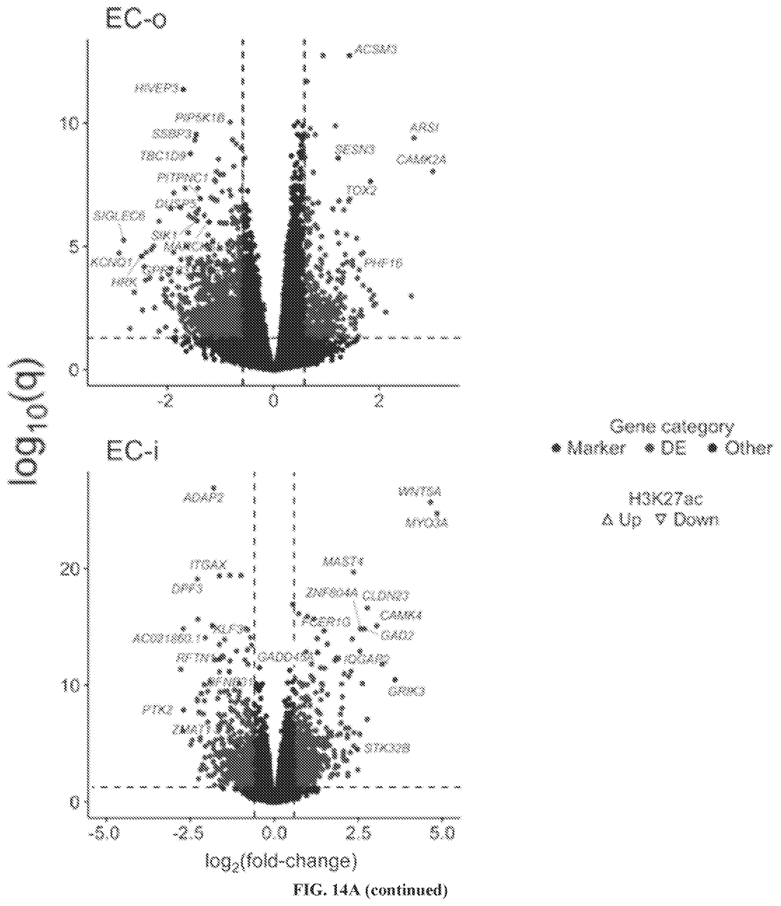

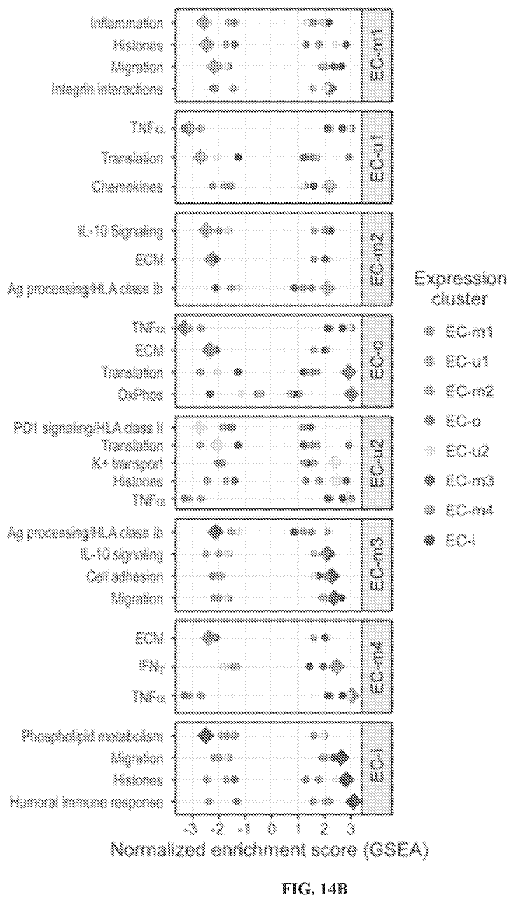

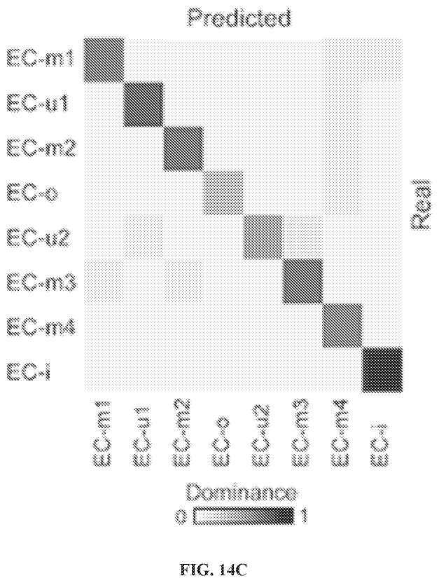

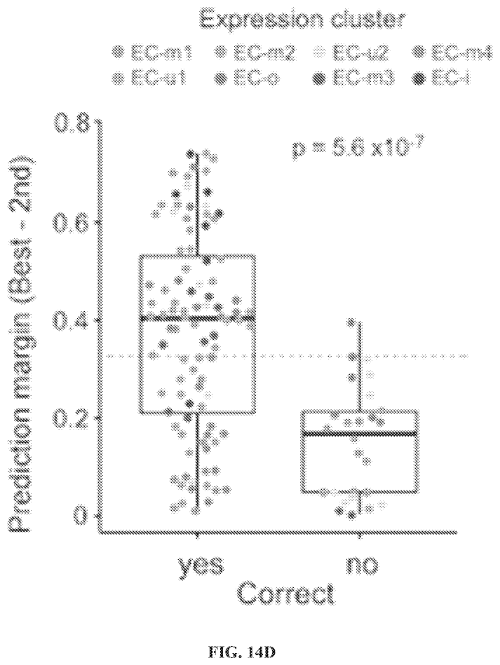

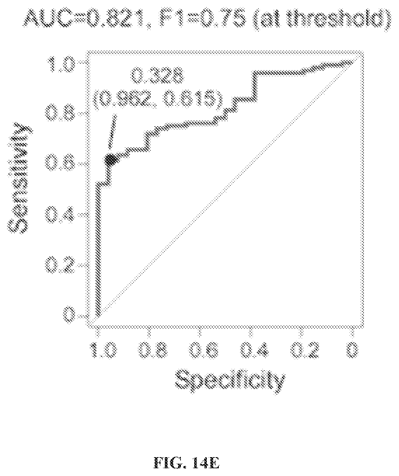

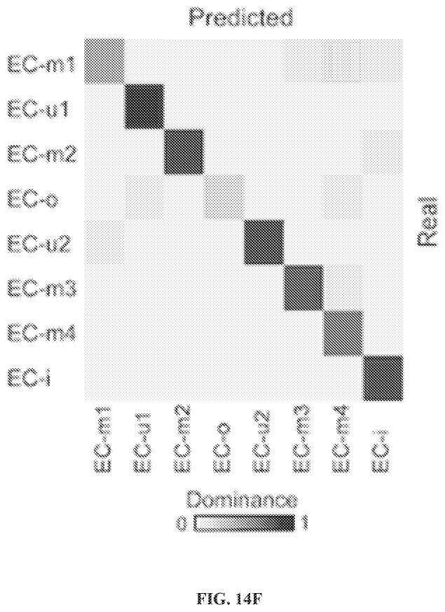

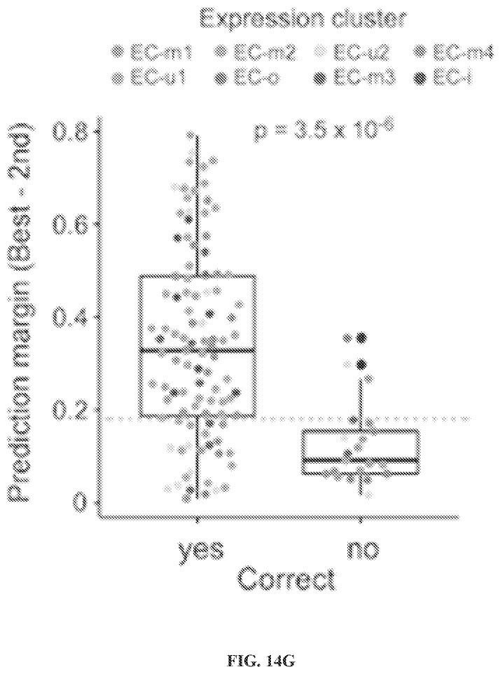

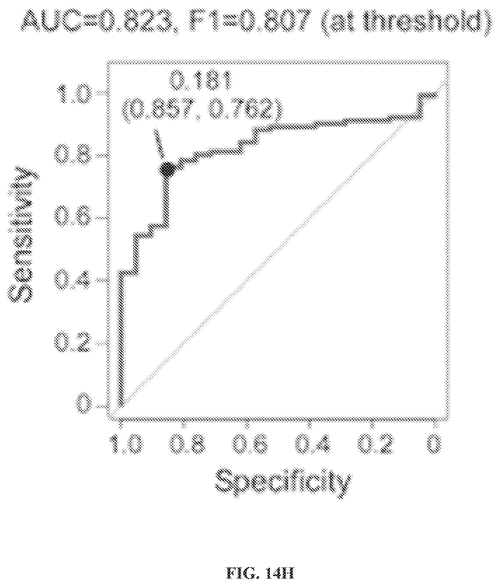

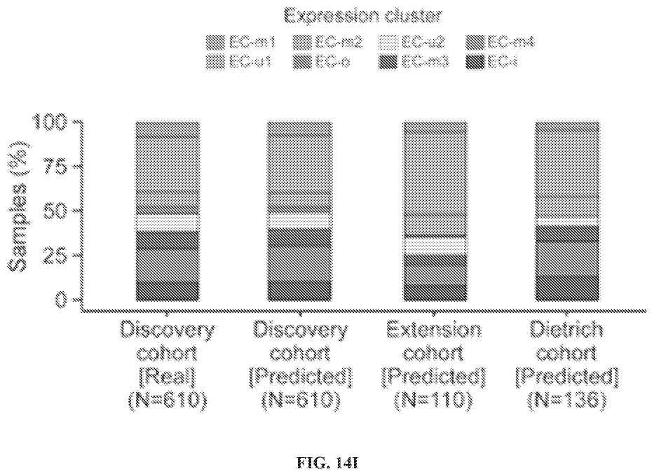

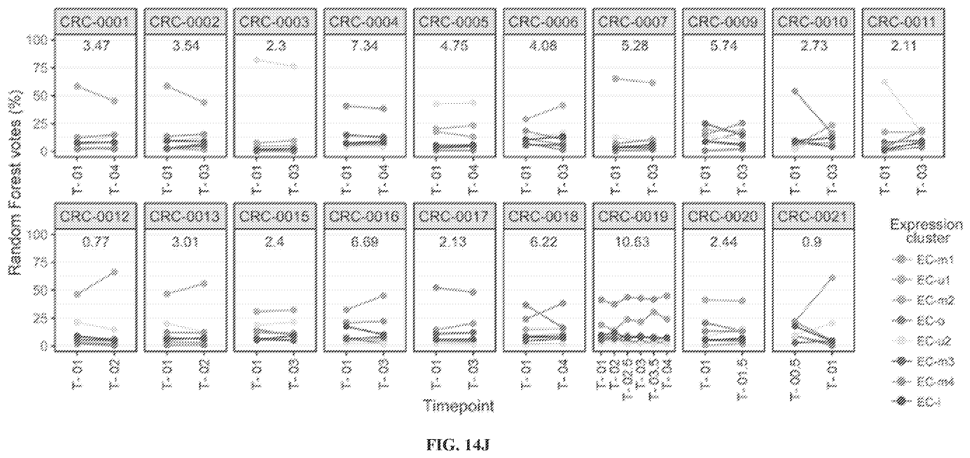

Differentially expressed genes in each expression cluster (EC) reflected heterogeneity in biological pathways that was captured by gene set enrichment analysis (FIGS. 3C, 14A, and 14B). Although EC-o was not associated with IGHV (heavy chain variable region of immunoglobulin genes) status or epitype, it was defined by enrichment in oxidative phosphorylation signaling relative to the other expression clusters (ECs) (q=4.24×10⁻¹³). The EC-m clusters were distinguished by either upregulated or downregulated inflammatory signaling or antigen expression via nonclassical HLA class I. The EC-u clusters shared gene expression changes reflecting impaired protein translation, but were differentiated by TNFα signaling, which was low in EC-u1 and high in EC-u2. EC-i was enriched for pathways regulating migration and the humoral immune response, possibly reflecting the autonomous BCR signaling of IGLV3-21^{R110}. Finally, the epiCMIT scores of the expression clusters (ECs) within each epitype were compared. In EC-m clusters, EC-m3 had a lower epiCMIT relative to the other ECs, consistent with a lower proliferative history and suggestive of better patient outcomes (FIG. 3D).

To evaluate the robustness of expression cluster (EC) classification and its potential application for prognostication in new samples, a classifier was built based on marker gene expression. It achieved 79% accuracy (83% after expression data batch correction), and when limiting predictions to high-confidence cases, attained 96% accuracy for 61.5% of patients (FIGS. 14C-14H). Applying the classifier to samples that were excluded from initial expression cluster (EC) discovery (n=110; 42.7% were post-treatment) and to an external CLL cohort (n=136) (Stilgenbauer, S. et al. Gene mutations and treatment outcome in chronic lymphocytic leukemia: results from the CLL8 trial. Blood 123, 3247-3254 (2014)) showed comparable expression cluster (EC) distributions (but a reduction in EC-o), supporting the relevance of these expression clusters (ECs) to new cohorts (FIG. 14I). Finally, by analyzing longitudinally sampled CLL specimens from 19 patients, expression cluster (EC) stability was confirmed over years of disease in most cases (p<1×10⁻⁶ by permutation, FIG. 14J). This provided further evidence that the expression clusters (ECs) are generally a stable readout and may also reflect clonal evolution, both of which are useful for prognostication.

### Example 5: Integrative Analysis Predicts Outcome

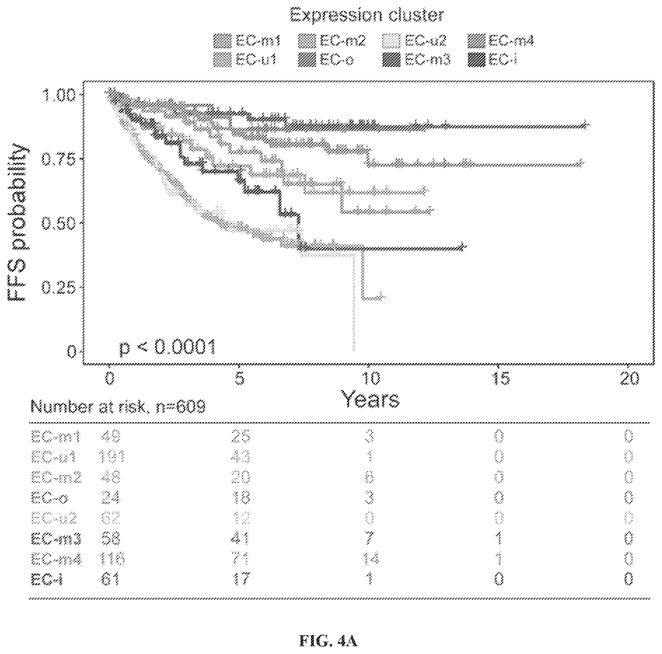

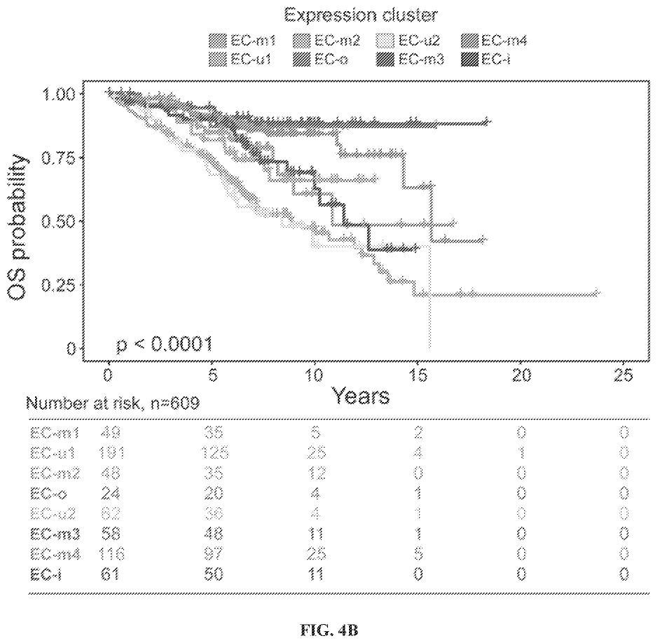

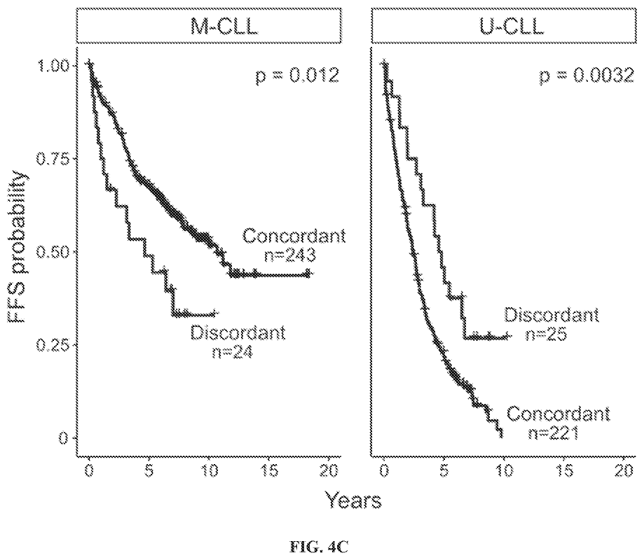

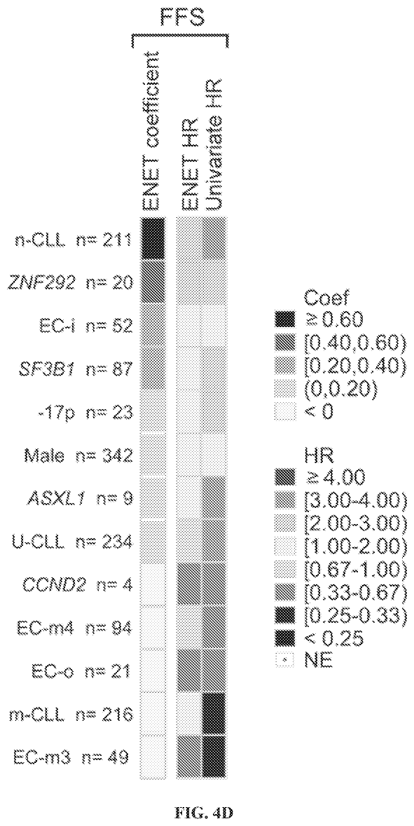

Multivariable analysis that included clinical features and IGHV (heavy chain variable region of immunoglobulin genes) status confirmed independent prognostic impact of the expression clusters (ECs) on failure free survival (FFS) (n=609, p<0.001) and overall survival (OS) (p=0.012) (Tables 5A-5D). The EC-u clusters had similarly short failure free survival (FFS) and EC-i displayed intermediate failure free survival (FFS) (FIG. 4A). However, outcomes in EC-m clusters were distinct where EC-m1, EC-m2, and EC-m4 demonstrated shorter failure free survival (FFS) relative to EC-m3, the cluster with best prognosis and lowest epiCMIT score. Differentiation of the EC-m clusters was also evident when evaluating overall survival (OS) (FIG. 4B). This confirmed expression clusters (ECs) as an independent prognostic factor in CLL, particularly in distinguishing prognosis in EC-m clusters.

TABLE 5A
Impact of expression clusters (ECs) on overall survival and
failure free survival

| | | 5 yr. overall | | 5 yr. failure | |
|---|---|---|---|---|---|
| | | survival | | free survival | |
| | | (OS) (%) | | (FFS) (%) | |
| | N (%) | [95% CI] | p-value | [95% CI] | p-value |
| N | 609 | 82 [79-85] | | 45 [41-49] | |
| Age | | | | | |
| <60 yrs. | 230 (38) | 88 [83-92] | 0.003 | 48 [42-55] | 0.51 |
| ≥60 yrs. | 379 (62) | 78 [74-82] | | 43 [38-48] | |
| Sex | | | | | |
| Male | 408 (67) | 80 [76-84] | 0.0001 | 40 [35-45] | <0.001 |
| Female | 201 (33) | 85 [79-90] | | 56 [48-62] | |
| IGHV status | | | | | |
| mutated | 322 (53) | 89 [85-92] | <0.001* | 63 [58-68] | <0.001* |
| unmutated | 275 (45) | 74 [68-79] | | 25 [20-30] | |
| unknown | 12 (2) | 82 [45-95] | | 31 [8-58] | |
| Expression | | | | | |
| Cluster | | | | | |
| EC-m1 | 49 (8) | 85 [70-92] | <0.001 | 61 [45-73] | <0.001 |
| EC-u1 | 191 (31) | 73 [66-79] | | 25 [19-32] | |
| EC-m2 | 48 (8) | 82 [67-91] | | 47 [32-61] | |
| EC-o | 24 (4) | 87 [65-96] | | 78 [56-90] | |
| EC-u2 | 62 (10) | 68 [54-79] | | 23 [13-35] | |
| EC-m3 | 58 (10) | 94 [84-98] | | 82 [68-90] | |
| EC-m4 | 116 (19) | 91 [84-95] | | 66 [56-74] | |
| EC-i | 61 (10) | 90 [79-95] | | 30 [19-42] | |

Median follow up time 7.5 years [95% CI, 7.3-7.8] for overall survival (OS) (#events 188/n = 609); failure free survival (FFS) (#events = 390/n = 609)
*Unknown IGHV (heavy chain variable region of immunoglobulin genes) status excluded

TABLE 5B
Cox regression modeling of overall survival

| | Univariate | | Multivariable | |
|---|---|---|---|---|
| | HR [95% CI] | p-value | HR [95% CI] | p-value |
| Age ≥60 yrs. | 1.59 [1.17- | 0.003 | 1.83 [1.34-2.51] | <0.001 |
| vs. <60 yrs. | 2.17] | | | |
| Sex, Male vs. | 1.96 [1.38- | <0.001 | 1.68 [1.18-2.40] | 0.004 |
| Female | 2.77] | | | |
| IGHV Mutation | | | | |
| Status | | | | |
| Unmutated vs | 3.00 [2.21- | <0.001 | 1.73 [1.07-2.81] | 0.027 |
| mutated | 4.08] | | | |
| Expression Cluster | | | | |
| EC-m1 vs. EC-m3 | 3.02 [1.15- | 0.025 | 2.93 [1.11-7.72] | 0.03 |
| | 7.95] | | | |
| EC-u1 vs. EC-m3 | 5.93 [2.59- | <0.001 | 4.16 [1.67-10.37] | 0.002 |
| | 13.57] | | | |
| EC-m2 vs. EC-m3 | 3.12 [1.18- | 0.021 | 3.03 [1.15-7.98] | 0.025 |
| | 8.20] | | | |
| EC-o vs. EC-m3 | 1.32 [0.33- | 0.69 | 1.41 [0.35-5.68 | 0.63 |
| | 5.28] | | | |
| EC-u2 vs. EC-m3 | 6.40 [2.65- | <0.001 | 4.38 [1.68-11.41] | 0.003 |
| | 15.47] | | | |
| EC-m4 vs. EC-m3 | 1.63 [0.65- | 0.3 | 1.86 [0.75-4.64] | 0.18 |
| | 4.06] | | | |
| EC-i vs. EC-m3 | 3.20 [1.27- | 0.014 | 2.62 [1.02-6.70] | 0.045 |
| | 8.08] | | | |

TABLE 5C
Cox regression modeling of failure free survival

| | Univariate | | Multivariable | |
|---|---|---|---|---|
| | HR [95% CI] | p-value | HR [95% CI] | p-value |
| Age ≥60 yrs. | 1.07 [0.87- | 0.52 | 1.22 [0.99-1.50] | 0.063 |
| vs. <60 yrs. | 1.32] | | | |
| Sex, Male vs. | 1.59 [1.27- | <0.001 | 1.44 [1.14-1.81] | 0.002 |
| Female | 1.99] | | | |
| IGHV Mutation | | | | |
| Status | | | | |
| Unmutated vs | 3.09 [2.50- | <0.001 | 1.66 [1.22-2.27] | 0.001 |
| mutated | 3.82] | | | |
| Unknown vs. | 2.31 [1.21- | 0.011 | 1.25 [0.63-2.47] | 0.52 |
| mutated | 4.39] | | | |
| Expression Cluster | | | | |
| EC-m1 vs. EC-m3 | 2.96 [1.55- | <0.001 | 2.86 [1.50-5.43] | 0.001 |
| | 5.62] | | | |
| EC-u1 vs. EC-m3 | 7.43 [4.28- | <0.001 | 5.17 [2.80-9.54] | <0.001 |
| | 12.91 | | | |
| EC-m2 vs. EC-m3 | 3.43 [1.80- | <0.001 | 3.24 [1.70-6.18] | <0.001 |
| | 6.52] | | | |
| EC-o vs. EC-m3 | 1.44 [0.60- | 0.42 | 1.39 [0.58-3.34] | 0.46 |
| | 3.42] | | | |
| EC-u2 vs. EC-m3 | 7.69 [4.25- | <0.001 | 5.50 [2.89-10.48] | <0.001 |
| | 13.91] | | | |
| EC-m4 vs. EC-m3 | 2.05 [1.14- | 0.017 | 2.14 [1.18-3.86] | 0.012 |
| | 3.70] | | | |
| EC-i vs. EC-m3 | 6.46 [3.55- | <0.001 | 5.57 [3.03-10.25] | <0.001 |
| | 11.75] | | | |

TABLE 5D
Model Comparison for ECs

| | −2 log Likelihood* | | |
|---|---|---|---|
| | | age, sex, | Chi squared test |
| Outcome | age, sex, IGHV | IGHV, EC | p-value |
| OS | 2114.964 | 2096.869 | 0.012 |
| FFS | 4422.823 | 4362.391 | <0.001 |

*Likelihood Ratio Test by comparing the models using −2 log likelihood (−2logL) (Methods)

Focusing on the 49 cases for which there was a discordance between IGHV (heavy chain variable region of immunoglobulin genes) status and EC, it was assessed whether this discordance influenced outcome. failure free survival (FFS) was shorter in discordant M-CLLs and longer in discordant U-CLLs (CLLs with unmutated IGHV) relative to the concordant cases (log-rank test p=0.012 and p=0.0032, respectively) (FIG. 4C). For instance, median failure free survival (FFS) of discordant M-CLLs (i.e., M-CLLs in EC-u clusters) was 3.99 years compared to 6.06 years in concordant cases (M-CLLs in EC-m clusters), thus revealing the added prognostic value of the expression clusters (ECs) relative to traditional classification especially in this subset of CLL.

To systematically assess the features contributing to outcome, IGHV (heavy chain variable region of immunoglobulin genes) subtype, genetic alterations, epitypes, epiCMIT and expression clusters (ECs) were integrated into a multivariable model (FIGS. 4D and 4E, Tables 6A-6C). The n-CLL epitype emerged as one of the strongest predictors of failure free survival (FFS) and OS, emphasizing the known importance of cell of origin. IGHV (heavy chain variable region of immunoglobulin genes) status and epiCMIT also influenced overall survival (OS) to a greater degree than FFS. A relatively limited set of genetic alterations were associated with shorter failure free survival (FFS) (ZNF292, SF3B1, ASXL1, and 17p deletion), but 11 adversely affected overall survival (OS) including novel events such as loss of 5q32. We noted the absence of known alterations, such as ATM and NOTCH1, which were significant by univariate analysis only. This likely reflects co-occurrence with other prognostic factors, similar to an observation with TP53 and 17p deletion. Specific expression clusters (ECs) were particularly informative in the model, with EC-i associated with adverse failure free survival (FFS) and EC-o, EC-m3 and EC-m4 as protective. Altogether, this integrated model reveals a refined prognostic paradigm where genetics, epigenetics, and gene expression classification all contribute to clinical outcome.

TABLE 6A
Integrated analysis assessing impact on overall survival (n = 506)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| ASXL1 | 9 | 1.78 | 4.591 | 2.247 | 9.38 | <.0001 | 2.193 | 0.669783 |
| n-CLL | 211 | 41.7 | 3.129 | 2.262 | 4.33 | <.0001 | 1.595 | 0.50137 |
| loss_8p | 15 | 2.96 | 2.808 | 1.43 | 5.515 | 0.0027 | 2.045 | 0.362337 |
| IGHV_unmutated | 234 | 46.25 | 3.112 | 2.227 | 4.349 | <.0001 | 1.571 | 0.273634 |
| Age >=60 | 317 | 62.65 | 1.613 | 1.149 | 2.265 | 0.0058 | 1.848 | 0.204261 |
| XPO1 | 23 | 4.55 | 2.829 | 1.683 | 4.755 | <.0001 | 1.855 | 0.192345 |
| ZNF292 | 20 | 3.95 | 2.63 | 1.456 | 4.753 | 0.0014 | 2.065 | 0.176254 |
| loss_5q32 | 85 | 16.8 | 2.176 | 1.459 | 3.246 | 0.0001 | 1.366 | 0.175445 |
| SF3B1 | 87 | 17.19 | 2.018 | 1.406 | 2.897 | 0.0001 | 1.297 | 0.142776 |
| ADAMTS4 | 5 | 0.99 | 4.859 | 1.794 | 13.164 | 0.0019 | 3.847 | 0.134184 |
| BIRC3 | 18 | 3.56 | 2.472 | 1.336 | 4.574 | 0.0039 | 1.768 | 0.072579 |
| male | 342 | 67.59 | 1.887 | 1.295 | 2.749 | 0.0009 | 1.431 | 0.052734 |
| gain_8q | 12 | 2.37 | 2.441 | 1.197 | 4.978 | 0.0141 | 1.784 | 0.049247 |
| DYRK1A | 7 | 1.38 | 4.362 | 1.781 | 10.679 | 0.0013 | 2.915 | 0.034476 |
| epiCMIT* | 506 | 100 | x | x | x | x | 1.348 | 0.015027 |
| loss_6q25_3 | 78 | 15.42 | 2.092 | 1.388 | 3.153 | 0.0004 | 1.589 | 0.013 |
| m-CLL | 216 | 42.69 | 0.351 | 0.245 | 0.502 | <.0001 | 0.693 | −0.00459 |
| loss_17p13_3 | 9 | 1.78 | 0.33 | 0.081 | 1.343 | 0.1216 | 0.324 | −0.08221 |
| EC-m4 | 94 | 18.58 | 0.317 | 0.18 | 0.559 | <.0001 | 0.68 | −0.08842 |
| loss_16q22_1 | 6 | 1.19 | 0 | 0 | 0 | 0.9692 | 0 | −0.12646 |
| IKBKB | 4 | 0.79 | 7.148 | 2.268 | 22.529 | 0.0008 | — | — |
| NFKBIE | 7 | 1.38 | 2.6 | 1.061 | 6.369 | 0.0366 | — | — |
| gain_2p | 32 | 6.32 | 1.858 | 1.071 | 3.225 | 0.0276 | — | — |
| loss_11q | 14 | 2.77 | 2.525 | 1.237 | 5.154 | 0.0109 | — | — |
| loss_17p | 23 | 4.55 | 2.464 | 1.332 | 4.558 | 0.0041 | — | — |
| loss_20p | 8 | 1.58 | 4.246 | 1.564 | 11.529 | 0.0045 | — | — |
| loss_11q22_3 | 73 | 14.43 | 1.947 | 1.33 | 2.85 | 0.0006 | — | — |
| loss_10q24_32 | 13 | 2.57 | 2.693 | 1.261 | 5.754 | 0.0105 | — | — |
| loss_15q15_1b | 12 | 2.37 | 2.449 | 1.147 | 5.232 | 0.0207 | — | — |
| loss_1q32_2 | 20 | 3.95 | 2.147 | 1.088 | 4.237 | 0.0276 | — | — |
| loss_1p31_3 | 24 | 4.74 | 2.829 | 1.598 | 5.01 | 0.0004 | — | — |
| ATM | 61 | 12.06 | 1.682 | 1.093 | 2.587 | 0.018 | — | — |
| BRAF | 17 | 3.36 | 1.926 | 1.008 | 3.679 | 0.0472 | — | — |
| CENPB | 1 | 0.2 | 15.931 | 2.173 | 116.821 | 0.0065 | — | — |
| DDX3X | 13 | 2.57 | 3.333 | 1.696 | 6.551 | 0.0005 | — | — |
| GPS2 | 4 | 0.79 | 4.053 | 1.289 | 12.74 | 0.0166 | — | — |
| IKZF3 | 8 | 1.58 | 2.82 | 1.155 | 6.886 | 0.0228 | — | — |
| MGA | 17 | 3.36 | 2.218 | 1.199 | 4.106 | 0.0112 | — | — |
| NOTCH1 | 63 | 12.45 | 1.556 | 1.021 | 2.372 | 0.0395 | — | — |
| EC-u1 | 154 | 30.43 | 2.477 | 1.811 | 3.388 | <.0001 | — | — |
| EC-u2 | 50 | 9.88 | 1.816 | 1.156 | 2.851 | 0.0096 | — | — |
| EC-m3 | 49 | 9.68 | 0.319 | 0.141 | 0.722 | 0.0061 | — | — |

*epiCMIT is a continuous variable
x epiCMIT was not evaluated in univariate analysis since variable is only comparable within epitypes

TABLE 6B
Integrated analysis assessing impact on failure free survival (n = 506)

| | | | | Univariate | Univariate | | | |
|---|---|---|---|---|---|---|---|---|
| | | | Univariate | Lower | Upper | | ENET | |
| | | | Hazard | Confidence | Confidence | Univariate | Hazard | ENET |
| Variable | N | % | Ratio | Limit | Limit | p-value | Ratio | Coefficient |
| n-CLL | 211 | 41.7 | 3.415 | 2.713 | 4.298 | <.0001 | 2.944 | 0.817158 |
| ZNF292 | 20 | 3.95 | 2.917 | 1.806 | 4.711 | <.0001 | 2.786 | 0.442605 |
| EC-i | 52 | 10.28 | 1.63 | 1.177 | 2.258 | 0.0033 | 1.792 | 0.249055 |
| SF3B1 | 87 | 17.19 | 2.027 | 1.562 | 2.631 | <.0001 | 1.449 | 0.234395 |
| loss_17p | 23 | 4.55 | 2.284 | 1.434 | 3.637 | 0.0005 | 1.996 | 0.131223 |
| male | 342 | 67.59 | 1.627 | 1.271 | 2.082 | 0.0001 | 1.309 | 0.109863 |
| ASXL1 | 9 | 1.78 | 3.799 | 1.95 | 7.404 | <.0001 | 1.975 | 0.033793 |
| IGHV_unmutated | 234 | 46.25 | 3.21 | 2.545 | 4.05 | <.0001 | 0.942 | 0.009678 |
| CCND2 | 4 | 0.79 | 0.446 | 0.111 | 1.793 | 0.2555 | 0.357 | −0.03394 |
| EC-m4 | 94 | 18.58 | 0.486 | 0.351 | 0.673 | <.0001 | 0.716 | −0.04251 |
| EC-o | 21 | 4.15 | 0.341 | 0.161 | 0.721 | 0.0049 | 0.444 | −0.10527 |
| m-CLL | 216 | 42.69 | 0.327 | 0.256 | 0.418 | <.0001 | 1.007 | −0.13625 |
| EC-m3 | 49 | 9.68 | 0.263 | 0.151 | 0.458 | <.0001 | 0.36 | −0.29633 |
| GPS2 | 4 | 0.79 | 4.187 | 1.558 | 11.254 | 0.0045 | — | — |
| RSC1A1 | 2 | 0.4 | 6.567 | 1.614 | 26.712 | 0.0086 | — | — |
| TFCP2 | 4 | 0.79 | 7.857 | 2.899 | 21.298 | <.0001 | — | — |
| USP8 | 4 | 0.79 | 5.053 | 1.607 | 15.887 | 0.0056 | — | — |
| ZC3H18 | 5 | 0.99 | 4.147 | 1.702 | 10.108 | 0.0017 | — | — |
| IGLV321_R110 | 45 | 8.89 | 1.607 | 1.129 | 2.286 | 0.0084 | — | — |
| gain_2p | 32 | 6.32 | 2.168 | 1.486 | 3.163 | <.0001 | — | — |
| loss_4p | 8 | 1.58 | 2.434 | 1.203 | 4.924 | 0.0134 | — | — |
| loss_6q | 16 | 3.16 | 1.724 | 1.007 | 2.951 | 0.0471 | — | — |
| gain_8q | 12 | 2.37 | 2.317 | 1.267 | 4.236 | 0.0063 | — | — |
| loss_11q | 14 | 2.77 | 2.265 | 1.298 | 3.953 | 0.004 | — | — |
| tri_12 | 68 | 13.44 | 1.445 | 1.073 | 1.945 | 0.0154 | — | — |
| gain_17q | 5 | 0.99 | 3.145 | 1.297 | 7.624 | 0.0112 | — | — |
| loss_20p | 8 | 1.58 | 4.457 | 2.096 | 9.479 | 0.0001 | — | — |
| gain_19p13_3 | 8 | 1.58 | 2.335 | 1.154 | 4.725 | 0.0183 | — | — |
| loss_11q22_3 | 73 | 14.43 | 2.12 | 1.6 | 2.808 | <.0001 | — | — |
| loss_6q25_3 | 78 | 15.42 | 1.58 | 1.185 | 2.108 | 0.0018 | — | — |
| loss_5q32 | 85 | 16.8 | 1.648 | 1.253 | 2.168 | 0.0004 | — | — |
| loss_2q31_1 | 7 | 1.38 | 2.214 | 1.044 | 4.698 | 0.0383 | — | — |
| loss_7p22_2 | 19 | 3.75 | 1.835 | 1.109 | 3.036 | 0.0182 | — | — |
| loss_12p13_31b | 23 | 4.55 | 1.686 | 1.058 | 2.686 | 0.028 | — | — |
| loss_15q15_1b | 12 | 2.37 | 2.197 | 1.201 | 4.019 | 0.0107 | — | — |
| loss_11q13_4 | 25 | 4.94 | 1.791 | 1.136 | 2.823 | 0.0121 | — | — |
| loss_14q32_12 | 13 | 2.57 | 2.438 | 1.334 | 4.457 | 0.0038 | — | — |
| loss_1p31_3 | 24 | 4.74 | 1.906 | 1.209 | 3.005 | 0.0055 | — | — |
| loss_1p35_2 | 4 | 0.79 | 3.646 | 1.35 | 9.844 | 0.0107 | — | — |
| ATM | 61 | 12.06 | 1.744 | 1.287 | 2.363 | 0.0003 | — | — |
| BCOR | 9 | 1.78 | 3.122 | 1.602 | 6.086 | 0.0008 | — | — |
| BIRC3 | 18 | 3.56 | 2.126 | 1.264 | 3.576 | 0.0045 | — | — |
| BRAF | 17 | 3.36 | 2 | 1.19 | 3.361 | 0.0089 | — | — |
| BRCC3 | 6 | 1.19 | 2.422 | 1.079 | 5.439 | 0.0321 | — | — |
| CARD11 | 8 | 1.58 | 2.116 | 1.001 | 4.477 | 0.0498 | — | — |
| CHKB | 5 | 0.99 | 2.827 | 1.166 | 6.855 | 0.0215 | — | — |
| DDX3X | 13 | 2.57 | 2.267 | 1.24 | 4.144 | 0.0078 | — | — |
| DYRK1A | 7 | 1.38 | 2.38 | 1.059 | 5.349 | 0.0358 | — | — |
| EGR2 | 16 | 3.16 | 1.766 | 1.033 | 3.02 | 0.0376 | — | — |
| FAM50A | 5 | 0.99 | 2.825 | 1.166 | 6.845 | 0.0214 | — | — |
| GNB1 | 4 | 0.79 | 3.404 | 1.261 | 9.188 | 0.0156 | — | — |
| IKBKB | 4 | 0.79 | 3.788 | 1.41 | 10.178 | 0.0083 | — | — |
| IKZF3 | 8 | 1.58 | 2.353 | 1.111 | 4.983 | 0.0255 | — | — |
| IRF4 | 4 | 0.79 | 2.741 | 1.021 | 7.358 | 0.0453 | — | — |
| MYD88 | 16 | 3.16 | 0.406 | 0.167 | 0.982 | 0.0454 | — | — |
| NFKBIE | 7 | 1.38 | 2.811 | 1.251 | 6.316 | 0.0123 | — | — |
| NOTCH1 | 63 | 12.45 | 2.019 | 1.503 | 2.711 | <.0001 | — | — |
| NRAS | 6 | 1.19 | 2.366 | 1.053 | 5.316 | 0.037 | — | — |
| POT1 | 30 | 5.93 | 1.569 | 1.033 | 2.381 | 0.0345 | — | — |
| RFX7 | 6 | 1.19 | 3.142 | 1.399 | 7.056 | 0.0056 | — | — |
| RPS15 | 17 | 3.36 | 1.824 | 1.067 | 3.117 | 0.0281 | — | — |
| SP140 | 2 | 0.4 | 4.538 | 1.126 | 18.297 | 0.0335 | — | — |
| TP53 | 35 | 6.92 | 1.764 | 1.196 | 2.6 | 0.0042 | — | — |
| XPO1 | 23 | 4.55 | 2.064 | 1.337 | 3.188 | 0.0011 | — | — |
| U1 | 21 | 4.15 | 1.705 | 1.072 | 2.713 | 0.0242 | — | — |
| EC-u1 | 154 | 30.43 | 2.484 | 1.977 | 3.122 | <.0001 | — | — |
| EC-u2 | 50 | 9.88 | 1.847 | 1.333 | 2.558 | 0.0002 | — | — |

TABLE 6C
Legend for Tables 6A and 6B

| Column | Description |
|---|---|
| Variable | Feature tested in modeling |
| N | No. patients with feature |
| % | Percent patients with feature |
| Univariate | Hazard Ratio from univariate Cox model |
| Hazard Ratio | |
| Univariate | Hazard Ratio Lower Confidence Limit from univariate |
| Lower | Cox regression model |
| Confidence | |
| Limit | |
| Univariate | Hazard Ratio Upper Confidence Limit from univariate |
| Upper | Cox regression model |
| Confidence | |
| Limit | |
| Univariate p- | p-value from univariate Cox regression model |
| value | |
| ENET | Hazard Ratio in multivariable model (computed by a Cox |
| Hazard Ratio | regression multivariable model for all variables with non- |
| | zero coefficient in the ENET model; see Methods) |
| ENET | Elastic net coefficient ( — indicates 0 coefficient and |
| Coefficient | exclusion from the model) |

Through integration of harmonized multiomic data, the work presented in the above examples has vastly expanded the molecular map of CLL and provided additional insights into its biological and clinical heterogeneity. The number of previously unrecognized putative drivers was doubled, thus achieving a more complete genetic basis for this cancer. These alterations highlight important cellular pathways not previously impacted by candidate drivers that may provide opportunities for development of new therapies in the future. Beyond cataloguing the overall landscape, the distinction between molecular subtypes has been delineated by exploring the extent of variation in the genome, epigenome, and transcriptome. IGHV (heavy chain variable region of immunoglobulin genes) subtypes were enriched in unique genetic driver alterations leading to divergent trajectories of clonal evolution. A significant increase in genetic heterogeneity was found in U-CLL (CLL with unmutated IGHV) with more putative drivers relative to M-CLL. Notably, the driverless samples were almost exclusively M-CLL, suggestive of alternative mechanisms of leukemogenesis in this subtype. Despite this lower genetic complexity, M-CLL (CLL with mutated IGHV) evidently displayed increased transcriptional diversity associated with differences in proliferative history. Furthermore, the discovery of gene expression clusters expanded upon the contemporary disease framework. While specific expression clusters (ECs) were associated with IGHV (heavy chain variable region of immunoglobulin genes) status, epigenetic subtypes, and genetic events, none of these previously defined groups completely captured the diversity exhibited in the expression profiles. Additionally, identifying discordant cases with gene expression profiles inconsistent with their IGHV (heavy chain variable region of immunoglobulin genes) status was prognostic and CHD2 alterations may be contributing to this changed phenotype in M-CLL. This reveals the complex nature of CLL and provides the first version of a comprehensive molecular atlas of CLL that can be the basis for further exploration of unique mechanisms of pathogenesis.

These biological insights were integrated with patient outcomes, which highlighted the prognostic implications of even rare genetic events, such as mutations in ASXL1 and RFX7. Incorporating these data in a unified model revealed the importance of integrating multiple data layers in this disease. Critical components associated with clinical outcome included the cell of origin (IGHV status and epitype), genetic alterations such as 17p deletion, SF3B1 and ZNF292, and gene expression clusters particularly EC-m3 and EC-i. This further refines the current disease paradigm and establishes a new spectrum of events contributing to leukemogenesis that may have implications beyond prognostication. In the future, this molecular foundation may allow for better prediction of response to therapy or provide the basis for rational combination of novel agents.

### Example 6: Drug Sensitivity Correlations

An analysis was completed to determine whether the expression clusters (ECs) can be used to predict the resistance and sensitivity of different chronic lymphocytic leukemias to various drugs. 136 CLL RNA-seqs from Dietrich, et al., “Drug-perturbation-based stratification of blood cancer”, The Journal of Clinical Investigation, 128:427-445 (2018) and their ex-vivo drug sensitivity data was analyzed. The data was retrieved and the machine-learning classifier described herein was applied to each of the 136 CLL RNA-seqs to classify each CLL as belonging to an expression subtype (i.e., Ec-i, EC-m1, EC-m2, EC-m3, EC-m4, EC-o, EC-u1, or EC-u2). The expression subtypes were then used with the drug sensitivity data to compare the percent variability of samples in each expression cluster to the percent viability of samples in all other expression clusters. Statistically significant correlations were found between resistance or sensitivity of a chronic lymphocytic leukemia (CLL) to a particular drug and the CLL's expression subtype (see Tables 7A and 7B). All results presented in Tables 7A and 7B below were statistically significant (i.e., q<0.1) after FDR-correction of the t-test p-values comparing the mean viabilities.

TABLE 7A
Drug sensitivity and resistance

| | | Drug | | Mean | Q (FDR- |
|---|---|---|---|---|---|
| | | response | | viability | corrected |
| | Expression | associated | Mean | in | p-values |
| | cluster | with the | viability in | the other | (Benjamini- |
| Drug name | name | EC | the EC | ECs | Hochberg)) |
| thapsigargin | EC-i | Sensitive | 0.75 | 0.90 | 0.04035519 |
| rotenone | EC-i | Sensitive | 0.63 | 0.77 | 0.05767561 |
| saracatinib | EC-i | Resistant | 1.13 | 1.07 | 0.04352574 |
| PRT062607 | EC-i | Resistant | 0.96 | 0.91 | 0.06140708 |
| HCl | | | | | |
| selumetinib | EC-i | Resistant | 0.97 | 0.92 | 0.09433305 |
| tamatinib | EC-i | Resistant | 1.07 | 1.03 | 0.09664541 |
| BAY 11- | EC-i | Resistant | 1.06 | 1.03 | 0.08300461 |
| 7085 | | | | | |
| dasatinib | EC-m1 | Resistant | 0.90 | 0.73 | 0.03170602 |
| PF 477736 | EC-m1 | Resistant | 1.14 | 0.98 | 0.04208679 |
| AT13387 | EC-m1 | Resistant | 0.82 | 0.72 | 0.09652280 |
| MK-2206 | EC-m1 | Resistant | 1.07 | 1.00 | 0.08270324 |
| SGI-1776 | EC-m1 | Resistant | 1.09 | 1.03 | 0.09577875 |
| TAE684 | EC-m1 | Resistant | 1.07 | 1.01 | 0.04293691 |
| CCT241533 | EC-m1 | Resistant | 1.11 | 1.06 | 0.06222771 |
| fludarabine | EC-m3 | Sensitive | 0.82 | 0.93 | 0.08300461 |
| chaeto- | EC-m3 | Sensitive | 0.98 | 1.07 | 0.05085698 |
| globosin A | | | | | |
| vorinostat | EC-m3 | Sensitive | 1.11 | 1.18 | 0.02031263 |
| afatinib | EC-m3 | Sensitive | 1.03 | 1.08 | 0.04531911 |
| dasatinib | EC-m3 | Resistant | 0.87 | 0.72 | 0.00813509 |
| AZD7762 | EC-m3 | Resistant | 0.95 | 0.82 | 0.00258510 |
| AT13387 | EC-m3 | Resistant | 0.81 | 0.72 | 0.09664541 |
| ibrutinib | EC-m3 | Resistant | 1.05 | 0.96 | 0.00583772 |
| PRT062607 | EC-m3 | Resistant | 1.00 | 0.91 | 0.00089001 |
| HCl | | | | | |
| idelalisib | EC-m3 | Resistant | 1.02 | 0.93 | 0.00008476 |
| duvelisib | EC-m3 | Resistant | 0.95 | 0.88 | 0.00047656 |
| MIS-43 | EC-m3 | Resistant | 0.84 | 0.77 | 0.09577875 |
| PF 477736 | EC-m3 | Resistant | 1.05 | 0.99 | 0.00010631 |
| SD07 | EC-m3 | Resistant | 0.95 | 0.88 | 0.09845663 |
| tamatinib | EC-m3 | Resistant | 1.09 | 1.04 | 0.01477750 |
| selumetinib | EC-m3 | Resistant | 0.97 | 0.93 | 0.02782395 |
| MK-1775 | EC-m3 | Resistant | 1.04 | 1.00 | 0.09577875 |
| KU-60019 | EC-m3 | Resistant | 1.00 | 0.97 | 0.08763103 |
| CCT241533 | EC-m3 | Resistant | 1.09 | 1.06 | 0.04352574 |
| rotenone | EC-m4 | Sensitive | 0.67 | 0.77 | 0.01254118 |
| SNS-032 | EC-m4 | Sensitive | 0.66 | 0.73 | 0.01897653 |
| afatinib | EC-m4 | Sensitive | 1.02 | 1.09 | 0.00115393 |
| dasatinib | EC-m4 | Resistant | 0.89 | 0.70 | 0.00000002 |
| AZD7762 | EC-m4 | Resistant | 0.98 | 0.80 | 0.00000002 |
| PRT062607 | EC-m4 | Resistant | 1.00 | 0.90 | 0.00003184 |
| HCl | | | | | |
| AT13387 | EC-m4 | Resistant | 0.81 | 0.70 | 0.00056500 |
| ibrutinib | EC-m4 | Resistant | 1.04 | 0.95 | 0.00001308 |
| PF 477736 | EC-m4 | Resistant | 1.06 | 0.98 | 0.00293728 |
| duvelisib | EC-m4 | Resistant | 0.94 | 0.87 | 0.00078958 |
| tamatinib | EC-m4 | Resistant | 1.09 | 1.03 | 0.00078958 |
| spebrutinib | EC-m4 | Resistant | 1.01 | 0.95 | 0.01254118 |
| idelalisib | EC-m4 | Resistant | 0.99 | 0.93 | 0.00645942 |
| selumetinib | EC-m4 | Resistant | 0.97 | 0.92 | 0.01477750 |
| MK-2206 | EC-m4 | Resistant | 1.04 | 1.00 | 0.04394774 |
| NU7441 | EC-m4 | Resistant | 1.03 | 0.99 | 0.04293691 |
| TAE684 | EC-m4 | Resistant | 1.04 | 1.01 | 0.04352574 |
| AZD7762 | EC-u1 | Sensitive | 0.74 | 0.88 | 0.00050534 |
| dasatinib | EC-u1 | Sensitive | 0.64 | 0.79 | 0.00024556 |
| PF 477736 | EC-u1 | Sensitive | 0.90 | 1.04 | 0.00000002 |
| YM155 | EC-u1 | Sensitive | 0.53 | 0.64 | 0.04352574 |
| PRT062607 | EC-u1 | Sensitive | 0.86 | 0.95 | 0.00047656 |
| HCl | | | | | |
| ibrutinib | EC-u1 | Sensitive | 0.92 | 0.99 | 0.00247049 |
| AT13387 | EC-u1 | Sensitive | 0.68 | 0.75 | 0.06116290 |
| tamatinib | EC-u1 | Sensitive | 1.00 | 1.06 | 0.000 |
| idelalisib | EC-u1 | Sensitive | 0.90 | 0.96 | 0.017 |
| duvelisib | EC-u1 | Sensitive | 0.85 | 0.90 | 0.024 |
| KX2-391 | EC-u1 | Sensitive | 0.86 | 0.91 | 0.016 |
| spebrutinib | EC-u1 | Sensitive | 0.92 | 0.98 | 0.044 |
| TAE684 | EC-u1 | Sensitive | 0.98 | 1.03 | 0.022 |
| MK-2206 | EC-u1 | Sensitive | 0.98 | 1.02 | 0.040 |
| MK-1775 | EC-u1 | Sensitive | 0.98 | 1.02 | 0.004 |
| SGI-1776 | EC-u1 | Sensitive | 1.01 | 1.04 | 0.001 |
| selumetinib | EC-u1 | Sensitive | 0.91 | 0.94 | 0.097 |
| BAY 11- | EC-u1 | Sensitive | 1.02 | 1.05 | 0.083 |
| 7085 | | | | | |
| BX912 | EC-u1 | Sensitive | 1.05 | 1.07 | 0.056 |
| actinomycin | EC-u1 | Resistant | 0.37 | 0.27 | 0.001 |
| D | | | | | |
| venetoclax | EC-u1 | Resistant | 0.54 | 0.45 | 0.024 |
| navitoclax | EC-u1 | Resistant | 0.89 | 0.82 | 0.014 |
| everolimus | EC-u1 | Resistant | 0.99 | 0.95 | 0.044 |
| AZD7762 | EC-u2 | Sensitive | 0.51 | 0.85 | 0.000 |
| dasatinib | EC-u2 | Sensitive | 0.46 | 0.75 | 0.000 |
| AT13387 | EC-u2 | Sensitive | 0.50 | 0.74 | 0.000 |
| ibrutinib | EC-u2 | Sensitive | 0.80 | 0.98 | 0.052 |
| duvelisib | EC-u2 | Sensitive | 0.73 | 0.89 | 0.019 |
| idelalisib | EC-u2 | Sensitive | 0.79 | 0.95 | 0.044 |
| selumetinib | EC-u2 | Sensitive | 0.79 | 0.94 | 0.044 |
| PRT062607 | EC-u2 | Sensitive | 0.78 | 0.93 | 0.037 |
| HCl | | | | | |
| cephaeline | EC-u2 | Sensitive | 0.72 | 0.82 | 0.095 |
| spebrutinib | EC-u2 | Sensitive | 0.88 | 0.96 | 0.060 |
| NU7441 | EC-u2 | Sensitive | 0.92 | 1.00 | 0.048 |
| KU-60019 | EC-u2 | Sensitive | 0.90 | 0.97 | 0.083 |
| rotenone | EC-u2 | Resistant | 0.92 | 0.74 | 0.00 |
| thapsigargin | EC-u2 | Resistant | 1.00 | 0.87 | 0.09 |

TABLE 7B
Drug targets

| | | | | Pathway |
|---|---|---|---|---|
| Drug | | Drug target | Drug target | targeted by |
| name | Main drug targets | category | group | drug |
| thapsigargin | Sarco/endoplasmic reticulum | Other | other | Other |
| | Ca2+ ATPase (SERCA) | | | |
| rotenone | Electron transport chain in | Mitochondrial | other | Other |
| | mitochondria | metabolism | | |
| saracatinib | SRC, ABL1 | B-cell receptor | kinase | Other |
| | | | inhibitor | |
| PRT062607 | SYK | B-cell receptor | kinase | B-cell |
| HCl | | | inhibitor | receptor |
| selumetinib | MEK1/2 | MAPK | kinase | Other |
| | | | inhibitor | |
| tamatinib | SYK | B-cell receptor | kinase | B-cell |
| | | | inhibitor | receptor |
| BAY 11-7085 | NFkB | NFkB | other | Other |
| dasatinib | ABL1, KIT, LYN, PDGFRA, | BCR/ABL | kinase | Other |
| | PDGFRB, SRC | | inhibitor | |
| PF | CHK1, CHK2 | DNA damage | kinase | Other |
| 477736 | | response | inhibitor | |
| AT13387 | HSP90 | HSP90 | other | Other |
| MK-2206 | AKT1/2 (PKB) | PI3K/AKT | kinase | Other |
| | | | inhibitor | |
| SGI-1776 | PIM | PIM | kinase | Other |
| | | | inhibitor | |
| TAE684 | ALK | ALK | other | Other |
| CCT241533 | CHK2 | DNA damage | kinase | Other |
| | | response | inhibitor | |
| fludarabine | Purine analogue | DNA damage | chemo- | Other |
| | | response | therapeutic | |
| | | | agent | |
| chaetoglobosin | Actin | Cytoskeleton | other | Other |
| A | | | | |
| vorinostat | HDAC I/IIa/IIb/IV | Epigenome | other | Other |
| afatinib | EGFR, ERBB2 | EGFR | kinase | Other |
| | | | inhibitor | |
| dasatinib | ABL1, KIT, LYN, PDGFRA, | BCR/ABL | kinase | Other |
| | PDGFRB, SRC | | inhibitor | |
| AZD7762 | CHK1/2 | DNA damage | kinase | Other |
| | | response | inhibitor | |
| AT13387 | HSP90 | HSP90 | other | Other |
| ibrutinib | BTK | B-cell receptor | kinase | B-cell |
| | | | inhibitor | receptor |
| PRT062607 | SYK | B-cell receptor | kinase | B-cell |
| HCl | | | inhibitor | receptor |
| idelalisib | PI3K delta | PI3K/AKT | kinase | B-cell |
| | | | inhibitor | receptor |
| duvelisib | PI3K gamma, PI3K delta | PI3K/AKT | kinase | B-cell |
| | | | inhibitor | receptor |
| MIS-43 | ROS | Reactive | other | Reactive |
| | | oxygen | | oxygen |
| | | species | | species |
| PF | CHK1, CHK2 | DNA damage | kinase | Other |
| 477736 | | response | inhibitor | |
| SD07 | ROS | Reactive | other | Reactive |
| | | oxygen | | oxygen |
| | | species | | species |
| tamatinib | SYK | B-cell receptor | kinase | B-cell |
| | | | inhibitor | receptor |
| selumetinib | MEK1/2 | MAPK | kinase | Other |
| | | | inhibitor | |
| MK-1775 | WEE1 | Cell cycle | kinase | Other |
| | | control | inhibitor | |
| KU- | ATM | DNA damage | kinase | Other |
| 60019 | | response | inhibitor | |
| CCT241533 | CHK2 | DNA damage | kinase | Other |
| | | response | inhibitor | |
| rotenone | Electron transport chain in | Mitochondrial | other | Other |
| | mitochondria | metabolism | | |
| SNS-032 | CDK2/7/9 | Cell cycle | kinase | Other |
| | | control | inhibitor | |
| afatinib | EGFR, ERBB2 | EGFR | kinase | Other |
| | | | inhibitor | |
| dasatinib | ABL1, KIT, LYN, PDGFRA, | BCR/ABL | kinase | Other |
| | PDGFRB, SRC | | inhibitor | |
| AZD7762 | CHK1/2 | DNA damage | kinase | Other |
| | | response | inhibitor | |
| PRT062607 | SYK | B-cell receptor | kinase | B-cell |
| HCl | | | inhibitor | receptor |
| AT13387 | HSP90 | HSP90 | other | Other |
| ibrutinib | BTK | B-cell receptor | kinase | B-cell |
| | | | inhibitor | receptor |
| PF | CHK1, CHK2 | DNA damage | kinase | Other |
| 477736 | | response | inhibitor | |
| duvelisib | PI3K gamma, PI3K delta | PI3K/AKT | kinase | B-cell |
| | | | inhibitor | receptor |
| tamatinib | SYK | B-cell receptor | kinase | B-cell |
| | | | inhibitor | receptor |
| spebrutinib | BTK | B-cell receptor | kinase | B-cell |
| | | | inhibitor | receptor |
| idelalisib | PI3K delta | PI3K/AKT | kinase | B-cell |
| | | | inhibitor | receptor |
| selumetinib | MEK1/2 | MAPK | kinase | Other |
| | | | inhibitor | |
| MK-2206 | AKT1/2 (PKB) | PI3K/AKT | kinase | Other |
| | | | inhibitor | |
| NU7441 | DNAPK | DNA damage | kinase | Other |
| | | response | inhibitor | |
| TAE684 | ALK | ALK | other | Other |
| AZD7762 | CHK1/2 | DNA damage | kinase | Other |
| | | response | inhibitor | |
| dasatinib | ABL1, KIT, LYN, PDGFRA, | BCR/ABL | kinase | Other |
| | PDGFRB, SRC | | inhibitor | |
| PF | CHK1, CHK2 | DNA damage | kinase | Other |
| 477736 | | response | inhibitor | |
| YM155 | Survivin | Apoptosis | other | Other |
| | | (BH3, | | |
| | | Survivin) | | |
| PRT062607 | SYK | B-cell receptor | kinase | B-cell |
| HCl | | | inhibitor | receptor |
| ibrutinib | BTK | B-cell receptor | kinase | B-cell |
| | | | inhibitor | receptor |
| AT13387 | HSP90 | HSP90 | other | Other |
| tamatinib | SYK | B-cell receptor | kinase | B-cell |
| | | | inhibitor | receptor |
| idelalisib | PI3K delta | PI3K/AKT | kinase | B-cell |
| | | | inhibitor | receptor |
| duvelisib | PI3K gamma, PI3K delta | PI3K/AKT | kinase | B-cell |
| | | | inhibitor | receptor |
| KX2-391 | SRC | B-cell receptor | kinase | Other |
| | | | inhibitor | |
| spebrutinib | BTK | B-cell receptor | kinase | B-cell |
| | | | inhibitor | receptor |
| TAE684 | ALK | ALK | other | Other |
| MK-2206 | AKT1/2 (PKB) | PI3K/AKT | kinase | Other |
| | | | inhibitor | |
| MK-1775 | WEE1 | Cell cycle | kinase | Other |
| | | control | inhibitor | |
| SGI-1776 | PIM | PIM | kinase | Other |
| | | | inhibitor | |
| selumetinib | MEK1/2 | MAPK | kinase | Other |
| | | | inhibitor | |
| BAY 11- | NFkB | NFkB | other | Other |
| 7085 | | | | |
| BX912 | PDK1 | PI3K/AKT | kinase | Other |
| | | | inhibitor | |
| actinomycin | RNA synthesis | DNA damage | other | Other |
| D | | response | | |
| venetoclax | BCL2 | Apoptosis | other | Apoptosis |
| | | (BH3, | | (BH3) |
| | | Survivin) | | |
| navitoclax | BCL2, BCL-XL, BCL-W | Apoptosis | other | Apoptosis |
| | | (BH3, | | (BH3) |
| | | Survivin) | | |
| everolimus | mTOR | mTOR | other | Other |
| AZD7762 | CHK1/2 | DNA damage | kinase | Other |
| | | response | inhibitor | |
| dasatinib | ABL1, KIT, LYN, PDGFRA, | BCR/ABL | kinase | Other |
| | PDGFRB, SRC | | inhibitor | |
| AT13387 | HSP90 | HSP90 | other | Other |
| ibrutinib | BTK | B-cell receptor | kinase | B-cell |
| | | | inhibitor | receptor |
| duvelisib | PI3K gamma, PI3K delta | PI3K/AKT | kinase | B-cell |
| | | | inhibitor | receptor |
| idelalisib | PI3K delta | PI3K/AKT | kinase | B-cell |
| | | | inhibitor | receptor |
| selumetinib | MEK1/2 | MAPK | kinase | Other |
| | | | inhibitor | |
| PRT062607 | SYK | B-cell receptor | kinase | B-cell |
| HCl | | | inhibitor | receptor |
| cephaeline | 40S ribosomal subunit | Other | other | Other |
| spebrutinib | BTK | B-cell receptor | kinase | B-cell |
| | | | inhibitor | receptor |
| NU7441 | DNAPK | DNA damage | kinase | Other |
| | | response | inhibitor | |
| KU- | ATM | DNA damage | kinase | Other |
| 60019 | | response | inhibitor | |
| rotenone | Electron transport chain in | Mitochondrial | other | Other |
| | mitochondria | metabolism | | |
| thapsigargin | Sarco/endoplasmic reticulum | Other | other | Other |
| | Ca2+ ATPase (SERCA) | | | |

The following methods and materials were employed in Examples 1-5.

### Data Availability

Sequencing, expression, and genotyping is available at European Genome-Phenome Archive (EGA), which is hosted at the European Bioinformatics Institute (EBI), under accession numbers EGAS00000000092 and in dbGaP under accession numbers: phs001473, phs000922.v2.p1, phs001431, phs001091.v1.01, phs000435.v3.p1, phs002297.v1, phs000879.v1.p1. 450k array data is available at EGA under accession number EGAD00010001975.

### Code Availability

Terra methods can be found at app.terra.bio/. The new epiCMIT suitable for Illumina arrays and NGS approaches can be found at github.com. The RFcaller pipeline is available at github.com. Additional code used for the project can be found at github.com.

### Human Samples

The 1156 CLL/MBL samples (1010 CLL samples were used in the clinical analysis) included tumor and germline samples collected either during active surveillance (n=687), post-treatment (n=52), or at enrollment of a clinical trial prior to first cycle of therapy (n=417; treatment-naive n=371, relapsed/refractory n=46). Briefly, these trials included: (i) comparison of fludarabine and cyclophosphamide (FC) to FC-rituximab (FCR) in previously untreated patients (CLL8 trial, n=309); (ii) treatment-naive TP53 mutated patients within phase 2 CLL20 trial who all received alemtuzumab (n=31); (iii) ibrutinib or R-ibrutinib in relapsed/refractory (R/R) or untreated patients with 17p deletion, TP53 mutation, and/or 11q deletion (n=77; treatment-naive n=31; R/R n=46). If multiple samples were obtained from a patient, then the earliest collected sample was selected for analysis. Peripheral blood mononuclear cells were isolated and DNA and/or RNA were extracted and prepared as previously described (Stilgenbauer, S. et al. Gene mutations and treatment outcome in chronic lymphocytic leukemia: results from the CLL8 trial. Blood 123, 3247-3254 (2014). 2. Landau, D. A. et al. Mutations driving CLL and their evolution in progression and relapse. Nature 526, 525 (2015); Puente, X. S. et al. Non-coding recurrent mutations in chronic lymphocytic leukaemia. Nature 526, 519 (2015); Gruber, M. et al. Growth dynamics in naturally progressing chronic lymphocytic leukaemia. Nature 570, 474-479 (2019); Landau, D. A. et al. The evolutionary landscape of chronic lymphocytic leukemia treated with ibrutinib targeted therapy. Nat. Commun. 8, 2185 (2017); Kasar, S. et al. Whole-genome sequencing reveals activation-induced cytidine deaminase signatures during indolent chronic lymphocytic leukaemia evolution. Nat. Commun. 6, 8866 (2015); Burger, J. A. et al. Safety and activity of ibrutinib plus rituximab for patients with high-risk chronic lymphocytic leukaemia: a single-arm, phase 2 study. Lancet Oncol. 15, 1090-1099 (2014); Burger, J. A. et al. Clonal evolution in patients with chronic lymphocytic leukaemia developing resistance to BTK inhibition. Nat. Commun. 7, 11589 (2016)).

### Molecular Data Retrieval and Assembly

Previously reported sequencing data was retrieved from CLL and MBL samples, including 984 whole-exome sequences, 177 whole-genome sequences, 453 RNA-seqs, 490 methylation 450k arrays, and 547 reduced-representation bisulfite sequencing. Additionally, 264 RNA-seq samples were sequenced, and targeted DNA sequencing of the NOTCH1 3′ UTR was performed for 293 samples, as described below.

### RNA-Seq Generation

For cDNA Library Construction, total RNA was quantified using the Quant-iT RiboGreen RNA Assay Kit and normalized to 5 ng/ul. Following plating, 2 uL of ERCC controls (using a 1:1000 dilution) were spiked into each sample. An aliquot of 200 ng for each sample underwent library preparation using an automated variant of the Illumina TruSeq Stranded mRNA Sample Preparation Kit, followed by heat fragmentation and cDNA synthesis from the RNA template. The resultant 400 bp cDNA then underwent dual-indexed library preparation, consisting of ‘A’ base addition, adapter ligation using P7 adapters, and PCR enrichment using P5 adapters. After enrichment, the libraries were quantified using Quant-iT PicoGreen (1:200 dilution). After normalizing samples to 5 ng/uL, the set was pooled and quantified using the KAPA Library Quantification Kit for Illumina Sequencing Platforms.

For Illumina sequencing, pooled libraries were normalized to 2 nM and denatured using 0.1 N NaOH prior to sequencing. Flowcell cluster amplification and sequencing were performed according to the manufacturer's protocols using the NovaSeq 6000, HiSeq 2000 or HiSeq 2500. Each run was a 101 bp paired-end read with eight-base index barcodes. Raw data was analyzed using the Broad Picard Pipeline which includes de-multiplexing and data aggregation.

### Sequence Data Processing and Analysis

All sequencing data (WES, WGS, RNA-seq, RRBS and targeted NOTCH1 sequencing) were processed and analyzed using methods implemented in the Broad Institute's cloud-based Terra platform (app.terra.bio).

### WES/WGS Alignment and Quality Control

All DNA sequence data was processed through the Broad Institute's data processing pipeline. For each sample, this pipeline combines data from multiple libraries and flow cell runs into a single BAM file. This file contains reads aligned to the human genome hg19 genome assembly (version b37) done by the Picard and Genome Analysis Toolkit (GATK) developed at the Broad Institute, a process that involves marking duplicate reads, recalibrating base qualities and realigning around indels. Reads were aligned to the hg19 genome assembly (version b37) using BWA-MEM (version 0.7.15-r1140).

### Mutation Calling

Prior to variant calling, the impact of oxidative damage (oxoG) to DNA during sequencing was quantified using DeToxoG (Costello, M. et al. Discovery and characterization of artifactual mutations in deep coverage targeted capture sequencing data due to oxidative DNA damage during sample preparation. Nucleic Acids Res. 41, e67 (2013)). The cross-sample contamination was measured with ContEst based on the allele fraction of homozygous SNPs (Cibulskis, K. et al. ContEst: estimating cross-contamination of human samples in next-generation sequencing data. Bioinformatics 27, 2601-2602 (2011)), and this measurement was used in the downstream mutation calling pipeline. From the aligned BAM files, somatic alterations were identified using a set of tools developed at the Broad Institute (broadinstitute.org/cancer/cga). The details of the sequencing data processing have been described elsewhere (Berger, M. F. et al. The genomic complexity of primary human prostate cancer. Nature 470, 214-220 (2011); Chapman, M. A. et al. Initial genome sequencing and analysis of multiple myeloma. Nature 471, 467-472 (2011)). Briefly, for sSNVs/indel detection, high-confidence somatic mutation calls were made by applying MuTect (Cibulskis, K. et al. Sensitive detection of somatic point mutations in impure and heterogeneous cancer samples. Nat. Biotechnol. 31, 213-219 (2013)), MuTect2 (Benjamin, D. et al. Calling Somatic SNVs and Indels with Mutect2. bioRxiv 861054 (2019) doi:10.1101/861054) and Strelka2 (Kim, S. et al. Strelka2: fast and accurate calling of germline and somatic variants. Nat. Methods 15, 591-594 (2018)) to WES/WGS sequencing data. Given that normal blood samples might also contain CLL cells, DeTiN (Taylor-Weiner, A. et al. DeTiN: overcoming tumor-in-normal contamination. Nat. Methods 15, 531-534 (2018)) was used to estimate tumor in normal (TiN) contamination in order to recover falsely rejected sSNVs/indels. Next, four types of filters were applied: (i) a realignment-based filter, which removes variants that can be attributed entirely to ambiguously mapped reads; (ii) an orientation bias filter, which removes possible oxoG and FFPE artifacts; (iii) a ContEst filter, which removes variants that might come from other samples due to contamination; and (iv) an allele fraction specific panel-of-normals filter, which compares the detected variants to a large panel of normal exomes or genomes and removes variants that were observed in the two panel-of-normals (PoNs): one consists of 8,334 normal samples in TCGA while the other consists of 481 CLL-matched normal samples with TiN estimates of 0. All four filters together contributed to the exclusion of potential false-positive events (e.g. commonly occurring germline variants or sequencing artifacts), which ultimately yielded the final list of mutations. All filtered events in candidate CLL driver genes were also manually reviewed using the Integrated Genomics Viewer (IGV) (Robinson, J. T. et al. Integrative genomics viewer. Nat. Biotechnol. 29, 24-26 (2011)).

In order to increase the sensitivity and precision of mutation calls in candidate driver genes, an additional variant calling step was performed for the candidate driver gene loci using Rfcaller (github.com/xa-lab/RFcaller), a pipeline that uses read-level features and extra trees/random forest algorithms for the detection of somatic mutations. This pipeline was run with default parameters for whole exome sequencing (WES) or whole genome sequencing (WGS) data, as well as for RNA-seq data for NOTCH1, which has low coverage in hotspot regions in some samples due to high GC content. All candidate mutations that passed filters and were detected by both pipelines were considered positives. Mutations detected by only one of the callers were visually inspected by a set of at least four expert curators, considering the following exclusion criteria: (i) low evidence due to limited number of reads supporting the mutation in the tumor sample or excessive mutant reads in the normal sample; (ii) low depth of coverage to rule out germline variant; (iii) low base quality region; (iv) low mapping quality region leading to multi-mapped reads; (v) calls supported by reads with a strong strand bias.

### Identification of Significantly Mutated Genes

To identify candidate cancer genes using the mutation calls from WES, SignatureAnalyzer (Kim, J. et al. Somatic ERCC2 mutations are associated with a distinct genomic signature in urothelial tumors. Nat. Genet. 48, 600-606 (2016)) was first used to identify mutational processes and potential artifact signatures. A signature likely due to the bleedthrough sequencing artifact was discovered and then mutations with greater than 95% chance attributed to that bleedthrough signature were filtered. Next, MutSig2CV (Lawrence, M. S. et al. Mutational heterogeneity in cancer and the search for new cancer-associated genes. Nature 499, 214-218 (2013)) was run to identify driver genes from the filtered whole exome sequencing (WES) Mutation Annotation Format (MAF) file. A stringent manual review was conducted using the IGV (Robinson, J. T. et al. Integrative genomics viewer. Nat. Biotechnol. 29, 24-26 (2011)) to review the mutations in the driver genes and further exclude low evidence calls. Then MutSig2CV was rerun on the filtered set of mutation calls from whole exome sequencing (WES) to identify the final candidate driver genes. In addition, CLUMPS (Kamburov, A. et al. Comprehensive assessment of cancer missense mutation clustering in protein structures. Proc. Natl. Acad. Sci. U.S.A 112, E5486-95 (2015)) was used to identify driver genes based on clustering of mutations in the 3D structure of the protein product. For CLUMPS, two FDR corrections were applied: one for all candidates and a second restricted hypothesis testing focused on genes in the COSMIC Cancer Gene Census (Sondka, Z. et al. The COSMIC Cancer Gene Census: describing genetic dysfunction across all human cancers. Nat. Rev. Cancer 18, 696-705 (2018)). Finally, for further stringency and to exclude candidates irrelevant to CLL biology, candidate genes that were not expressed in RNA-seq of 610 treatment-naive CLL samples were discarded using a one-sided t-test testing for difference from 0 in transcripts per million (TPM) space. This discarded 15 candidate genes.

### U1 g.3A>C Mutational Status

The U1 g.3A>C mutational status for 294 cases from the ICGC cohort was previously reported (Shuai, S. et al. The U1 spliceosomal RNA is recurrently mutated in multiple cancers. Nature 574, 712-716 (2019)). For the remaining 212 ICGC cases, U1 status was determined using a previously validated rhAMP SNP assay (Integrated DNA Technology) (Shuai, S. et al.). The U1 status of 425 patients from the DFCI/Broad cohort was inferred from RNA-seq data using a random forest classifier with 100 trees built from 3,174 differentially spliced introns between U1 mutated and wild-type cases, as previously described (Shuai, et al.). A cohort of 104 cases from the ICGC cohort (7 mutated, 97 wild-type) was used to train the model, while 54 cases (3 mutated, 51 wild-type) were used as a test (Shuai, et al.). Altogether, the U1 g.3A>C status was determined for 931 of 1156 cases.

### NOTCH1 Mutation Calling

A subset of the whole exome sequencing (WES) data had reduced coverage in the GC-rich region of NOTCH1, a common and clinically-relevant driver in CLL. The NOTCH1 calls from WES/WGS were augmented by Sanger sequencing, targeted deep sequencing of NOTCH1 3′ UTR (details below), and manual review of all WES, whole genome sequencing (WGS) and RNA-seq in IGV (Robinson, J. T. et al. Integrative genomics viewer. Nat. Biotechnol. 29, 24-26 (2011)). This was primarily focused on identifying NOTCH1 hotspot CT deletion p.P2515Rfs*4 and NOTCH1 3′ UTR mutational hotspot chr9:139390152T>C. RNA-seq review was based on the direct mutation and the splicing perturbation associated with the 3′ UTR mutation.

### Targeted Sequencing of NOTCH1 3′ UTR

To amplify the region of the NOTCH1 3′ UTR hotspot mutation at position chr9:139390152T>C and adjacent sequence from genomic DNA, the following PCR1 reaction mix was prepared including 1×PfX amplification buffer, 1×PfX enhancer solution (ThermoFisher, 11708039), 0.3 mM each dNTPs, 1 mM MgSO₄, 0.6 μM of NOTCH/1^{st} F-primer, 0.6 μM of Notch1 1^{st} R-primer. To each well of a 96 well plate, 46 μL of this mix was added and 2 μL of DNA sample (25 ng/μL concentration), and then following PCR reaction was performed: 95° C. 5 min, 33 cycles of (95° C. 30 s, 55° C. 30 S, 68° C. 1 min), and then held at 4° C. Once the plate heated to 95° C. for 1 min, the reaction was paused, and the plate was taken out and 2 μL Pfx polymerase mix (1:4 diluted Pfx Polymerase with water) was added into each well, and then reaction program was continued. In order to add an identifier index onto each amplicon, the PCR2 was performed. First, the following reaction mix was prepared containing 1×Kapa HiFi Fidelity buffer (2 mM MgCl₂), 0.41 mM of each dNTPs, 1 μL of Kapa HiFi hotstart polymerase (KapaBiosystems, KK2101), 0.82 μM of the forward primer, and 0.82 μM of each reverse primer (in a 96 well plate). Then 50 μL of the mix was added to a new 96 well plate and 10 μL of the PCR1 mix was added to each well of the plate, and the following PCR reaction was performed: 98° C. 45 s, 8 cycles of (98° C. 15 s, 60° C. 30 s, 72° C. 30 s), 72° C. 1 min and then held at 4° C. After PCR2, 3 μL of each of the indexed PCR products was pooled and cleaned up using Ampure XP beads. After cleaning, the pooled libraries were quantified using a Bioanalyzer, and then sequenced on a MiSeq using the following parameters: Read 1: 200 nt, Read 2: 100nt, Index 1: 8nt, and index 2: 8nt.

### Copy Number Analysis

For detecting somatic copy number alterations (sCNAs) the GATK4 CNV pipeline (github.com/gatk-workflows/gatk4-somatic-cnvs) was used, which involves the CalculateTargetCoverage, NormalizeSomaticReadCounts, and Circular Binary Segmentation (CBS) algorithms (Olshen, A. B., Venkatraman, E. S., Lucito, R. & Wigler, M. Circular binary segmentation for the analysis of array-based DNA copy number data. Biostatistics 5, 557-572 (2004)) for genome segmentation. In order to identify candidate somatic copy number alteration (sCNA) drivers (genomic regions that are significantly amplified or deleted), GISTIC 2.0 was then applied (Mermel, C. H. et al. GISTIC2.0 facilitates sensitive and confident localization of the targets of focal somatic copy-number alteration in human cancers. Genome Biol. 12, R41 (2011)). To exclude potential germline CNAs, GISTIC 2.0 was first run on the matched normal samples and then the recurrent CNAs this outputted (q<0.1) was concatenated to the blacklisted regions. Then GISTIC 2.0 was run on the tumor samples to produce a list of candidate somatic copy number alteration (sCNA) driver regions. A force-calling process was applied to identify the presence/absence of each somatic copy number alteration (sCNA) driver event across tumor samples (github.com/getzlab/GISTIC2_postprocessing). To further filter the potential false positive drivers, only somatic copy number alteration (sCNA) drivers with population frequency greater than 1% were accepted. Finally, all filtered somatic copy number alteration (sCNA) drivers were manually reviewed using IGV (Robinson, J. T. et al. Integrative genomics viewer. Nat. Biotechnol. 29, 24-26 (2011)) to exclude drivers that are based on somatic copy number alteration (sCNA) events with low supporting evidence or that were localized close to centromeres. somatic copy number alteration (sCNA) drivers were annotated by intersection with our list of CLL mutation driver genes and with genes in the COSMIC Cancer Gene Census (Sondka, Z. et al. The COSMIC Cancer Gene Census: describing genetic dysfunction across all human cancers. Nat. Rev. Cancer 18, 696-705 (2018)) (v90).

### Structural Variants Calling

For structural variation (SV) detection, the pipeline integrated evidence from three structural variation detection algorithms (Manta (Chen, X. et al. Manta: rapid detection of structural variants and indels for germline and cancer sequencing applications. Bioinformatics 32, 1220-1222 (2016)), SvABA (Wala, J. A. et al. SvABA: genome-wide detection of structural variants and indels by local assembly. Genome Res. 28, 581-591 (2018)) and dRanger (Berger, M. F. et al. The genomic complexity of primary human prostate cancer. Nature 470, 214-220 (2011); Bass, A. J. et al. Genomic sequencing of colorectal adenocarcinomas identifies a recurrent VTI1A-TCF7L2 fusion. Nat. Genet. 43, 964-968 (2011); Chapman, M. A. et al. Initial genome sequencing and analysis of multiple myeloma. Nature 471, 467-472 (2011)) to generate a list of structural variation events with high confidence. The three SV detection tools were followed with BreakPointer (Drier, Y. et al. Somatic rearrangements across cancer reveal classes of samples with distinct patterns of DNA breakage and rearrangement-induced hypermutability. Genome Res. 23, 228-235 (2013)) to pinpoint the exact breakpoint at base-level resolution. Breakpoint information was aggregated per sample to identify: (i) balanced translocations, which were defined as those with breakpoints on reverse strands within 1-kb of each other; (ii) inversions supported on both ends; (iii) complex events, based on the number of clustered events within 50-kb of each other. Breakpoints were annotated by intersection with the lists of CLL driver genes and significant somatic copy number alteration (sCNA) regions, as well as with genes in the COSMIC Cancer Gene Census (v90) (Sondka, Z. et al. The COSMIC Cancer Gene Census: describing genetic dysfunction across all human cancers. Nat. Rev. Cancer 18, 696-705 (2018)).

### Identification of Structural Variants Involving the Immunoglobulin (IG) Loci

Potentially oncogenic structural variants involving any of the IG loci were analyzed using IgCaller (v1.1) (Nadeu, F. et al. IgCaller for reconstructing immunoglobulin gene rearrangements and oncogenic translocations from whole-genome sequencing in lymphoid neoplasms. Nat. Commun. 11, 3390 (2020)) and visually inspected in IGV (Robinson, J. T. et al. Integrative genomics viewer. Nat. Biotechnol. 29, 24-26 (2011)). The breakpoints of the IG loci were used to determine the underlying mechanisms leading to these events. To that end, a search was done for evidence of aberrant V(D)J recombination (i.e., breakpoints in any of the V(D)J genes and close to recombination-activation gene (RAG) signal sequences) or aberrant class switch recombination (CSR) (i.e., breakpoints located in any of the CSR regions). IG genes and CSR regions were annotated based on the annotations used by IgCaller. Of note, no evidence of IG structural variants mediated by somatic hypermutation (SHM) were identified (i.e., events with breakpoints within already rearranged V(D)J genes linked with the presence of SHM).

### Estimation of Purity, Ploidy, and Cancer Cell Fraction (CCF)

To estimate sample purity, ploidy, absolute allele-specific copy number and cancer cell fraction (CCF) of the filtered whole exome sequencing (WES) somatic coding mutations, ABSOLUTE (Carter, S. L. et al. Absolute quantification of somatic DNA alterations in human cancer. Nat. Biotechnol. 30, 413-421 (2012)) was used, which integrates allele fraction specific information from the sequencing data for sSNVs/indels and sCNAs. For each sample, manual review was conducted to determine the optimal ABSOLUTE solution. Using these ABSOLUTE solutions allowed for recovery of CCF estimates for 49,882 coding mutations of all 53,489 mutations (93.3%) identified in whole exome sequencing (WES) data.

### Timing Analysis

To infer phylogenetic and evolutionary trajectories based on somatic mutations and copy number variation, PhylogicNDT Cluster, Timing, LeagueModel modules were applied (Leshchiner, I. et al. Comprehensive analysis of tumour initiation, spatial and temporal progression under multiple lines of treatment. bioRxiv 508127 (2019)) (github: github.com/broadinstitute/PhylogicNDT) on the filtered whole exome sequencing (WES) MAF with CCF annotated from the optimal ABSOLUTE solution. To determine if shared events had significantly different order of acquisition in M-CLL (CLL with mutated IGHV) and U-CLL, the timing score was randomly sampled 250,000 times for each shared event from the MCMC traces of M-CLL (CLL with mutated IGHV) and U-CLL (CLL with unmutated IGHV) respectively, and the difference between the two scores was calculated. Then the frequency of the differences being less than 0 was calculated. If the frequency was less than 0.5, then the p-value was assigned as two times the frequency to that event, i.e. p-value=2*freq; else, the p-value was assigned as two times one minus the frequency to that event, i.e. p-value=2*(1−freq). Then the Benjamini-Hochberg multiple hypothesis correction procedure was applied to all the p-values of shared driver events. The timing of a shared driver event was considered significantly different between the two subtypes if the corresponding q value was less than 0.1.

### Gene Set Enrichment for Driver Genes

Gene set enrichment analysis was performed using g:profiler (Reimand, J. et al. g:Profiler-a web server for functional interpretation of gene lists (2016 update). Nucleic Acids Res. 44, W83-9 (2016)) on the 97 driver genes, the total identified in the MutSig and CLUMPS analyses for “All,” M-CLL, and U-CLL (CLL with unmutated IGHV) (excluding genes detected only by CLUMPS restricted hypothesis testing for cancer genes, n=2; and excluding 5 genes not found in the gene set annotation). Gene sets from MSigDB v7.0 were used, aggregating Hallmark, C5:GO:BP and C2:CP:REACTOME collections. g:profiler results were filtered by q<0.1, restricted in size between 5 and 350 genes in the gene set, and required to include at least two drivers. To identify similar biological processes and remove redundancy in overlapping gene sets, significant gene sets were clustered using Louvain clustering (Blondel, V. D., Guillaume, J.-L., Lambiotte, R. & Lefebvre, E. Fast unfolding of communities in large networks. arXiv [physics.soc-ph] (2008)) (igraph R package v1.2.5). To that end, a gene set network was constructed, where nodes were gene sets and edges are weighted based on shared gene membership by Jaccard index. Three cutoffs for the Jaccard index (0.9, 0.95, 0.99) were applied before clustering to produce different clustering resolutions. The clustering was repeated twice, considering membership by shared drivers or any shared genes between the gene sets. Results were reviewed and biological processes were generalized manually. Candidate genes that were not enriched in gene sets by this process were assigned to pathways by data curation (FIG. 7A).

### Immunoglobulin (IG) Gene Characterization

The IG heavy (IGH) and light (IGL) chain gene rearrangements and mutational status were obtained from WGS/WES and RNA-seq using IgCaller (v1.1) (Nadeu, F. et al. IgCaller for reconstructing immunoglobulin gene rearrangements and oncogenic translocations from whole-genome sequencing in lymphoid neoplasms. Nat. Commun. 11, 3390 (2020)) and MiXCR (v.3.0.10) (Bolotin, D. A. et al. MiXCR: software for comprehensive adaptive immunity profiling. Nat. Methods 12, 380-381 (2015)), respectively. The rearrangements obtained were visually inspected in IGV (Robinson, J. T. et al. Integrative genomics viewer. Nat. Biotechnol. 29, 24-26 (2011)). IGH gene rearrangements were complemented with Sanger sequencing available for 1085 cases. The IGHV (heavy chain variable region of immunoglobulin genes) mutational status obtained by IgCaller (WGS/WES) and MiXCR were concordant in 506/516 (98%) cases with an IGH rearrangement identified by both methods. The 10 discordant cases were classified based on the IGHV (heavy chain variable region of immunoglobulin genes) mutational status determined by Sanger sequencing (concordant with MiXCR in 8 cases and with IgCaller in 2). IgCaller/MiXCR and Sanger sequencing were concordant in 903/925 (98%) of the cases with an IGH gene rearrangement obtained by both methodologies. The result obtained by IgCaller/MiXCR was used in the 22 discordant cases after careful examination of the sequences. Note that in 12/22 cases the results obtained by IgCaller and MiXCR were concordant. For the remaining 10 cases, only IgCaller or MiXCR results were available. The IGHV (heavy chain variable region of immunoglobulin genes) mutational status of 14 cases carrying a mix of mutated and unmutated IGH gene rearrangements was considered as “not available”. Similarly, the IGH genes in 43 cases carrying two IGH rearrangements (the previous 14 cases with mixed IGHV (heavy chain variable region of immunoglobulin genes) mutational status and 29 cases with two mutated or two unmutated IGH gene rearrangements) were considered as “not available”. Altogether, 1136/1154 (98%) cases were classified based on their IGHV (heavy chain variable region of immunoglobulin genes) mutational status. To study B-cell receptor (BCR) stereotypy, the 19 major stereotype subsets were annotated using the ARResT/AssignSubsets online tool (Bystry, V. et al. ARResT/AssignSubsets: a novel application for robust subclassification of chronic lymphocytic leukemia based on B cell receptor IG stereotypy. Bioinformatics 31, 3844-3846 (2015)).

IGL gene rearrangements obtained by IgCaller and MiXCR were concordant in all but five cases with both methods available (581/586, 99%). The output of MiXCR was accepted in the five discordant cases after manual revision. As performed for IGH gene rearrangements, cases carrying two IG populations with distinct IG gene rearrangements were blacklisted from the IGL gene annotation. To properly characterize the IGLV3-21^{R110}, IGLV3-21 rearranged sequences reported by IgCaller were manually curated to phase single nucleotide polymorphisms with the rearranged allele, as previously described (Nadeu, F. et al. IGLV3-21R110 identifies an aggressive biological subtype of chronic lymphocytic leukemia with intermediate epigenetics. Blood (2020) doi:10.1182/blood.2020008311). Curated IGLV3-21-rearranged sequences from IgCaller and original IGLV3-21-rearranged sequences from MiXCR (in which the manual phasing of polymorphisms is not needed) were used as input of IMGT/V-QUEST (v3.5.18; release 202018-4) (Brochet, X., Lefranc, M.-P. & Giudicelli, V. IMGT/V-QUEST: the highly customized and integrated system for IG and TR standardized V-J and V-D-J sequence analysis. Nucleic Acids Res. 36, W503-8 (2008)) to annotate the IGLV3-21 allele, the motifs involved in BCR-BCR interactions [lysine (K) 16 and aspartates (D) 50 and 52], and the presence of the glycine to arginine mutation at position 110 (R110) (Nadeu, F. et al. IGLV3-21R110 identifies an aggressive biological subtype of chronic lymphocytic leukemia with intermediate epigenetics. Blood (2020) doi:10.1182/blood.2020008311). Overall, IGLV3-21^{R110} status was determined in 1128/1154 (97.7%) cases.

### RNA-Seq Analysis

RNA-seq data was processed in Terra using the GTEx V7 pipeline (github.com/broadinstitute/gtex-pipeline). Briefly, reads were aligned with STAR (v2.6.1d) (Dobin, A. et al. STAR: ultrafast universal RNA-seq aligner. Bioinformatics 29, 15-21 (2013)) to hg19 (b37) using the GENCODE v19 annotation, and quality control metrics and gene expression were computed with RNA-SeQC (v2.3.6) (Graubert, A., Aguet, F., Ravi, A., Ardlie, K. G. & Getz, G. RNA-SeQC 2: Efficient RNA-seq quality control and quantification for large cohorts. Bioinformatics (2021) doi:10.1093/bioinformatics/btab135). A collapsed version of the GENCODE annotation was used to quantify gene-level expression (available from gs://gtex-resources/GENCODE/gencode.v19.genes.v7.collapsed_only.patched_contigs.gtf). Transcripts per million (TPMs) were used for sample clustering, while gene counts were used for differential gene expression, as required.

### RNA Expression Cluster Detection

Gene-level transcripts per million (TPMs) were estimated with RNA-SeQC (v2.3.6) for RNA-seq from 610 treatment-naive CLL. Genes expressed at less than 0.1 transcripts per million (TPM) in 10% of samples were discarded, retaining 11,119 genes, which were batch corrected (as described below), followed by selection of the top 2,500 most varying genes. The clustering methodology combined consensus hierarchical clustering and Bayesian non-negative matrix factorization (BayesNMF), as previously described (Robertson, A. G. et al. Comprehensive Molecular Characterization of Muscle-Invasive Bladder Cancer. Cell 171, 540-556.e25 (2017)). Briefly, the method computed a distance matrix 1−C, where element Cᵢⱼ represented the Spearman correlation between samples i and j across the 2,500 genes. It used the distance matrix to perform iterations of standard hierarchical clustering with 80% sample resampling for 250 iterations per value of parameter K, where K represents the cutoff for the number of clusters running from 2 to 20. The result was the cumulative consensus matrix M, where Mᵢⱼ represents the number of times samples i and j shared cluster membership, which was then normalized by the total number of iterations to create the matrix M*. Next, BayesNMF was performed on M* to identify the optimal number of clusters K* and computed the strength of association of each sample to each cluster. The maximum association determined final cluster assignment. By parallelization, the number of independent BayesNMF runs was increased from 20 to 1000, 77.4% of which converged to the dominant result of K*=8 clusters (20% K*=7, 1.8% K*=6).

### RNA-Seq Batch Effect Correction

Preprocessing of RNA-seq data for expression cluster detection was undertaken to address batch effects between samples collected at different centers and processed by different protocols. To that end, a comprehensive set of covariates was assembled that allowed for adequate control for technical artifacts: (i) Quality metrics from RNA-SeQC v2.3.6 (Graubert, A., Aguet, F., Ravi, A., Ardlie, K. G. & Getz, G. RNA-SeQC 2: Efficient RNA-seq quality control and quantification for large cohorts. Bioinformatics (2021) doi:10.1093/bioinformatics/btab135); (ii) CIBERSORT (Chen, B., Khodadoust, M. S., Liu, C. L., Newman, A. M. & Alizadeh, A. A. Profiling Tumor Infiltrating Immune Cells with CIBERSORT. Methods Mol. Biol. 1711, 243-259 (2018)) relative immune cell composition estimates (cibersort.stanford.edu/) where B-cell estimates were excluded to prevent masking CLL-intrinsic signals; (iii) PEER factors (Stegle, O., Parts, L., Durbin, R. & Winn, J. A Bayesian framework to account for complex non-genetic factors in gene expression levels greatly increases power in eQTL studies. PLoS Comput. Biol. 6, e1000770 (2010)); (iv) Sex, which was systematically inferred by KMeans clustering (sklearn v0.21.3) using XIST and RPS4Y1 gene expression; (v) explicit sequencing batch if available; (vi) sequencing center (Broad Institute or Barcelona); (vii) a metric devised to estimate the sample processing artifact described in Dvinge et al (Dvinge, H. et al. Sample processing obscures cancer-specific alterations in leukemic transcriptomes. Proceedings of the National Academy of Sciences 111, 16802-16807 (2014)). This metric was computed by Spearman correlation between a sample's expression profile to the genes reported by Dvinge et al to be differentially expressed after 48 hours of incubation at suboptimal temperatures. However, to reduce the potential contribution of CLL-related expression to this metric, the correlation was computed by focusing on 3,682 differentially expressed genes that have been previously defined as house-keeping genes (Eisenberg, E. & Levanon, E. Y. Human housekeeping genes, revisited. Trends Genet. 29, 569-574 (2013)). Of note, covariates from RNA-SeQC (Graubert, A., Aguet, F., Ravi, A., Ardlie, K. G. & Getz, G. RNA-SeQC 2: Efficient RNA-seq quality control and quantification for large cohorts. Bioinformatics (2021) doi:10.1093/bioinformatics/btab135) and CIBERSORT were converted to PCA space. Top PCs and PEER factors were selected as appropriate. Batch correction for expression cluster (EC) detection was performed by including the covariates as fixed effects in a linear model to regress out effects they were associated with, and sample clustering was performed on the resulting residuals.

### Marker Gene Detection and Differential Expression Analysis

To identify marker genes per expression cluster (FIG. 3B), a second non-negative matrix factorization step was applied, as previously described (Robertson, A. G. et al. Comprehensive Molecular Characterization of Muscle-Invasive Bladder Cancer. Cell 171, 540-556.e25 (2017)). However, in this study, batch-corrected transcripts per million (TPMs) were used and a fold-change of 1.5 was required between each cluster and all others. Markers selected were limited to the top 10 most up and down regulated genes per expression cluster (EC) (Tables 3A-3B and 4). Additionally, limma-voom (Ritchie, M. E. et al. limma powers differential expression analyses for RNA-sequencing and microarray studies. Nucleic Acids Res. 43, e47 (2015); Law, C. W., Chen, Y., Shi, W. & Smyth, G. K. voom: Precision weights unlock linear model analysis tools for RNA-seq read counts. Genome Biol. 15, R29 (2014)) was used to identify differentially expressed genes between each expression cluster (EC) and all others (FIG. 14A). The same covariates used for RNA-seq batch effect correction for expression cluster discovery were included in the models, while using unmodified gene counts from RNA-SeQC (Graubert, A., Aguet, F., Ravi, A., Ardlie, K. G. & Getz, G. RNA-SeQC 2: Efficient RNA-seq quality control and quantification for large cohorts. Bioinformatics (2021) doi:10.1093/bioinformatics/btab135). Genes with q<0.05 and absolute fold-change greater than 1.5 were considered differentially expressed (Tables 3A-3B and 4).

### Gene Set Enrichment Analysis for Expression Clusters (ECs)

Gene set enrichment per each expression cluster was performed using fgsea (github.com/ctlab/fgsea) (Korotkevich, G. et al. Fast gene set enrichment analysis. bioRxiv 060012 (2021) doi:10.1101/060012), which was applied to the W matrix produced by the second BayesNMF step that detected marker genes associated with each expression cluster (EC) (see Robertson et al (Robertson, A. G. et al. Comprehensive Molecular Characterization of Muscle-Invasive Bladder Cancer. Cell 171, 540-556.e25 (2017)) for details). In essence, this represents gene lists ranked by their association with each EC, ranging from most positively associated to most negatively associated. Gene sets from MSigDB v7.0 were used, aggregating Hallmark, C5:GO:BP and C2:CP:REACTOME collections. Analysis was restricted to gene sets of size 12 to 500, and q<0.1 was required. For further confidence, we applied Gene Set Variation Analysis (GSVA) from the gsva R package (Hänzelmann, S., Castelo, R. & Guinney, J. GSVA: gene set variation analysis for microarray and RNA-seq data. BMC Bioinformatics 14, 7 (2013)) using the top 2500 most varying genes. GSVA estimates were summarized per expression cluster (EC) and mean differences computed between each expression cluster (EC) and all others. The intersection of results from fgsea and GSVA was retained.

Next, to identify related biological processes and remove redundancy in overlapping gene sets, significant gene sets were clustered using Louvain clustering (Blondel, V. D., Guillaume, J.-L., Lambiotte, R. & Lefebvre, E. Fast unfolding of communities in large networks. arXiv [physics.soc-ph] (2008)) (igraph R package v1.2.5). To that end, a gene set network was constructed, where nodes were gene sets and edges were weighted based on shared gene membership by Jaccard index (using genes in the “leading edge” reported by fgsea). Three cutoffs for Jaccard index (0.8, 0.9, 0.95) were applied before clustering to produce different clustering resolutions. Finally, results were reviewed and biological processes were generalized manually. Only gene sets with absolute NES scores >2 from fgsea and a >0.1 difference in mean GSVA score between the respective expression cluster (EC) and all other samples were considered.

### Detection of Statistically Significant Pairwise Associations of Molecular Features

To identify statistically significant pairwise associations of molecular features (e.g., association of expression clusters (ECs) with candidate drivers; FIG. 3B), the curveball permutation algorithm (Strona, G., Nappo, D., Boccacci, F., Fattorini, S. & San-Miguel-Ayanz, J. A fast and unbiased procedure to randomize ecological binary matrices with fixed row and column totals. Nat. Commun. 5, 4114 (2014)) was applied to a comprehensive sample annotation table to generate the null distribution of the p-value from one-sided Fisher's Exact tests for each pair of features. The sample annotation table contained binary indicators for all sSNV/indel drivers and somatic copy number alteration (sCNA) drivers identified, in addition to U1 mutation, IGLV3-21^{R110} mutation, IGHV (heavy chain variable region of immunoglobulin genes) mutational status, expression clusters (ECs) and epitypes. Samples that had DNA, RNA and methylation data were focused upon, and they were also required to be treatment-naive (n=506). The goal of the curveball algorithm was to estimate an accurate null distribution through controlling the sample-level driver mutation rates, which reduced false positive associations caused by background mutation burdens. 5000 curveball permutation iterations were applied to generate this null distribution and then the observed p-value was compared against it to get the empirical p-value for co-occurring and mutual-exclusive patterns for each feature pair. The Benjamini-Hochberg procedure was then applied to the empirical p-values and the significant events were selected (q<0.1) (Benjamini, Y. & Hochberg, Y. Controlling the false discovery rate: A practical and powerful approach to multiple testing. J. R. Stat. Soc. 57, 289-300 (1995)).

### Expression Cluster Machine-Learning Classifier

The 610 treatment-naive RNA-seqs of the expression cluster (EC) discovery set were split into a training set (n=487, 80%) and test set (n=123, 20%). The latter was used to assess performance after final model selection. Features used in the model were derived from differential expression results between expression clusters (ECs) using limma-voom (Ritchie, M. E. et al. limma powers differential expression analyses for RNA-sequencing and microarray studies. Nucleic Acids Res. 43, e47 (2015)) on training set samples. Models were trained using the RandomForestClassifier class in the sklearn (v0.22.2) Python package (with parameter class_weight=“balance_subsample” to mitigate class imbalance in the models). Hyperparameters were optimized using 5-fold cross validation and model performance was evaluated by the harmonic mean of overall accuracy and macroF1 (mean F1 across ECs). The final performance metric per hyper-parameter set was the mean of this value across cross-validation folds. Hyperparameters screened included forest size (500, 1000), number of most differentially expressed genes used from each comparison in limma-voom (5, 10, 20, 50) and oversampling method from the imblearn package (v0.6.2) used to improve performance (ADASYN, BorderlineSMOTE, SMOTE, SVMSMOTE or None). DESeq-normalized transcripts Per Million (TPMs) were used primarily and the process was repeated for batch-corrected transcripts Per Million (TPMs) to assess the impact of batch-correction on performance. Reported accuracy metrics were computed by applying the selected models to the test set.

### Stability Assessment of Expression Clusters

CLL RNA-seq data generated across multiple timepoints was analyzed prior to treatment from 19 patients (Gruber, M. et al. Growth dynamics in naturally progressing chronic lymphocytic leukaemia. Nature 570, 474-479 (2019)), focusing on two time points per patient in 18 of 19 cases. For one patient, CRC-0019, all 6 samples available were analyzed prior to treatment. The machine learning expression cluster (EC) classifier was applied to these 42 samples to obtain predicted expression cluster (EC) assignments. Importantly, to avoid biases for these patient samples, the classifier was retrained while excluding these patients from the training process. Then, to test if the assignment of expression clusters (ECs) was consistent over time more than expected by chance, a permutation test was performed, randomizing all labels among the 42 samples 1,000,000 times. For each permutation a value Hₚₑᵣₘ was computed by the sum of Shannon's entropy per patient. For example, a patient with consistent assignment in 2 samples contributed 0 bits to Hₚₑᵣₘ, whereas a patient with two different labels contributed 1 bit. The mean Hₚₑᵣₘ value was 10.47, compared to Hᵣₑₐₗ from the actual data that was 2.77. No randomizations were as low as this, providing a p-value <10⁻⁶ in support of expression cluster (EC) stability. This was based on stability in 15 of 19 patients, where 2/15 were classified differently than in the expression cluster (EC) discovery process. Considering 13/19 (68.4%), expression clusters (ECs) were consistent over time in most patients.

### DNA Methylation Data Processing

DNA methylome data was analyzed for a total of 1,037 samples, including 490 samples profiled with Illumina 450k array previously analyzed (Duran-Ferrer, M. et al. The proliferative history shapes the DNA methylome of B-cell tumors and predicts clinical outcome. Nature Cancer 1, 1066-1081 (2020)) (EGA accession EGAD00010001975), and 547 samples profiled using reduced representation bisulfite sequencing (RRBS, with either single-end (SE), or paired-end (PE) approaches) (Landau, D. A. et al. Locally disordered methylation forms the basis of intratumor methylome variation in chronic lymphocytic leukemia. Cancer Cell 26, 813-825 (2014)). A pipeline in Terra was developed to obtain the CpG methylation estimates from RRBS data. First, FASTQC (bioinformatics.babraham.ac.uk/projects/fastqc/) and MultiQC (Ewels, P., Magnusson, M., Lundin, S. & Käller, M. MultiQC: summarize analysis results for multiple tools and samples in a single report. Bioinformatics 32, 3047-3048 (2016)) were used for quality control. Trimming was applied to the PE samples as appropriate for the RRBS protocol. Next, reads were aligned to hg19 using BSMAP (Xi, Y. & Li, W. BSMAP: whole genome bisulfite sequence MAPping program. BMC Bioinformatics 10, 232 (2009)) (v2.90) and methylation was called with the mcall module from the MOABS package (Sun, D. et al. MOABS: model based analysis of bisulfite sequencing data. Genome Biol. 15, R38 (2014)) (v1.3.9.6). For SE samples, BSMAP was run with flags “-v 0.1 -s 12 -q 20 -w 100 -S 1 -u -R -D C-CGG -r 0”, and for PE samples with “-v 0.1 -s 12 -q 20 -w 100 -S 1 -u -R -r 0”. mcall was run with flag “-F 256”, for primary alignments only. For downstream analysis, only CpGs covered by at least 5 reads were retained. 14 samples were then removed from the initial 1,037, since they did not pass the filtering criteria due to poor bisulfite conversion rates, poor alignment metrics, suspected contaminations from other samples, extremely low number of methylated CpGs, and/or very low number of CpGs with 5 reads compared to the general distribution. After all filtering criteria, a total of 1,023 samples were used for all downstream analyses. From these 1,023 samples, 24 were profiled twice with different platforms and were used to validate the robustness of the new epiCMIT (Duran-Ferrer, M. et al. The proliferative history shapes the DNA methylome of B-cell tumors and predicts clinical outcome. Nature Cancer 1, 1066-1081 (2020)) epigenetic mitotic clock across platforms (18 profiled with Illumina 450k vs RRBS-PE, and 6 profiled with RRBS-PE vs RRBS-SE). In these 24 cases, the platform with more CpGs covered across all samples was prioritized (from the highest to lowest priority, Illumina 450k>RRBS-PE>RRBS-SE). The remaining 999 unique samples included 490 profiled by Illumina 450k array, 390 by RRBS-SE and 119 by RRBS-PE (3 samples were not included in consensus matrices due to lower number of CpGs, including 2 RRBS-SE and 1 RRBS-PE samples). The consensus matrices for each platform with shared CpGs across samples contained 447,800 CpGs and 490 samples for Illumina 450k data; 44,363 CpGs and 388 samples for RRBS-SE data; and 173,808 CpGs and 136 samples for RRBS-PE data [18 of these 136 samples were only used to test epiCMIT robustness across platforms, as they were already profiled with Illumina 450k; 6 of the remaining 118 RRBS-PE samples were also profiled with RRBS-SE to test epiCMIT robustness across platforms (analyzed separately and not included in the RRBS-SE consensus matrix), but were subsequently discarded and only their corresponding RRBS-PE samples were retained according to the aforementioned platform prioritization scheme]. These consensus matrices were used to perform principal component analyses (PCA) and in the case of RRBS data, also to assign CLL epitypes.

### CLL Epitype Classification

The CLL epitypes were calculated for all 1,023 450k/RRBS samples. In the case of Illumina 450k data, a recently published algorithm was used which uses 4 CpGs and is suitable for both Illumina 450k and EPIC arrays (Duran-Ferrer, M. et al. The proliferative history shapes the DNA methylome of B-cell tumors and predicts clinical outcome. Nature Cancer 1, 1066-1081 (2020)). For RRBS data, the previously created consensus matrices created for RRBS-SE and RRBS-PE platforms were used separately and the following strategy was used: CLL patients with 100% and ≤95% IGHV (heavy chain variable region of immunoglobulin genes) identities were selected to perform differential DNA methylation analysis with mean methylation fraction differences between groups of at least 0.5. These IGHV (heavy chain variable region of immunoglobulin genes) cutoffs yielded 168 and 80 samples for RRBS-SE data, and 67 and 13 samples for RRBS-PE data with IGHV (heavy chain variable region of immunoglobulin genes) identities of 100% and ≤95%, respectively. These stringent cutoffs were imposed for both IGHV (heavy chain variable region of immunoglobulin genes) and DNA methylation differences to avoid borderline cases, compared with the traditional 98% IGHV (heavy chain variable region of immunoglobulin genes) and 0.25 methylation difference cutoffs. This filtering criteria translated into clearer signatures consisting of 32 and 153 differentially methylated CpGs for RRBS-SE and RRBS-PE data, respectively (FIG. 12D). These CpGs were then used to perform consensus clustering with ConsensusClusterPlus R package v.1.52.0 (Wilkerson, M. D. & Hayes, D. N. ConsensusClusterPlus: a class discovery tool with confidence assessments and item tracking. Bioinformatics 26, 1572-1573 (2010)) with 10,000 permutations allowing from K=2 to K=7 groups, which robustly identified 3 consensus groups in both RRBS data types (FIGS. 12A-12C). Each sample was assigned a probability to belong to each of the groups (using the calcICL function). Samples where the maximum probability was below 0.5 or where 2 epitypes had a probability above 0.35 were considered as unclassified cases. In the 3 samples (2 RRBS-SE and 1 RRBS-PE) not included in the consensus matrices, the same strategy was used to find the CLL epitypes using the intersection of CpGs from both matrices used for consensus clustering (i.e., the 32-CpG and 153-CpG matrices for RRBS-SE and RRBS-PE data). In these cases, the epitype predictions were additionally verified using PCAs with all the shared CpGs with the rest of the samples, which further supported the assigned epitype.

Development of the epiCMIT Mitotic Clock for Next Generation Sequencing Data

The epigenetic mitotic clock, epiCMIT, was originally created with Illumina array data and thus is suitable for both 450k and EPIC arrays (Duran-Ferrer, M. et al. The proliferative history shapes the DNA methylome of B-cell tumors and predicts clinical outcome. Nature Cancer 1, 1066-1081 (2020)). The coverage of the original epiCMIT-CpGs based on Illumina 450k data in more targeted sequencing approaches like RRBS can be greatly compromised depending on the sequencing depth of samples or the enrichment towards particular regions of the genome. To overcome this, the epiCMIT-CpGs catalogue was expanded using high coverage whole genome bisulfite sequencing (WGBS) data from a previous publications including 15 samples covering the entire B-cell maturation spectrum (Kulis, M. et al. Whole-genome fingerprint of the DNA methylome during human B cell differentiation. Nat. Genet. 47, 746-756 (2015); Kretzmer, H. et al. DNA methylome analysis in Burkitt and follicular lymphomas identifies differentially methylated regions linked to somatic mutation and transcriptional control. Nat. Genet. 47, 1316-1325 (2015); Kulis, M. et al. Epigenomic analysis detects widespread gene-body DNA hypomethylation in chronic lymphocytic leukemia. Nat. Genet. 44, 1236-1242 (2012)) (FIG. 12E). Briefly, the genome was segmented into 12 CHMM states with 200 bp resolution using the CHMM software (Ernst, J. & Kellis, M. ChromHMM: automating chromatin-state discovery and characterization. Nat. Methods 9, 215-216 (2012)) fed with 6 histone marks including H3K27ac, H3K4me1, H3K4me3, H3K36me3, H3K9me3 and H3K27me3 available for 15 normal and 16 neoplastic B cell samples. Normals included 6 naive B cells, 3 germinal-center B cells, 2 memory B cells and 3 tonsillar plasma cell samples. Neoplasia samples included 5 mantle cell lymphoma, 7 CLL and 4 multiple myeloma samples. These 12 chromatin states were ActProm (active promoter, with H3K27ac and H3K4me3), WkProm (weak promoter, with H3K4me1 and H3K4me3), PoisProm (poised promoter, with H3K27me3, H3K4me1 and H3K4me3), StrEnh1 (strong enhancer 1, with H3K27ac, H3K4me1 and H3K4me3), StrEnh2 (strong enhancer 2, with H3K27ac and H3K4me1), WkEnh (weak enhancer, with H3K4me1), TxnTrans (transcription transition, with H3K36me3, H3K27ac and H3K4me1), TxnElong (transcription elongation, with H3K36me3), WkTxn (weak transcription, with low H3K36me3), H3K9me3 (H3K9me3-marked repressed heterochromatin), H3K27me3 (H3K27me3-marked repressed heterochromatin) and Het;LowSign (low-signal heterochromatin, with the absence of all six histone marks). Next, we selected CpGs located in repressive regions, including PoisProms, H3K27me3-repressed, H3K9me3 regions and Het;LowSign heterochromatin states. Afterwards, only CpGs showing extensive methylation differences (>0.5 difference in methylation fraction) between the lowly divided hematopoietic stem cell (HPC) and the highly divided bone-marrow plasma cells (bmPC) were retained, yielding 4,169 epiCMIT-hyper-CpGs (gaining methylation in H3K27me3 and PoisProm regions) and 808,872 epiCMIT-hypo-CpGs (CpGs losing methylation in H3K9me3 and Het;LowSign) in the hg38 genome assembly. Finally, the epiCMIT-hyper and epiCMIT-hypo scores were calculated as previously described (Duran-Ferrer, et al.) and the higher value in each sample was selected separately, which is different than the original strategy for Illumina array data where all samples shared the same epiCMIT-CpGs for the calculations (Duran-Ferrer, et al.) (only CpGs covered by at least 5 reads were used). This strategy was implemented to maximize the number of epiCMIT-CpGs in each sample, as only 124 and 311 epiCMIT-CpGs of the extended epiCMIT-CpGs catalogue were present in RRBS-SE and RRBS-PE consensus matrices, respectively. The new approach was validated using 24 samples profiled twice with different platforms, including 18 samples profiled with Illumina 450k and RRBS-PE, and 6 samples with RRBS-PE and RRBS-SE (FIG. 12F). In the samples profiled with Illumina 450k, the original epiCMIT-CpGs were used, whereas in RRBS data the available epiCMIT-CpGs was used in each sample of the extended catalogue of epiCMIT-CpGs based on WGBS data. These analyses showed that (i) the new epiCMIT approach was highly correlated with the original one, (ii) the epiCMIT could be calculated with varying numbers of epiCMIT-CpGs (with a minimum of around 800 epiCMIT-CpGs), and (iii) epiCMIT could be calculated with minimal impact due to different batches and platforms used. These statements were further supported by the PCA analyses with Illumina 450k data (ICGC cohort) and RRBS-SE data (DFCI and GCLLSG cohorts, n=93 and n=295, respectively) (FIG. 3A) and RRBS-PE (data not shown), in which the epiCMIT gradient was similar in both platforms and unaffected by different cohorts.

### H3K27Ac ChIP-Seq Analysis of Expression Clusters

To study the regulatory landscape of each ECs, previously analyzed cases with H3K27ac ChIP-seq were used (n=104), from which 70 cases had available RNA-seq and DNA methylation data. In these 70 samples, the number of cases for each expression cluster (EC) was: EC-m1=11, EC-u1=24, EC-m2=5, EC-o=2, EC-u2=5, EC-m3=10, EC-m4=12 and EC-i=1. From the 70 cases with available expression cluster (EC) classification, those expression clusters (ECs) with at least 5 cases (EC-o and EC-i were excluded) were selected and a differential analysis was performed using DESeq2 (Love, M. I., Huber, W. & Anders, S. Moderated estimation of fold change and dispersion for RNA-seq data with DESeq2. Genome Biol. 15, 550 (2014)) with raw H3K27ac counts. Genome-wide analyses was performed comparing each expression cluster (EC) versus the others using a consensus matrix with 100,640 regions showing at least one H3K27ac peak in one of the 104 samples, and those regions with an FDR≤0.05 in any of the comparisons were retained. This data was used in FIG. 14A.

Additionally, differential analyses was performed focused on those regulatory regions associated with the marker genes of each expression cluster (EC) (FIG. 3G). To do so, all expression cluster (EC) marker gene coordinates were selected and extended 2,000 bp upstream of their corresponding transcription start sites. These regions were then intersected with the consensus matrix (n=100,640) and a differential DESeq2 (Love, M. I., Huber, W. & Anders, S. Moderated estimation of fold change and dispersion for RNA-seq data with DESeq2. Genome Biol. 15, 550 (2014)) analysis was performed with each expression cluster (EC) versus all the others and identified regions with FDR≤0.05 (FIGS. 13I-13J). These results were used for the H3K27ac annotation of the marker genes in FIG. 3B.

### Statistical Methods

Unless otherwise stated, two-sided t-test was used for mean comparison and multiple testing was corrected to compute false discovery rate (FDR, q) by the Benjamini-Hochberg procedure (Benjamini, Y. & Hochberg, Y. Controlling the false discovery rate: A practical and powerful approach to multiple testing. J. R. Stat. Soc. 57, 289-300 (1995)). Categorical enrichments were computed using a two-sided Fisher's Exact test unless otherwise stated.

### Clinical Outcome Modeling

Failure-free survival (FFS) was calculated for treatment-naïve patients as the time from the date of the sequenced sample to the date of first treatment (“natural progression”), progression (if the patient was sampled at the time of enrollment on a clinical trial) or death, and censored at the last known event-free date. In the genetics-focused analysis (Tables 1A-1E and 2A-2E), the first event was defined as time to next treatment in patients who received therapy within 30 days. Subset analysis included patients who were treatment-naïve at the time of the sequenced sample and not enrolled on a therapeutic clinical trial; in this analysis, time between sample and date of first treatment was used. Overall survival (OS) was calculated as the time from the date of the sequenced sample to the date of death and censored at the date last known alive. Univariate and multivariable Cox regression models were constructed for each subset of data. Final models were selected using the glmnet function for regularized Cox regression using an elastic net penalty within the Coxnet package in R. Ten-fold cross-validation using the cv.glmnet function with a partial-likelihood deviance metric to minimize λ was performed and the minimum CV-error model was used. The alpha was set to 1 corresponding to a Lasso penalty. The maximum iterations (maxit) parameter was set to 1000. Features identified as having non-zero coefficient values using elastic net and selected in the final model were then included in a Cox regression model to obtain the hazard ratios. These hazard ratios estimated the magnitude of effect but p-values and confidence intervals are not readily interpretable in the elastic net model and are therefore not reported. For the integrated analysis of all available datatypes (Tables 5A-5D and 6A-6C), variables including expression cluster and epitype categories were dummy coded. Prognostic significance of expression cluster and IGHV (heavy chain variable region of immunoglobulin genes) status were also considered using a chi-squared test with the difference in −2 log likelihood (−2 log L) between models including somatic single nucleotide variants (sSNVs) and somatic copy number alterations (sCNAs). The Breslow approximation was used for handling ties in survival time.

### Non-Coding Driver Discovery Procedure

MutSig2CV-NC (Rheinbay, E. et al. Analyses of non-coding somatic drivers in 2,658 cancer whole genomes. Nature 578, 102-111 (2020)) (github.com/broadinstitute/getzlab-PCAWG-MutSig2CV_NC.git) was first used to identify candidate non-coding drivers in different genomic regions including enhancers, 3′ UTRs, 5′ UTRs, promoters and lncRNA genes. Then the stringent post-filtering steps described in detail in the Pan-cancer Analysis of Whole Genomes (PCAWG) Project's non-coding drivers paper (Bailey, et al) was followed on the candidate targets (q<0.5). In summary, the post-filters required:
1). at least three mutations are present in the candidate driver;
2). at least three patients have mutations in the candidate driver;
3). less than 50% of mutations are in palindromic DNA;
4). more than 50% of mutations are in mappable regions;
5). less than 35% of mutations have Activation-induced cytidine deaminase (AID)-related signatures attribution greater than 50%;
6). mutations pass manual review in IGV.

For candidate targets failing any of the above filters, their p-values were re-assigned to be 1. Finally, Benjamini-Hochberg multiple hypothesis correction was applied on the corrected p-values to get the post-filtered q-values. This provided 1 candidate (q<0.1): WDR74 which was reported in the aforementioned PCAWG paper (Rheinbay, et al). Additionally, RNA-seq analysis of mutated versus unmutated samples did not reveal a notable effect on gene expression of mutations in an extended list of candidate genes. Thus, novel non-coding drivers were not reported.

### Mutational Signatures Review

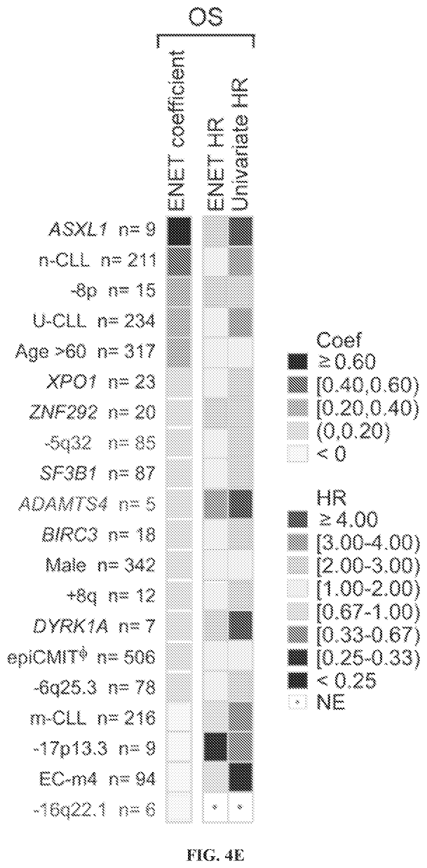

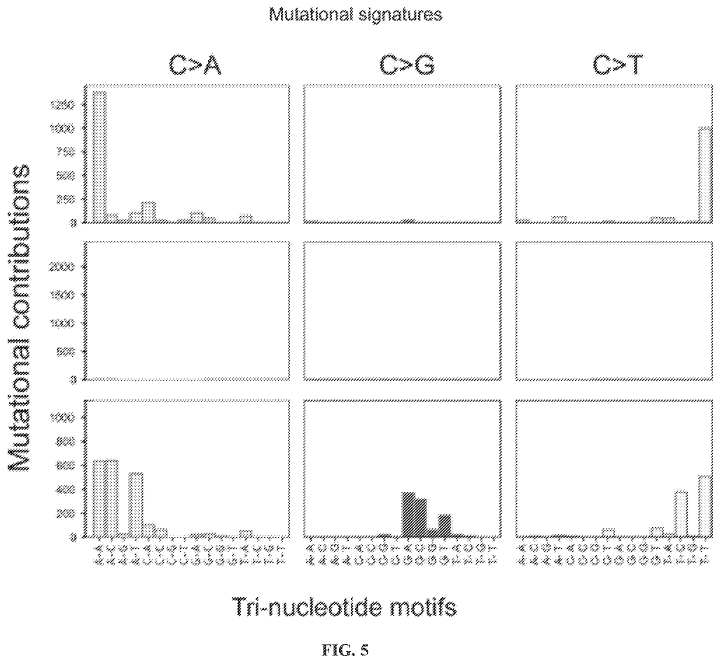

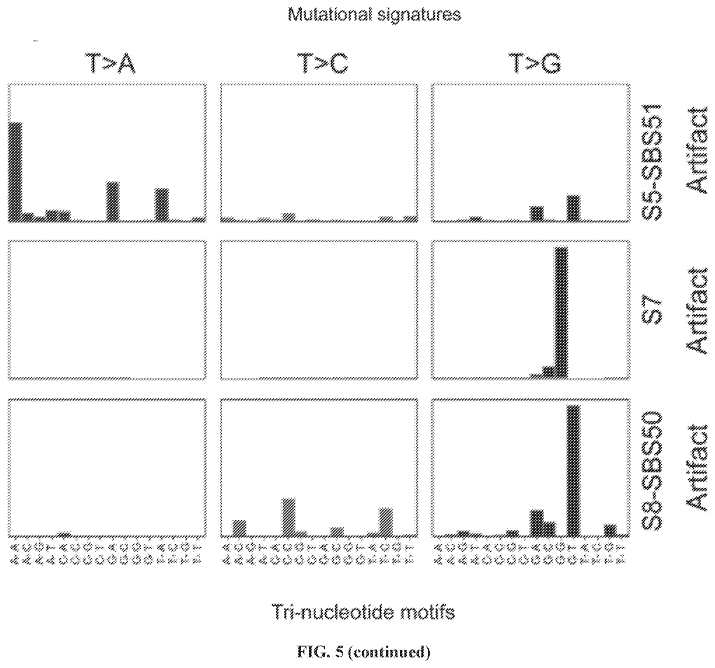

By applying SignatureAnalyzer (Kim, J. et al. Somatic ERCC2 mutations are associated with a distinct genomic signature in urothelial tumors. Nat. Genet. 48, 600-606 (2016)) to 177 WGS, 8 mutational signatures were observed acting in these samples. A careful review suggested that three signatures (S5, S7, S8; FIG. 5) might correspond to possible sequencing artifacts, and thus were removed from the main signatures plot in FIG. 11 depicting the 5 biological mutational processes identified by SignatureAnalyzer. Specifically, the cosine similarity between S5 and SBS51 (per COSMIC v3.1) is 0.82, while the cosine similarity between S8 and SBS50 (per COSMIC v3.1) is 0.74. S7 only contains one striking peak at G(T>G)G motif and thus it is assumed to be a bleed-through artifact.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A panel for characterizing chronic lymphocytic leukemia in a biological sample of a subject, the panel comprising two or more polypeptide markers selected from the following sets of polypeptide markers:
A) ABCA9, ACAP3, ACSM3, ADAP2, AF127936.7, ARHGAP33, ARMC7, ARRDC5, ARSD, ARSI, ASB2, ATP1A3, ATP2B1, ATPIF1, BASP1, BCL2A1, BCL7A, BCS1L, CAMK2A, CLDN23, CMTM7, COBLL1, CRELD2, CRY1, CTAGE9, CTLA4, DDR1, DKFZP761J1410, DPF3, EML6, ERRFI1, ESPNL, EZH2, FAHD2B, FAM109A, FBXO27, FGL2, FLJ20373, FMOD, GADD45A, GNAO1, GPR160, GPR34, GUCD1, HCK, HDAC4, HIP1R, HMCES, IGSF3, IQSEC1, ITGAX, KCNH3, KCNN3, KCTD3, KDM1B, KLK1, KSR1, LCN10, LINC00865, LPL, LRRK2, LUZP1, MAP4K4, MAPK4, MAST4, MPRIP, MRO, MSI2, MVB12B, MYBL1, MYC, MYL5, MYL9, MYO3A, NEDD9, NFKBIZ, NR2F6, NRIP1, NRSN2, NUGGC, P2RX1, PELI3, PIGB, PIP5K1B, PITPNC1, PLD1, PTPN7, QDPR, REPS2, RHBDF2, RIMKLB, RP11-134N1.2, RP11-265P11.1, RP11-453F18_B.1, RP11-456H18.2, RP1-90J20.12, SAMSN1, SCPEP1, SH3D21, SLC44A1, SLC4A7, SLC4A8, SMIM10, SPN, SSBP3, STAM, STX5, SYNGR3, TAS1R3, TBC1D2B, TBC1D9, TFEC, TIMELESS, TNFRSF13B, TNR, TOX2, TRIM7, TUBG2, VSIG10, WNT5A, ZMYND8, and ZNF804A,
B) ACAP3, ACSM3, AEBP1, AKT3, ARHGAP33, ARHGAP42, ARMC7, ARRDC5, ATPIF1, BACH2, BASP1, BCL7A, C17orf100, CBLB, CD72, CD86, CEACAM1, CHPT1, CLDN7, CMTM7, CNTNAP1, COBLL1, COL18A1, CRY1, CTLA4, EGR3, EML6, EZH2, FADS3, FCER1G, FCRL2, FGL2, FLJ20373, FMOD, GADD45A, GLIPR1, GNB4, GPR160, GPR34, GRIK3, GUCD1, HCK, HIP1R, HIVEP3, HMCES, IGF2BP3, IGSF3, IL21R, INPP5F, IQGAP2, IQSEC1, ITGAX, ITGB5, JDP2, KANK2, KCNH2, KDM1B, KLF3, LATS2, LCN10, LEF1, LPL, LRRK2, LUZP1, MAP4K4, MID1IP1, MMP14, MPRIP, MSI2, MYBL1, MYL9, MYLIP, MZB1, NBPF3, NRIP1, NRSN2, NUGGC, NXPH4, P2RX1, P2RX5, P2RY14, PDGFD, PIP5K1B, PITPNC1, PON2, PRICKLE1, PTPN7, RCN3, RDX, RHBDF2, RIMKLB, RNF135, RP11-145M9.4, RP11-268J15.5, RP11-463012.3, RP5-1028K7.2, SAMSN1, SCCPDH, SCD, SCPEP1, SDC3, SECTM1, SESN3, SH3BP2, SH3D21, SLC16A5, SLC19A1, SLC4A7, SPN, SSBP3, STX5, SUSD1, TBC1D2B, TBC1D9, TBKBP1, TCF7, TFEC, TGFBR3, TIGIT, TIMELESS, TMEM133, TNFRSF13B, TOX2, TRAK2, TTC39C, TUBG2, VPS37B, VSIG10, WNT9A, ZAP70, ZNF667-AS1, ZNF804A, and ZSWIM6,
(C) an Ec-i set comprising or consisting of polypeptide markers GRIK3, IQGAP2, FCER1G, STK32B, GADD45A, ITGAX, KLF3, RFTN1, PTK2, DFNB31, and ZMAT1;
(D) an EC-m1 set comprising or consisting of polypeptide markers TFEC, COL18A1, SLC19A1, NRIP1, KCNH2, P2RX1, ARRDC5, BEX4, and APP;
(E) an Ec-m2 set comprising or consisting of polypeptide markers EML6, HCK, CD1C, VPS37B, CYBB, NXPH4, BTNL9, KLRK1, IQSEC1, BANK1, LEF1, SH3D21, FMOD, SEMA4A, CTLA4, ADTRP, IGSF3, IGFBP4, PDGFD, and APOD;
(F) an Ec-m3 set comprising or consisting of polypeptide markers MS4A4E, MYL9, NT5E, MS4A6A, PITPNC1, CNTNAP2, IGF2BP3, WNT3, CLDN7, TCF7, BASP1, F1120373, MAP4K4, LRRK2, SAMSN1, CEACAM1, TNFRSF13B, PHF16, MID1IP1, and ABCA9;
(G) an Ec-m4 set comprising or consisting of polypeptide markers MYBL1, NUGGC, GNG8, AEBP1, HIP1R, LATS2, RIMKLB, EML6, FADS3, MBOAT1, LCN10, DCLK2, and GLUL;
(H) an Ec-o set comprising or consisting of polypeptide markers ACSM3, TOX2, PHF16, SESN3, TBC1D9, PIP5K1B, SIK1, DUSP5, GNG7, HIVEP3, MARCKSL1, GPR183, HRK, and PITPNC1;
(I) an Ec-u1 set comprising or consisting of polypeptide markers SEPT10, LDOC1, LPL, KANK2, SOWAHC, DUSP26, OSBPL5, WNT9A, FGFR1, GTSF1L, ADD3, AKT3, COBLL1, MNDA, FCRL3, FAM49A, FCRL2, SLC2A3, and MARCKS; and
(J) an Ec-u2 set comprising or consisting of polypeptide markers ITGB5, BCL7A, PPP1R9A, TSPAN13, SLC12A7, SSBP3, VASH1, SPG20, IL13RA1, NR3C2, TUBG2, ZNF804A, and IL2RA; or
fragments thereof, or sets of polynucleotides encoding such polypeptides or fragments thereof.

2-3. (canceled)

4. The panel of claim 1, wherein the markers are bound to a capture molecule.

5. The panel of claim 4, wherein the capture molecule is bound to a substrate.

6. A panel of capture molecules, wherein each capture molecule binds a marker of claim 1.

7. The panel of claim 6, wherein the capture molecules comprise an antibody or antigen binding fragment thereof.

8. The panel of claim 6, wherein the capture molecules comprise a polynucleotide.

9. A method of characterizing a chronic lymphocytic leukemia (CLL), the method comprising:
(A) measuring the level of each of a set of markers in a biological sample, wherein the set of biomarkers comprises two or more of markers selected from the sets of markers listed in claim 1, and
(B) using the measured levels to classify the CLL as having an expression subtype selected from Ec-i, EC-m1, EC-m2, EC-m3, EC-m4, EC-o, EC-u1, or EC-u2, thereby characterizing the CLL.

10-11. (canceled)

12. The method of claim 9, wherein (B) further comprises using the level of each biomarker as an input to a classifier to determine the expression subtype.

13. The method of claim 12, wherein the classifier is a machine learning classifier.

14-19. (canceled)

20. The method of claim 9, wherein the levels are measured using polynucleotide sequencing; RNA-seq, targeted sequencing, immunoassay or affinity capture, using a protein or nucleic acid biochip, mass spectroscopy, a capture molecule, or a NanoString assay.

21-27. (canceled)

28. The method of claim 27, wherein the capture molecule comprises a molecular identifier.

29. (canceled)

30. The method of claim 28, wherein the method comprises detecting the molecular identifier using FACS.

31. (canceled)

32. The method of claim 9, wherein measuring the levels is carried out on a plate, chip, beads, microfluidic platform, membrane, planar microarray, or suspension array.

33. A kit for characterizing a chronic lymphocytic leukemia (CLL), the kit comprising a set of capture molecules each of which specifically binds biomarkers of the panel of claim 1.

34. A method for selecting a subject having chronic lymphocytic leukemia (CLL) for inclusion in or exclusion from a clinical trial, the method comprising:
(A) characterizing the CLL according to the method of claim 9 to determine the expression subtype of the CLL,
(B) selecting the subject for inclusion in the clinical trial if the CLL has an expression subtype associated with sensitivity to a drug used in the clinical trial, and excluding the subject from the clinical trial if the CLL has an expression subtype associated with resistance to a drug used in the clinical trial.

35. A method for treating a selected subject having chronic lymphocytic leukemia (CLL), the method comprising:
administering an agent to a selected subject, wherein the subject is selected for treatment by characterizing marker expression in a biological sample of the subject using a panel of claim 1.

36. The method of claim 34, wherein the agent is a kinase inhibitor or a B-cell receptor pathway inhibitor.

37-39. (canceled)

40. The method of claim 34, wherein the agent is selected from the group consisting of 1-Ter-Butyl-3-P-Tolyl-1h-Pyrazolo[3,4-D]Pyrimidin-4-Ylamine, 4-HYDROXY-N′-(4-ISOPROPYLBENZYL)BENZOHYDRAZIDE, actinomycin D, afatinib, Amsacrine, and/or Vernakalant, Astemizole, AT13387, AZD7762, Azimilide, BAY 11-7085, Bepridil, Betrixaban, Bosutinib, BX912, Carvedilol, CCT241533, cephaeline, chaetoglobosin A, Chlorobutanol, Chlorpromazine, Ciprofloxacin, Cisapride, Clarithromycin, Cytarabine, dasatinib, Disopyramide, Dofetilide, Doxepin, Dronedarone, duvelisib, Erythromycin, everolimus, Flecainide, fludarabine, Fluoxetine, Fluvoxamine, Fostamatinib, Halofantrine, Hydroxyzine, ibrutinib, Ibutilide, idelalisib, Imipramine, Isavuconazole, Ketoconazole, KU-60019, KX2-391, Levomefolic acid, Loratadine, Methotrexate, MIS-43, MK-1775, MK-2206, navitoclax, Nefazodone, Nitazoxanide, NU7441, Pentoxifylline, Pentoxyverine, Perhexiline, PF 477736, Phenytoin, Phosphonotyrosine, Pimozide, Pitolisant, Potassium nitrate, Pralatrexate, Prazosin, Procainamide, Propafenone, PRT062607 HCl, Quercetin, Quinidine, rotenone, saracatinib, SD07, See comments, selumetinib, Semaglutide, Sertindole, SGI-1776, SNS-032, Sotalol, spebrutinib, TAE684, tamatinib, Tamoxifen, Tecastemizole, Terazosin, Terfenadine, thapsigargin, Thioridazine, Topiramate, Trimetrexate, venetoclax, Verapamil, vorinostat, and YM155.

41. (canceled)

42. The method of claim 34, wherein the agent used in the clinical trial is fludarabine, and wherein if the lymphocytic leukemia (CLL) has the expression subtype EC-m3, the subject is selected for inclusion in the clinical trial;
wherein the drug used in the clinical trial targets the B cell receptor pathway or PI3K/AKT, and wherein if the lymphocytic leukemia (CLL) has the expression subtype EC-m3, the subject is excluded from the clinical trial;
wherein the drug used in the clinical trial is ibrutinib or idelalisib, and wherein if the lymphocytic leukemia (CLL) has the expression subtype EC-m3, the subject is excluded from the clinical trial;
wherein the drug used in the clinical trial targets CDK2/7/9, and wherein if the lymphocytic leukemia (CLL) has the expression subtype EC-m4, the subject selected for inclusion in the clinical trial;
wherein the drug used in the clinical trial is SNS-032, and wherein if the lymphocytic leukemia (CLL) has the expression subtype EC-m4, the subject selected for inclusion in the clinical trial;
wherein the drug used in the clinical trial targets the B cell receptor pathway or BTK, and wherein if the lymphocytic leukemia (CLL) has the expression subtype EC-m4, the subject is excluded from the clinical trial;
wherein the drug used in the clinical trial is ibrutinib, and wherein if the lymphocytic leukemia (CLL) has the expression subtype EC-m4, the subject is excluded from the clinical trial;
wherein the drug used in the clinical trial targets apoptosis, BH3, and/or survivin, and wherein if the lymphocytic leukemia (CLL) has the expression subtype EC-u1, the subject is excluded from the clinical trial;
wherein the drug used in the clinical trial is venetoclax or navitoclax, and wherein if the lymphocytic leukemia (CLL) has the expression subtype EC-u1, the subject is excluded from the clinical trial;
wherein the drug used in the clinical trial targets DNA damage response, the B-cell receptor pathway, MAPK, PI3K/AKT, HSP90, or BCR/ABL, and wherein if the lymphocytic leukemia (CLL) has the expression subtype EC-u2, the subject is selected for inclusion in the clinical trial; or
wherein the drug used in the clinical trial is AZD7762, dasatinib, AT13387, ibrutinib, duvelisib, idelalisib, selumetinib, or PRT062607 HCl, and wherein if the lymphocytic leukemia (CLL) has the expression subtype EC-u2, the subject is selected for inclusion in the clinical trial.

43-52. (canceled)

53. The method of claim 35, wherein the subject is selected for administration of fludarabine if the expression subtype is EC-m3;
wherein the subject is selected for administration of a drug targeting CDK2/7/9 if the expression subtype is EC-m4;
wherein the subject is selected for administration of SNS-032 if the expression subtype is EC-m4;
wherein the subject is selected for administration of a drug targeting DNA damage response, the B-cell receptor pathway, MAPK, PI3K/AKT, HSP90, or BCR/ABL if the expression subtype is EC-u2;
wherein the subject is selected for administration of AZD7762, dasatinib, AT13387, ibrutinib, duvelisib, idelalisib, selumetinib, or PRT062607 HCl if the expression subtype is EC-u2;
wherein, if the CLL has an expression subtype associated with NRIP1, the subject is selected for administration of 4-HYDROXY-N′-(4-ISOPROPYLBENZYL)BENZOHYDRAZIDE;
wherein, if the CLL has an expression subtype associated with SLC19A1, the subject is selected for administration of an agent selected from the group consisting of Pralatrexate, Methotrexate, Levomefolic acid, Nitazoxanide, and Trimetrexate;
wherein, if the CLL has an expression subtype associated with KCNH2, the subject is selected for administration of an agent selected from the group consisting of Amsacrine, Astemizole, Azimilide, Bepridil, Betrixaban, Carvedilol, Chlorobutanol, Chlorpromazine, Ciprofloxacin, Cisapride, Clarithromycin, Disopyramide, Dofetilide, Doxepin, Dronedarone, Erythromycin, Flecainide, Fluoxetine, Fluvoxamine, Halofantrine, Hydroxyzine, Ibutilide, Imipramine, Isavuconazole, Ketoconazole, Loratadine, Nefazodone, Pentoxyverine, Perhexiline, Phenytoin, Pimozide, Pitolisant, Potassium nitrate, Prazosin, Procainamide, Propafenone, Quinidine, Sertindole, Sotalol, Tamoxifen, Tecastemizole, Terazosin, Terfenadine, Thioridazine, Verapamil, and Vernakalant;
wherein, if the CLL has an expression subtype associated with LPL, the subject is selected for administration of Semaglutide;
wherein, if the CLL has an expression subtype associated with HCK, the subject is selected for administration of an agent selected from the group consisting of 1-Ter-Butyl-3-P-Tolyl-1h-Pyrazolo[3,4-D]Pyrimidin-4-Ylamine, Phosphonotyrosine, Quercetin, Bosutinib, and Fostamatinib;
wherein, if the CLL has an expression subtype associated with NT5E, the subject is selected for administration of an agent selected from the group consisting of Pentoxifylline, and Cytarabine;
wherein if the CLL has an expression subtype associated with GRIK3, the subject is selected for administration of Topiramate.

54-64. (canceled)
